(19) United States
(12) Patent Application Publication
(10) Pub. No.: US 2024/0200154 A1
(43) Pub. Date: Jun. 20, 2024

(54) DETECTION OF SARS-COV-2 VARIANT

(71) Applicant: THE BOARD OF REGENTS OF THE UNIVERSITY OF TEXAS SYSTEM, Austin, TX (US)

(72) Inventor: Jeffrey A. SORELLE, Dallas, TX (US)

(73) Assignee: THE BOARD OF REGENTS OF THE UNIVERSITY OF TEXAS SYSTEM, Austin, TX (US)

(21) Appl. No.: 18/286,855

(22) PCT Filed: Apr. 14, 2022
(86) PCT No.: PCT/US2022/024930

§ 371 Date: Oct. 13, 2023

Related U.S. Application Data
(60) Provisional application No. 63/174,787, filed on Apr. 14, 2021.
(60) Provisional application No. 63/299,618, filed on Jan. 14, 2022.

(51) Int. Cl. C12Q 1/70; C12Q 1/6844; C12Q 1/6876

(52) U.S. Cl. CPC C12Q 1/701; C12Q 1/6844; C12Q 1/6876; C12Q 2600/156; C12Q 2600/16

(57) ABSTRACT

Provided herein are compositions and methods for detecting novel SARS-COV-2 variants. Disclosed herein are nucleic acid constructs that can be used as primers in various molecular assays to identify and distinguish new variants of the SARS-COV-2 virus. Also provided are methods of using said nucleic acid constructs to identify and distinguish new variants of the SARS-COV-2 virus.

### CROSS REFERENCE TO RELATED APPLICATION

This application claims the benefit of U.S. Provisional Application No. 63/174,787 filed on Apr. 14, 2021 and U.S. Provisional Application No. 63/299,618 filed Jan. 14, 2022, the disclosures of which are hereby incorporated by reference in their entirety.

### CROSS REFERENCE TO RELATED APPLICATION INCORPORATION BY REFERENCE OF SEQUENCE LISTING PROVIDED ELECTRONICALLY

This application contains a Sequence Listing that has been submitted in ASCII format via EFS-Web and is hereby incorporated by reference in its entirety. The ASCII copy is named “106546-715025_UTSD_3878_SequenceListing_ST25.txt”, was created on Apr. 14, 2022, and is 63,000 bytes in size.

### BACKGROUND

### 1. Field

present disclosure is generally directed to compositions and methods for detecting novel SARS-COV-2 variants. Disclosed herein are nucleic acid constructs that can be used as primers in various molecular assays to distinguish new variants of the SARS-COV-2 virus. Also provided are methods of use thereof.

### 2. Discussion of Related Art

SARS-COV-2, which causes COVID-19, is a coronavirus that has led to a global shutdown and millions of deaths. These variants impact transmission, vaccine response, mortality, and drug resistance (monoclonal antibodies). Therefore, methods for real-time identification of SARS-CoV-2 variant strains are of exigent need in both clinical and public health arenas. Whole-genome sequencing (WGS) is the current gold standard for SARS-CoV-2 variant identification; however, broad WGS adoption is hampered by financial restrictions and requirements for specialized equipment and bioinformatics expertise. While these limitations may be mitigated through centralized high-volume testing, the consequent increased turnaround time for batching, data deconvolution, and sequence analysis exceeds what is clinically actionable for most patient assessments. A simple, on-site reflex test of positive specimens for variants is a practical solution.

### BRIEF SUMMARY

In accordance with an aspect of the disclosure, provided herein are compositions for amplifying a SARS-COV-2 nucleic acid, the compositions comprising at least one set of nucleic acid primers (primer set) selected from: (i) a first primer set comprising a first nucleic acid at least 15 nucleotides in length and having at least 85% sequence identity to SEQ ID NO: 1, and a second nucleic acid at least 15 nucleotides in length and having at least 85% sequence identity to SEQ ID NO: 2; (ii) a second primer set comprising a first nucleic acid at least 15 nucleotides in length and having at least 85% sequence identity to SEQ ID NO: 14, and a second nucleic acid at least 15 nucleotides in length and having at least 85% sequence identity to SEQ ID NO: 15; (iii) a third primer set comprising a first nucleic acid at least 15 nucleotides in length and having at least 85% sequence identity to SEQ ID NO: 20, and a second nucleic acid at least 15 nucleotides in length and having at least 85% sequence identity to SEQ ID NO: 21; (iv) a fourth primer set comprising a first nucleic acid at least 15 nucleotides in length and having at least 85% sequence identity to SEQ ID NO: 16, and a second nucleic acid at least 15 nucleotides in length and having at least 85% sequence identity to SEQ ID NO: 17; (v) a fifth primer set comprising a first nucleic acid at least 15 nucleotides in length and having at least 85% sequence identity to SEQ ID NO: 22, and a second nucleic acid at least 15 nucleotides in length and having at least 85% sequence identity to SEQ ID NO: 23; (vi) a sixth primer set comprising a first nucleic acid at least 15 nucleotides in length and having at least 85% sequence identity to SEQ ID NO: 9, a second nucleic acid at least 15 nucleotides in length and having 85% sequence identity to SEQ ID NO: 10, and a third nucleic acid consisting of up to 15 nucleotides and having at least 85% sequence identity to SEQ ID NO: 11; (vii) a seventh primer set comprising a first nucleic acid at least 15 nucleotides in length and having at least 85% sequence identity to SEQ ID NO: 9, a second nucleic acid at least 15 nucleotides in length and having 85% sequence identity to SEQ ID NO: 12, and a third nucleic acid at least 15 nucleotides in length and having 85% sequence identity to SEQ ID NO: 13; and (viii) an eighth primer set comprising a first nucleic acid at least 15 nucleotides in length and having at least 85% sequence identity to SEQ ID NO: 9, a second nucleic acid at least 15 nucleotides in length and having at least 85% sequence identity to SEQ ID NO: 18, and a third nucleic acid at least 15 nucleotides in length and having 85% sequence identity to SEQ ID NO: 19.

In various aspects, the composition provided may comprise at least two sets, at least three sets, at least four sets, at least five sets, at least six sets, at least seven sets, or all eight sets of nucleic acids selected from (i), (ii), (iii), (iv), (v), (vi), (vii), or (viii).

In various aspects, at least one nucleic acid in the composition is labeled with a fluorescent label. In various aspects, the nucleic acid in the composition is from about 15 to about 25 nucleotides in length (e.g., about 20 nucleotides in length).

In various embodiments, the compositions provided herein may further comprise a nucleic acid from a sample suspected of comprising a SARS-COV-2 virus. In various embodiments, the SARS-COV-2 virus is a variant comprising at least one insertion, deletion or single nucleotide polymorphism relative to a nucleic acid obtained from a wildtype SARS-COV-2 virus. In some embodiments, the variant is selected from alpha, beta, gamma, iota, lambda or omicron.

Also provided is a method for detecting a SARS-COV-2 variant, the method comprising: (a) amplifying a nucleic acid in a sample suspected of comprising the SARS-COV-2 variant, the variant comprising at least one insertion, deletion or single nucleotide polymorphism (SNP) as compared to a wildtype SARS-COV-2 nucleic acid, and (b) detecting an amplicon produced in (a); wherein amplifying the nucleic acid comprises: (i) performing a polymerase chain reaction using at least one set of nucleic acids (primer set), wherein the primer set amplifies a nucleic acid of the SARS-COV-2 variant comprising the insertion or deletion and the amplicon detected in (b) is larger or smaller than a corresponding amplicon generated from a wildtype SARS-COV-2 nucleic acid; and/or (ii) performing a polymerase chain reaction using a first nucleic acid primer, a second nucleic acid primer and third nucleic acid primer, wherein the first nucleic acid primer preferentially anneals to a nucleic acid comprising a mutant allele of the SNP, the second nucleic acid primer preferentially anneals to a nucleic acid comprising wildtype allele of the SNP, and the third nucleic acid allows for the amplification of a nucleic acid comprising the SNP.

In any of the methods provided herein, the insertion or deletion may be located in a recurrently deleted region 1 (RDR1), a recurrently deleted region 2 (RDR2), a recurrently deleted region 34 (RDR3_4), an open reading frame 1A (ORF1A), or an open reading frame 8 (ORF8) and/or wherein the single nucleotide polymorphism encodes an amino acid substitution in a protein of the SARS-COV-2 virus, the amino acid substitution selected from N501Y, L452R, or E484K according to reference SEQ ID NO: 51.

In certain aspects, the insertion or deletion is located in the RDR1 and the primer set in (i) comprises a first nucleic acid at least 15 nucleotides in length and comprising at least 85% sequence homology with SEQ ID NO: 1; and a second nucleic acid at least 15 nucleotides in length and comprising at least 85% sequence homology with SEQ ID NO: 2. In some embodiments, the insertion or deletion in the RDR1 comprises a 6 base pair deletion or a 6 base pair insertion.

In various aspects, the insertion or deletion may be located in the RDR2, and the primer set in (i) comprises a first nucleic acid at least 15 nucleotides in length and comprising at least 85% sequence homology with SEQ ID NO: 14; and a second nucleic acid at least 15 nucleotides in length and comprising at least 85% sequence homology with SEQ ID NO: 15. In various embodiments, the insertion or deletion in RDR2 comprises a 3 base pair deletion, a 9 base pair deletion, or a 3 base pair insertion.

In various aspects, the insertion or deletion is located in the RDR3_4 and the primer set in (i) comprises a first nucleic acid at least 15 nucleotides in length and comprising at least 85% sequence homology with SEQ ID NO: 20; and a second nucleic acid at least 15 nucleotides in length and comprising at least 85% sequence homology with SEQ ID NO: 21. In some embodiments, the insertion or deletion in RDR_3_4 comprises a 9 base pair deletion, a 6 base pair insertion or a 21 base pair insertion.

In various aspects, the insertion or deletion is located in the ORF1A and the primer set in (i) comprises a first nucleic acid at least 15 nucleotides in length and comprising at least 85% sequence homology with SEQ ID NO: 16; and a second nucleic acid at least 15 nucleotides in length and comprising at least 85% sequence homology with SEQ ID NO: 17. In various embodiments, the insertion or deletion in ORF1A comprises a 9 base pair deletion.

In various aspects, the insertion or deletion is located in the ORF8 and the primer set in (i) comprises a first nucleic acid at least 15 nucleotides in length and comprising at least 85% sequence homology with SEQ ID NO: 22; and a second nucleic acid at least 15 nucleotides in length and comprising at least 85% sequence homology with SEQ ID NO: 23. In various embodiments, the insertion or deletion in ORF8 comprises a 4 base pair insertion or a 6 base pair deletion.

In any of the methods described herein, the amplicon produced in (b) may be larger or smaller as compared to a corresponding amplicon produced by amplifying a wildtype SARS-COV-2 nucleic acid in the presence of the at least one primer pair and wherein the method further comprises identifying the SARS-CO-V-2 variant by detecting the larger or smaller amplicon as compared to a control sample comprising a wildtype SARS-COV-2 nucleic acid.

In any of the methods herein, the single nucleotide polymorphism may result in a N501Y amino acid substitution in the encoded protein, and the primer set in (ii) comprises a first nucleic acid at least 15 nucleotides in length and comprising at least 85% sequence homology with SEQ ID NO: 9; and a second nucleic acid at least 15 nucleotides in length and comprising at least 85% sequence homology with SEQ ID NO: 10, and a third nucleic acid at least 15 nucleotides in length and comprising at least 85% sequence homology with SEQ ID NO: 11.

In further methods provided herein, the single nucleotide polymorphism results in a E484K amino acid substitution in the encoded protein, and the primer set in (ii) comprises a first nucleic acid at least 15 nucleotides in length and having at least 85% sequence identity to SEQ ID NO: 9, a second nucleic acid at least 15 nucleotides in length and having 85% sequence identity to SEQ ID NO: 12, and a third nucleic acid at least 15 nucleotides in length and having 85% sequence identity to SEQ ID NO: 13.

In various methods, the single nucleotide polymorphism may result in a L452R/Q amino acid substitution (e.g., L452R or L452Q substitution) in the encoded protein, and the primer set in (ii) comprises a first nucleic acid at least 15 nucleotides in length and having at least 85% sequence identity to SEQ ID NO: 9, a second nucleic acid at least 15 nucleotides in length and having at least 85% sequence identity to SEQ ID NO: 18, and a third nucleic acid at least 15 nucleotides in length and having 85% sequence identity to SEQ ID NO: 19.

In any of the methods provided herein, the first nucleic acid in (ii) may be labeled with a first fluorescent marker and the second nucleic acid in (ii) may be labeled with a second fluorescent marker, wherein the first and second fluorescent markers are independently detectable in a single sample and wherein the method further comprises identifying the SARS-COV-2 variant when a ratio of the first fluorescent marker to the second fluorescent marker exceeds a threshold.

In various aspects, any of the methods provided herein may comprise amplifying one or more the nucleic acids in a sample suspected of comprising the SARS-COV-2 variant using at least one set, at least two sets, at least three sets, at least four sets, at least five sets, at least six sets, at least seven sets, or at least eight sets of nucleic acid primers (primer sets) selected from: (i) a first primer set to detect an insertion or deletion in RDR1 comprising a first nucleic acid at least 15 nucleotides in length and having at least 85% sequence identity to SEQ ID NO: 1, and a second nucleic acid at least 15 nucleotides in length and having at least 85% sequence identity to SEQ ID NO: 2; (ii) a second primer set to detect an insertion or deletion in RDR2 comprising a first nucleic acid at least 15 nucleotides in length and having at least 85% sequence identity to SEQ ID NO: 14, and a second nucleic acid at least 15 nucleotides in length and having at least 85% sequence identity to SEQ ID NO: 15; (iii) a third primer set to detect an insertion or deletion in RDR3_4 comprising a first nucleic acid at least 15 nucleotides in length and having at least 85% sequence identity to SEQ ID NO: 20, and a second nucleic acid at least 15 nucleotides in length and having at least 85% sequence identity to SEQ ID NO: 21; (iv) a fourth primer set to detect an insertion or deletion in ORF1 a comprising a first nucleic acid at least 15 nucleotides in length and having at least 85% sequence identity to SEQ ID NO: 16, and a second nucleic acid at least 15 nucleotides in length and having at least 85% sequence identity to SEQ ID NO: 17; (v) a fifth primer set to detect an insertion or deletion in ORF8 comprising a first nucleic acid at least 15 nucleotides in length and having at least 85% sequence identity to SEQ ID NO: 22, and a second nucleic acid at least 15 nucleotides in length and having at least 85% sequence identity to SEQ ID NO: 23; (vi) a sixth primer set to detect an N501Y SNP comprising a first nucleic acid at least 15 nucleotides in length and having at least 85% sequence identity to SEQ ID NO: 9, a second nucleic acid at least 15 nucleotides in length and having 85% sequence identity to SEQ ID NO: 10, and a third nucleic acid at least 15 nucleotides in length and having at least 85% sequence identity to SEQ ID NO: 11; (vii) a seventh primer set to detect an E484K SNP comprising a first nucleic acid at least 15 nucleotides in length and having at least 85% sequence identity to SEQ ID NO: 9, a second nucleic acid at least 15 nucleotides in length and having 85% sequence identity to SEQ ID NO: 12, and a third nucleic acid at least 15 nucleotides in length and having 85% sequence identity to SEQ ID NO: 13; and (viii) an eighth primer set to detect a L452R SNP comprising a first nucleic acid at least 15 nucleotides in length and having at least 85% sequence identity to SEQ ID NO: 9, a second nucleic acid at least 15 nucleotides in length and having at least 85% sequence identity to SEQ ID NO: 18, and a third nucleic acid at least 15 nucleotides in length and having 85% sequence identity to SEQ ID NO: 19; and wherein the SARS-COV-2 variant is identified based on the presence or absence of the insertion or deletion in RDR1, RDR2, RDR3_4, ORF1a, ORF8, or the presence or absence of the N501Y, NE484K, or L452R SNPs.

In various methods herein, the SARS-COV-2 variant may be identified as an alpha variant, a beta variant, a gamma variant, an iota variant, a lambda variant, a delta variant, or an omicron variant.

For example, the SARS-COV-2 variant may be identified as an alpha variant when a 6 basepair deletion is detected in RDR1, a 3 base pair deletion is detected in RDR2, a 9 base pair deletion is detected in ORF1A, and an N501Y single nucleotide polymorphism is detected.

As another example, the SARS-COV-2 variant may be identified as a beta variant when a 9 base pair deletion is detected in RDR3_4, a 9 base pair deletion is detected in ORF1A, and N501Y and E484K single nucleotide polymorphisms are detected.

As yet another example, the SARS-COV-2 variant may be identified as a gamma variant when a 4 base pair insertion is detected in ORF8, a 9 base pair deletion is detected in ORF1A, and N501Y and E484K single nucleotide polymorphisms are detected.

As another example, the SARS-COV-2 variant may be identified as an iota variant when a 9 base pair deletion is detected in ORF1A, and an E484K single nucleotide polymorphism is detected.

In another example, the SARS-COV-2 variant may be identified as a lambda variant when a 9 base pair deletion is detected in ORF1A, a 21 base pair deletion is detected in RDR3_4 and amplification of a mutant allele at position L452 is observed.

In another example, the SARS-COV-2 variant may be identified as an omicron variant when a 6 base pair deletion is detected in RDR1, a 9 base pair deletion is detected in RDR2, a 6 base pair insertion is detected in RDR3_4, a 9 base pair deletion is detected in ORF1A, and an N501Y single nucleotide polymorphism is detected.

As another example, the SARS-COV-2 variant may be identified as a mu variant when a 3 base pair insertion is detected in RDR2, and an N501Y and E484K single nucleotide polymorphisms are detected.

As yet another example, the SARS-COV-2 variant may be identified as a delta variant when a 6 base pair deletion is detected in RDR2⁻, a 6 base pair deletion is detected in ORF8, a L452R single nucleotide polymorphism is detected, and amplification of a wildtype allele at position E484 is reduced without an increase in amplification of a mutant allele.

In any of the methods provided herein, detecting the amplicon in (b) may comprise capillary electrophoresis or fragment analysis (e.g., Sanger Sequencing). In various aspects, amplifying the nucleic acid in (a) and/or detecting the amplicon in (b) can comprise using real-time PCR (RT-PCR).

In any of the methods provided herein, more than one SARS-COV-2 variant (e.g., two or more distinct/different variants) may be identified in a single sample.

In any of the methods provided herein, the sample may be selected from a nasopharyngeal swab sample, an oropharyngeal sample, a nasal aspirate sample, a nasal or mid-turbinate swab, a nasal aspirate sample, a nasal wash sample, a throat swab sample, a bronchoalveolar lavage sample, a bronchial aspirate sample, a bronchial wash sample, an endotracheal aspirate, an endotracheal wash sample, a tracheal aspirate, a nasal secretion sample, a mucus sample, a sputum sample, a lung tissue samples, a urine sample, a saliva sample, and a fecal sample.

Also provided are methods of generating a primer for detecting a single nucleotide polymorphism (SNP) in a nucleic acid, the method comprising: (a) providing a first nucleic acid sequence complementary to a nucleic acid comprising a mutant allele of the SNP, wherein the first nucleic acid sequence comprises a terminal 3′ nucleotide complementary to either the mutant or the wildtype nucleotide at the location of the SNP, (b) substituting an adenine (A) for a thymine (T) (T>A substitution) one base pair upstream (5′ direction) of the terminal 3′ nucleotide complementary to the mutant or wildtype nucleotide at the location of the SNP to generate a mutant primer sequence or a wildtype primer sequence, and (c) generating a mutant primer comprising the mutant primer sequence and/or a wildtype primer comprising the wildtype primer sequence; wherein binding of the mutant primer to a wildtype allele or the binding of the wildtype primer to a mutant allele is destabilized as compared to a mutant primer or wildtype primer without the T>A substitution.

In another aspect, another method of generating a primer for detecting a single nucleotide polymorphism in a nucleic acid is provided, the method comprising: (a) providing a first nucleic acid sequence complementary to a nucleic acid comprising a mutant allele of the SNP, wherein the first nucleic acid sequence comprises a terminal 3′ nucleotide complementary to the mutant nucleotide at the location of the SNP, (b) generating a mutant primer sequence by substituting an adenine for a cytosine (C>A substitution) two base pairs upstream (5′ direction) of the terminal 3′ nucleotide that is complementary to the mutant allele, and (c) preparing an isolated nucleic acid primer (mutant primer) comprising the mutant primer sequence, wherein the mutant primer binds the mutant allele with a higher specificity than an equivalent primer without the C>A substitution.

Also provided herein are isolated nucleic acid primers, wherein the nucleic acid primers may comprise nucleotide sequences consisting of any one of SEQ ID NOs: 1 to 50. For example, in some aspects, the isolated nucleic acid primer comprises a nucleotide sequence consisting of any one of SEQ ID NOs: 1, 2, 14, 15, 16, 17, 9, 10, 11, 12, 13, 18, 19, 20, 21, 22, and 23. In another aspect, the isolated nucleic acid primer can comprise a nucleotide sequence consisting of any one of SEQ ID NOs: 10, 11, 24, 25, 26, 27, 28, 29, 30, 31, and 32. In yet another aspect, the isolated nucleic acid primer can comprise a nucleotide sequence consisting of any one of SEQ ID NOs: 12, 13, 33, 34, 35, 36, 37, and 38. As another example, the isolated nucleic acid primer can comprise of a nucleotide sequence consisting of any one of SEQ ID NOs: 18, 19, 39, 40, 41, 42, 43, 44, 45, 46, 47, 48, 49, and 50. In various embodiments, the isolated nucleic acid primer may have a length of about 20 to about 25 nucleotides. For example, the isolated nucleic acid primer may have a length of 20 nucleotides.

### BRIEF DESCRIPTION OF THE DRAWINGS

Embodiments of the present inventive concept are illustrated by way of example in which like reference numerals indicate similar elements and in which:

FIGS. 1A-11. FIG. 1A: Genetic variations of the SARS-CoV-2 B.1.1.7 (UK) Variant of Concern. Black arrows indicate primer binding sites. Green stars indicating fluorescent dye (FAM) primer labels. FIG. 1B: Routine clinical workflow and timetable of the SARS-CoV-2 fragment analysis assay. FIG. 1C: Comparison of analytical and operational characteristics of real time PCR (RT-qPCR), fragment analysis by capillary electrophoresis and WGS (whole genome sequencing). FIG. 1D: Expected deletions that should be present in the SARS-CoV-2 B.1.1.7 strain compared to wild type as visualized by fragment analysis. FIG. 1E: Electrophoretogram representative of two patients with WT and B.1.1.7 SARS-CoV-2 infections. FIG. 1F: Estimated prevalence of presumptive B.1.1.7 per week with number of specimens tested below. FIGS. 1G-1H: Integrated genome viewer visualization of the whole genome sequencing reads confirming the presence of the ORF1A:3675_3677del (FIG. 1G), S:69_70del (FIG. 1H), and S:144del (I) initially identified by capillary electrophoresis.

FIGS. 2A-2K. FIG. 2A: Expected deletions present in B.1.429 (California) strain compared to wild type SARS-CoV-2. FIG. 2B: Electrophoretogram representative of two patients with wild type and B.1.429 SARS-CoV-2 infections. FIG. 2C: Genetic variation of the SARS-CoV-2 B.1.429 strain. Black arrows indicate the primer binding sites for S144 target primers. The red line indicates the location of the W152C genetic variant overlapping with the S114 reverse primer. FIG. 2D: Detailed view of the variant causing W152C and proposed alternative primer binding site, which accounts for the 3-nucleotide insertion observed. FIG. 2E: Expected deletions present in P.1 and B.1.351 strains compared to WT (wild type) SARS-CoV-2. FIG. 2F: Electrophoretograms representative of two patients: one with wild type SARS-CoV-2 infection, the second with a virus that exhibits a nine base pair deletion in ORF1A. FIG. 2G: Genetic variation of the SARS-CoV-2 P.1 strain. Integrated genome viewer visualization of the whole genome sequencing reads confirming the presence of the SW152C variant characteristic of the B.1.429 strain (FIG. 2H) and ORF1A:3675_3677del (FIG. 2I), which is found in each Variant of Concern. Lack of the N501Y (FIG. 2J) and E484K (FIG. 2K) mutations precludes classification of this SARS-CoV-2 strain as P.1/B.1.351.

FIGS. 3A-3F. Fragment analysis identifies the SARS-CoV-2 Alpha Variant of Concern. FIG. 3A: Genetic variations of the SARS-CoV-2 Alpha VOC. Black arrows indicate primer binding sites. Stars indicating fluorescent dye (Blue-FAM, Green-HEX) primer labels. FIG. 3B: Expected deletions present in the SARS-CoV-2 Alpha variant compared to wild type as visualized by fragment analysis. FIG. 3C: Electrophoretogram representative of two patients with WT and Alpha SARS-CoV-2 infections. Integrated genome viewer visualization of the whole genome sequencing reads confirming the presence of the ORF1A:3675_3677del (FIG. 3D), S:69_70del (FIG. 3E), and S:144del (FIG. 3F) initially identified by capillary electrophoresis.

FIGS. 4A-4E. Detection of Iota (B.1.526, New York) SARS-CoV-2 variant by fragment analysis. FIG. 4A: Expected deletions present in Gamma, (P.1, Brazil), Beta (B.1.351, South Africa), and Iota (B.1.526, New York) VOCs compared to wild type. FIG. 4B: Electrophoretograms representative of clinical wild type SARS-CoV-2 infection and one with SARS-CoV-2 exhibiting a nine-base pair deletion in ORF1A characteristic of Beta/Gamma/Iota. Integrated genome viewer (IGV) visualization of the whole genome sequencing reads confirming the presence of the ORF1A:de13675_3677 (FIG. 4C), which is found in Beta/Gamma/Iota. Lack of the N501Y (FIG. 4D) and presence of the E484K (FIG. 4E) mutation along with other mutations led to classification of this SARS-CoV-2 strain as Iota.

FIGS. 5A-5G. Allele-specific primers for S:N501Y and S:E484K. FIG. 5A: Scheme for designing allele-specific primers with destabilizing mutations. FIG. 5B: Electrophoretograms of four combinations of E484WT and E484K labeled primers with (FIG. 5C) graphs quantifying fluorescence intensity in WT specimens, (FIG. 5D) E484K mutated specimens and (FIG. 5E) the ratio of the two. FIG. 5F: Electrophoretogram of signal from N501 and N501Y allele-specific primers. FIG. 5G: Graphical representation of FAM and HEX signal in response to WT and N501Y specimens.

FIGS. 6A-6F. Allele-specific primers for S:L452R. FIG. 6A: Electrophoretograms of four combinations of L452WT and L452R labeled primers. (FIG. 6B, FIG. 6C) RT-qPCR curves of various L452WT and L452R specific primers binding to either WT or mutant sequences. Arrows demonstrate how the same primer is less reactive to WT sequence (FIG. 6C) or how mutant primers are less reactive to WT sequence (FIG. 6B). Melting curves are shown to the right to confirm production of a single PCR product for each case. Graphs quantifying fluorescence intensity in WT specimens (FIG. 6D) and a B.1.429 mutated specimen positive for S:L452R (FIG. 6C), and the ratio of the two (FIG. 6F).

FIGS. 7A-7C. Epidemiologic data derived from mutational signatures. FIG. 7A: Epidemiologic data produced from the fragment analysis and WGS. FIG. 7B: Mutational signatures of the major variants of concern as detected by fragment analysis. FIG. 7C: Electrophoretograms of the core set of targets for the Gamma, Delta, and Omicron variants.

FIGS. 8A-8C. Expected and actual mutational signatures of Lambda and Mu variants. FIG. 8A: Electrophoretogram of Lambda variant (C.37) characterized by ORF1A A9 bp, RDR3-4 Δ21 bp, and L452Mut. FIG. 8B: Electrophoretogram of Mu variant (B.1.621) characterized by RDR2 3 bp insertion, N501Y, and E484K. FIG. 8C: Expected mutational signature of lambda and mu variants.

FIG. 9 shows exemplary scatter plots showing the Ct threshold, on-target and off-target specificity and melting temperature of exemplary primers.

FIG. 10 is a bar graph depicting Ct cycles relative to annealing temperatures for various EEK primers.

FIG. 11 shows bar graphs of a wildtype primer binding to mutant (L452R) or wildtype SARS-COV-2 alleles.

FIG. 12 shows bar graphs of a mutant primer binding to mutant (L452R) or wildtype SARS-COV-2 alleles.

FIG. 13 shows optimization scans to determine annealing temperatures for primer use.

FIG. 14 is a line plot of the limit of detection for each primer.

FIG. 15 is a plot showing percent positive results relative to CT threshold for the CoVarScan and WGS (whole genome sequencing).

FIG. 16. Capillary electrophoresis result of mutations in selected targets of CoVarScan. A wild type variant is shown on top for reference, Omicron (BA.1) in middle panel, and BA.2's mutational signature is visible in the bottom panel.

FIGS. 17A-17B. FIG. 17A: Case summary of first Delta-Omicron co-infection with dates of COVID-19 testing and variant analysis (top), vaccination+anti-viral treatment (row two), immunosuppression therapies (row three), and monoclonal antibody therapy/COVID serology (bottom). C/I: Casirivimab/Imdevimab, C/T: Cilgavimab/Tixagevimab, V1,V2, V3: Vaccination 1, 2, and 3, Rem: Remdesivir, ND: not detected, AU: arbitrary units. FIG. 17B: Summary of 5 cases of co-infection for both Delta-Omicron and BA.1-BA.2.

FIGS. 18A-D. FIG. 18A: Electrophoretogram of the ORF1A, Spike region 1 and Spike region 2 sites where different sized amplicons indicative of Delta (top) Omicron (middle) or co-infection (bottom) are present. FIG. 18B: Next-generation sequencing reads were visualized in the Integrated Genome Viewer with read direction colored red or blue. Deletions are indicated by a bar, single nucleotide variants have the variant nucleotide present, and insertions are highlighted by a purple box. Schematic illustrating the expected location of fragments for variants under normal (FIG. 18C), mixed or recombined conditions (FIG. 18D).

FIGS. 19A-19C. FIG. 19A: Electrophoretogram of ORF1A, Spike region 1, Spike region 2, and Spike region 3-4 sites where different sized amplicons indicative of Omicron (red) and BA.2 (green) co-infection are present. FIG. 19B: Schematic illustrating the expected location of fragments for variants under normal, mixed or recombined conditions. FIG. 19C: Next-generation sequencing reads were visualized in the Integrated Genome Viewer with read direction colored red or blue. Deletions are indicated by a bar, single nucleotide variants have the variant nucleotide present, and insertions are highlighted by a purple box.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, at least in part on, the development of a new platform for identifying SARS-COV-2 variants. With the emergence of new variants, many labs will need to monitor for their spread. It is difficult to change the assays of commercial platforms that are used by most laboratories, and the definitive method of detection (Whole Genome sequencing by Next Generation Sequencing) is cumbersome, expensive, and requires complex data analysis. Methods, and primers, described herein can be implemented, quickly, easily, and cheaply in almost any molecular lab.

Here, a rapid, cost-effective, and scalable method utilizing fragment analysis by capillary electrophoresis is described to detect known SARS-CoV-2 variants in a routine clinical setting. In this procedure, extracted RNA is amplified to create fluorescently marked amplicons that are separated by capillary electrophoresis on a Sanger sequencer. Variants are then identified by their abnormal amplicon sizes. By measuring the resultant changes in fragment length, this fragment analysis assay permits a broader range of variant detection compared to primer-probe based RT-qPCR. The reagent and instrumentation requirements for this assay are modest: fluorescently labeled primers, a thermocycler, and Sanger sequencer. Moreover, the time requirement (4 hours), simplicity, and use of 96-well format support the assay's scalability. Amplicons can be designed up to 500 bp in length with up to four fluorescent labels, making the assay adaptable to detect other SARS-CoV-2 variants as they are identified (potential to multiplex 20-40 targets). This assay provides a mechanism for routine clinical laboratories to screen positive specimens for SARS-CoV-2 variants of concern (VOC) that can be easily and broadly implemented with limited financial burden or bioinformatics requirements.

### 1. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all value from 16.6 degrees to 150 degrees. For example, they can refer to less than or equal to +5%, such as less than or equal to +2%, such as less than or equal to 1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

### I. Nucleic Acid Primers

In various embodiments, nucleic acid primers are provided herein that can be used according to methods in the art and described herein below to identify samples containing SARS-Co-V-2 variants.

Several SARS CoV-2 variants have been identified and continue to be identified. Some exemplary variants are described in the tables below. In each table, the mutation is described as the nucleic acids deleted in the mutant allele relative to the full-length SARS-CoV-2 genome (NCBI Reference Sequence NC_045512.2 (Severe acute respiratory syndrome coronavirus 2 isolate Wuhan-Hu-1, complete genome, SEQ ID NO: 51)).

Table 1A, below describes variants and mutations covering Alpha and Beta variants.

TABLE 1A

| Target | Mutation | CoVarScan Signature |
|---|---|---|
| S: Del69_70 | 21765_21770del | 6 base pair deletion in RDR1 |
| S: Del144 | 21991_21993del | 3 base pair deletion in RDR2 |
| ORF1A: 9 bp | 11288_11296del | 9 base pair deletion in ORFA1A |
| del | | |

Table 1B, below, includes the mutations covered in the previous table and also describes additional mutations covering Gamma, Iota, Lambda, Omicron, Epsilon and Mu variants.

TABLE 1B

| Mutation | | Exemplary | CoVarScan | |
|---|---|---|---|---|
| Category | Target | Target | Signature | Variant |
| Insertion/Deletion | RDR1 | S69/70 | 6 base pair | Alpha, |
| | | | deletion | Omicron |
| Insertion/Deletion | RDR2 | S144 | 3 base pair | Alpha |
| | | | deletion | |
| | | | 9 base pair | Omicron |
| | | | deletion | |
| | | | 3 base pair | Mu |
| | | | insertion | |
| | | | 6 base pair | Delta |
| | | | deletion | |
| Insertion/Deletion | RDR3_4 | | 9 base pair | Beta |
| | | | deletion | |
| | | | 6 base pair | Omicron |
| | | | insertion | |
| Insertion/Deletion | ORF1A | ORFA1A | 9 base pair | Alpha, |
| | | | deletion | Beta, |
| | | | | Gamma, |
| | | | | Iota, |
| | | | | Lambda, |
| | | | | Omicron |
| Insertion/Deletion | ORF8 | | 4 base pair | Gamma |
| | | | insertion | |
| | | | 6 base pair | Delta |
| | | | deletion | |
| SNP | N501 | | N501Y | Alpha, |
| | | | | Beta, |
| | | | | Gamma, |
| | | | | Omicron, |
| | | | | Mu |
| SNP | E484 | | E484K | Beta, |
| | | | | Gamma, |
| | | | | Iota, Mu |
| SNP | L452 | | L452R | Delta |
| | | | L482Q | Lambda |

Surprisingly, it has been discovered that many mutations, including those in new and emerging strains of the virus, tend to cluster around 8 hotspots. For example, insertions and deletions have been found in the following regions: RDR (repeatedly deleted region), and ORF (open reading frame). And as another example, common single nucleotide polymorphisms have been identified (e.g., at locations encoding for amino acids N501, E484 and L452 in the spike protein of the native SARS-COV-2 variant, SEQ ID NO: 52). Together, as described further below, these 8 regions provide a unique signature that may be used to identify any known variant along with any closely related variants that arise.

Accordingly, in various embodiments a nucleic acid primer having a nucleic acid sequence complementary to a portion of RDR1 (e.g., S69/70), RDR2 (e.g., S144), RGR3_4, ORFA1A, ORF8, S: N501Y, S: E484K, or S: L452R allele is provided.

In various embodiments, the nucleic acid primer is complementary to a portion of a SARS-COV-2 nucleic acid spanning a region that includes an insertion or deletion relative to wildtype. For example, in various embodiments, the nucleic acid primer is complementary to a portion of the RDR1 (S69/70) allele and comprises a nucleic acid sequence having at least 80%, at least 85%, at least 90%, at least 95%, at least 96%, at least 97%, at least 98%, at least 99%, or 100% identity to SEQ ID NO: 1 or 2. In various embodiments, the nucleic acid primer is complementary to a portion of the S144 allele and comprises a nucleic acid sequence having at least 80%, at least 85%, at least 90%, at least 95%, at least 96%, at least 97%, at least 98%, at least 99%, or 100% identity to SEQ ID NO: 3 or 4. In various embodiments, the nucleic acid primer is complementary to a portion of the S144 allele and comprises a nucleic acid sequence having at least 80%, at least 85%, at least 90%, at least 95%, at least 96%, at least 97%, at least 98%, at least 99%, or 100% identity to SEQ ID NO: 14 or 15. In various embodiments, the nucleic acid primer is complementary to a portion of the ORFA1A allele and comprises a nucleic acid sequence having at least 80%, at least 85%, at least 90%, at least 95%, at least 96%, at least 97%, at least 98%, at least 99%, or 100% identity to SEQ ID NO: 5 or 6. In various embodiments, the nucleic acid primer is complementary to a portion of the ORFA1A allele and comprises a nucleic acid sequence having at least 80%, at least 85%, at least 90%, at least 95%, at least 96%, at least 97%, at least 98%, at least 99%, or 100% identity to SEQ ID NO: 16 or 17. In various embodiments, the nucleic acid primer is complementary to a portion of the ORF8 allele and comprises a nucleic acid sequence having at least 80%, at least 85%, at least 90%, at least 95%, at least 96%, at least 97%, at least 98%, at least 99%, or 100% identity to SEQ ID NO: 22 or 23. In various embodiments, the nucleic acid primer is complementary to a portion of the RDR3_4 allele and comprises a nucleic acid sequence having at least 80%, at least 85%, at least 90%, at least 95%, at least 96%, at least 97%, at least 98%, at least 99%, or 100% identity to SEQ ID NOs: 20 or 21. Any of the primers described herein may be at least 15 nucleotides (e.g., at least 15, at least 16, at least 17, at least 18, at least 19, or at least 20 nucleotides. In various aspects, the primers may be up to about 30, up to about 29, up to about 28, up to about 27, up to about 26, or up to about 25 nucleotides in length. For example, the primers may be about 15 to about 25 nucleotides in length. In various aspects, the primers may be 20 nucleotides in length. Any of these primers may comprise one or more modifications to improve stability at the target allele (mutant or wildtype) and/or to decrease stability at the non-targeted allele (mutant or wildtype). Methods for optimizing these primers are described further, below.

Another variant of SARS CoV-2 (spike protein N501Y) is common to 4 independent emerging strains of SARS-CoV-2 (UK (Alpha), South Africa (Beta), Brazil (Gamma), and Ohio (Delta)). This mutation is present in the binding site where the spike protein binds ACE2 receptor to enter into lung cells. Studies have shown the mutation increases the binding strength to increase infectivity. This example of convergent evolution indicates this variant is important for monitoring known and emerging SARS-CoV-2 variants. Additional common mutations in the same spike protein (e.g., E484K, L452R/Q) have been reported. Accordingly, primers are required to identify variants as having one or more of these mutations.

In further embodiments, the nucleic acid primer is complementary to a region comprising a single nucleotide polymorphism in a variant relative to a wild-type (native) SARS-COV-2 nucleic acid. Exemplary single nucleotide polymorphisms discovered in SARS-COV-2 variants encode for the following amino acid substitutions: N501Y, E484K, and L452R/Q. Accordingly, in some embodiments, the nucleic acid primer is complementary to a portion of a nucleic acid encoding for an amino acid at a position equivalent to position 501, 484, or 452 in a wildtype spike protein of the SARS-COV-2 virus (SEQ ID NO: 52). In some embodiments, the nucleic acid primer comprises a terminal nucleotide complementary to the single nucleotide polymorphism in a mutant allele (e.g., a mutant forward primer). In some embodiments, the nucleic acid primer comprises a terminal nucleotide complementary to the single nucleotide polymorphism in a wildtype allele (e.g., a wildtype forward primer). In some embodiments, the nucleic acid primer is complementary to a portion of the targeted allele not comprising the single nucleotide polymorphism (e.g., a common reverse polymer). Any of these primers may comprise one or more modifications to improve stability at the target allele (mutant or wildtype) and/or to decrease stability at the non-targeted allele (mutant or wildtype). Methods for optimizing these primers are described further, below.

In various embodiments, a nucleic acid primer is provided that is complementary to a region comprising a SNP encoding for an amino acid equivalent to position 501 in the wildtype SARS-COV-2 spike protein (SEQ ID NO: 52). In various embodiments, the nucleic acid primer comprises a nucleic acid sequence having at least 80%, at least 85%, at least 90%, at least 95%, at least 96%, at least 97%, at least 98%, at least 99%, or 100% identity to any one of SEQ ID NOs: 9 to 11. In some embodiments, the nucleic acid primer comprises a nucleic acid sequence having at least 80%, at least 85%, at least 90%, at least 95%, at least 96%, at least 97%, at least 98%, at least 99%, or 100% identity to SEQ ID NOs: 9. In some embodiments, the nucleic acid primer comprises a nucleic acid sequence having at least 80%, at least 85%, at least 90%, at least 95%, at least 96%, at least 97%, at least 98%, at least 99%, or 100% identity to SEQ ID NOs: 10. In some embodiments, the nucleic acid primer comprises a nucleic acid sequence having at least 80%, at least 85%, at least 90%, at least 95%, at least 96%, at least 97%, at least 98%, at least 99%, or 100% identity to SEQ ID NOs: 11. Any of the primers described herein may be at least 15 nucleotides (e.g., at least 15, at least 16, at least 17, at least 18, at least 19, or at least 20 nucleotides. In various aspects, the primers may be up to about 30, up to about 29, up to about 28, up to about 27, up to about 26, or up to about 25 nucleotides in length. For example, the primers may be about 15 to about 25 nucleotides in length. In various aspects, the primers may be 20 nucleotides in length. Any of these primers may comprise one or more modifications to improve stability at the target allele (mutant or wildtype) and/or to decrease stability at the non-targeted allele (mutant or wildtype). Methods for optimizing these primers are described further, below.

In various embodiments, a nucleic acid primer is provided that is complementary to a region comprising a SNP encoding for an amino acid equivalent to position 484 in the wildtype SARS-COV-2 spike protein (SEQ ID NO: 52). In various embodiments, the nucleic acid primer comprises a nucleic acid sequence having at least 80%, at least 85%, at least 90%, at least 95%, at least 96%, at least 97%, at least 98%, at least 99%, or 100% identity to any one of SEQ ID NOs: 9, 12 or 13. In some embodiments, the nucleic acid primer comprises a nucleic acid sequence having at least 80%, at least 85%, at least 90%, at least 95%, at least 96%, at least 97%, at least 98%, at least 99%, or 100% identity to SEQ ID NOs: 9. In some embodiments, the nucleic acid primer comprises a nucleic acid sequence having at least 80%, at least 85%, at least 90%, at least 95%, at least 96%, at least 97%, at least 98%, at least 99%, or 100% identity to SEQ ID NOs: 12. In some embodiments, the nucleic acid primer comprises a nucleic acid sequence having at least 80%, at least 85%, at least 90%, at least 95%, at least 96%, at least 97%, at least 98%, at least 99%, or 100% identity to SEQ ID NOs: 13. Any of the primers described herein may be at least 15 nucleotides (e.g., at least 15, at least 16, at least 17, at least 18, at least 19, or at least 20 nucleotides. In various aspects, the primers may be up to about 30, up to about 29, up to about 28, up to about 27, up to about 26, or up to about 25 nucleotides in length. For example, the primers may be about 15 to about 25 nucleotides in length. In various aspects, the primers may be 20 nucleotides in length. Any of these primers may comprise one or more modifications to improve stability at the target allele (mutant or wildtype) and/or to decrease stability at the non-targeted allele (mutant or wildtype). Methods for optimizing these primers are described further, below.

In various embodiments, a nucleic acid primer is provided that is complementary to a region comprising a SNP encoding for an amino acid equivalent to position 452 in the wildtype SARS-COV-2 spike protein (SEQ ID NO: 52). In various embodiments, the nucleic acid primer comprises a nucleic acid sequence having at least 80%, at least 85%, at least 90%, at least 95%, at least 96%, at least 97%, at least 98%, at least 99%, or 100% identity to any one of SEQ ID NOs: 9, 18 or 19. In some embodiments, the nucleic acid primer comprises a nucleic acid sequence having at least 80%, at least 85%, at least 90%, at least 95%, at least 96%, at least 97%, at least 98%, at least 99%, or 100% identity to SEQ ID NOs: 9. In some embodiments, the nucleic acid primer comprises a nucleic acid sequence having at least 80%, at least 85%, at least 90%, at least 95%, at least 96%, at least 97%, at least 98%, at least 99%, or 100% identity to SEQ ID NOs: 18. In some embodiments, the nucleic acid primer comprises a nucleic acid sequence having at least 80%, at least 85%, at least 90%, at least 95%, at least 96%, at least 97%, at least 98%, at least 99%, or 100% identity to SEQ ID NOs: 19. Any of the primers described herein may be at least 15 nucleotides (e.g., at least 15, at least 16, at least 17, at least 18, at least 19, or at least 20 nucleotides. In various aspects, the primers may be up to about 30, up to about 29, up to about 28, up to about 27, up to about 26, or up to about 25 nucleotides in length. For example, the primers may be about 15 to about 25 nucleotides in length. In various aspects, the primers may be 20 nucleotides in length. Any of these primers may comprise one or more modifications to improve stability at the target allele (mutant or wildtype) and/or to decrease stability at the non-targeted allele (mutant or wildtype). Methods for optimizing these primers are described further, below.

For ease of reference, SEQ ID NOs 1 to 6 and 9 to 23 are provided in Table 2. Also provided are exemplary primers comprising SEQ ID NOs 7 or 8 that can be used to identify the presence of any SARS-CoV-2 virus (N1).

TABLE 2

| | | SEQ ID |
|---|---|---|
| Name | Sequence | NO: |
| S69/70_F: | 5′-FAM-CGTGGTGTTTATTACCCTGACAAAG-3′ | 1 |
| S69/70_R: | 5′-TCAGTGGAAGCAAAATAAACACCAT-3′ | 2 |
| S144_F: | 5′-FAM-ACGCTACTAATGTTGTTATTAAAGTCT-3′ | 3 |
| S144_R: | 5′-TCTGAACTCACTTTCCATCCAACT-3′ | 4 |
| ORF1A_F: | 5′-FAM-TGCCTGCTAGTTGGGTGATG-3′ | 5 |
| ORF1A_R: | 5′-TGCTGTCATAAGGATTAGTAACACT-3′ | 6 |
| N1_F: | 5′-FAM-GACCCCAAAATCAGCGAAAT-3′ | 7 |
| N1_R: | 5′-TCTGGTTACTGCCAGTTGAATCTG-3′ | 8 |
| UTSW_Common_230_R | 5′-CAGTAGTGTCAGCAATGTCTCTGCC | 9 |
| UTSW_N501WT | 5′-FAM-CAATCATATGGTTTCCAACCCACTA | 10 |
| UTSW_N501Y_3′T | 5′-HEX-CAATCATATGGTTTCCAACCCATTT | 11 |
| UTSW_E484WT_3′G | 5′-FAM-GGTAGCACACCTTGTAATGGTGTCG | 12 |
| UTSW_E484K | 5′-HEX-CGGTAGCACACCTTGTAATGGTGTTA | 13 |
| Yale_144del_F: (RDR_F) | 5′-FAM-GAATTTGAATGATCCATTTTTGGGTG | 14 |
| UTSW_RDR2_R: | 5′-GGCTGAGAGACATATTCAAAGTGC | 15 |
| UTSW_ORF1A_F + 1: | 5′-FAM-TGCCTGCTAGTTGGGTGATG | 16 |
| UTSW_ORF1A_R + 5: | 5′-GTTCTTGCTGTCATAAGGATTAGTAACAC | 17 |
| UTSW_L452WT_3′G | 5′-FAM-CTAAGGTTGGTGGTAATTATAATTAGCT | 18 |
| UTSW_L452R | 5′-HEX-CTAAGGTTGGTGGTAATTATAATTACCG | 19 |
| UTSW_RDR3-4_F | 5′-FAM-GGACCTTGAAGGAAAACAGGG | 20 |
| UTSW_RDR3-4_R | 5′-TGAAGATAACCCACATAATAAGCTGC | 21 |
| UTSW_ORF8_F | 5′-FAM-GCCAGAAACCTAAATTGGGTAGT | 22 |
| UTSW_ORF8_R | 5′-CCCACTGCGTTCTCCATTCTG | 23 |

In addition to the primers described above, additional primers are also provided that may be used to target the described SNPs.

For example, nucleic acid primers complementary to an allele encoding for the N501Y SNP may comprises a nucleic acid sequence having at least 80%, at least 85%, at least 90%, at least 95%, at least 96%, at least 97%, at least 98%, at least 99%, or 100% identity to any one of SEQ ID NOs: 10, 11 or 24-32. As another example, nucleic acid primers complementary to an allele encoding for the N484Y allele may comprises a nucleic acid sequence having at least 80%, at least 85%, at least 90%, at least 95%, at least 96%, at least 97%, at least 98%, at least 99%, or 100% identity to any one of SEQ ID NOs: 12, 13, or 33-38. As another example, nucleic acid primers complementary to an allele encoding for the L452R allele may comprises a nucleic acid sequence having at least 80%, at least 85%, at least 90%, at least 95%, at least 96%, at least 97%, at least 98%, at least 99%, or 100% identity to any one of SEQ ID NOs: 18, 19, or 39-50. Any of the primers described herein may be at least 15 nucleotides (e.g., at least 15, at least 16, at least 17, at least 18, at least 19, or at least 20 nucleotides. In various aspects, the primers may be up to about 30, up to about 29, up to about 28, up to about 27, up to about 26, or up to about 25 nucleotides in length. For example, the primers may be about 15 to about 25 nucleotides in length. In various aspects, the primers may be 20 nucleotides in length.

For ease of reference, SEQ ID NOs: 24 to 50 are provided in Table 3 below. All of these primers may be combined with a suitable common reverse primer (e.g., SEQ ID NO: 9) according to the methods provided herein.

TABLE 3

| Primer Name | Sequence | SEQ ID NO: |
|---|---|---|
| N501_WT2TA | catatggtttccaacccacaa | 24 |
| N501y2TA | catatggtttccaacccacat | 25 |
| N501_Mut | caatcatatggtttccaacccactt | 26 |
| N501_WT2TAnew | aatcatatggtttccaacccacAa | 27 |
| N501_WT2TG | aatcatatggtttccaacccacGt | 28 |
| UTSW_N501Y_2TA | caatcatatggtttccaacccacAt | 29 |
| UTSW_N501Y_2TG | caatcatatggtttccaacccacGt | 30 |
| UTSW_N501Y_3CA | caatcatatggtttccaacccaAtt | 31 |
| UTSW_N501Y_3CG | caatcatatggtttccaacccaGtt | 32 |
| WT_2′TG | ggtagcacaccttgtaatggtgtGg | 33 |
| E484K_2′TG | ggtagcacaccttgtaatggtgtGa | 34 |
| E484K_2′TC | ggtagcacaccttgtaatggtgtCa | 35 |
| E484K_3′TA | ggtagcacaccttgtaatggtgAta | 36 |
| UTSW_E484K_3′TC | ggtagcacaccttgtaatggtgCta | 37 |
| E484K_3′TG | ggtagcacaccttgtaatggtgGta | 38 |
| FAM_UTSW_452_WT | ctaaggttggtggtaattataattaccT | 39 |
| 452_WT2′A | ctaaggttggtggtaattataattacAT | 40 |
| 452_WT2′T | ctaaggttggtggtaattataattacTT | 41 |
| 452_WT2′G | ctaaggttggtggtaattataattacGT | 42 |
| 452_WT3′A | ctaaggttggtggtaattataattaAcT | 43 |
| FAM_UTSW_452_WT3′G | ctaaggttggtggtaattataattaGcT | 18 |
| 452_WT3′T | ctaaggttggtggtaattataattaTcT | 44 |
| HEX_UTSW_L452R_Mut | ctaaggttggtggtaattataattaccG | 19 |
| L452R_2′A | ctaaggttggtggtaattataattacAG | 45 |
| L452R_2′G | ctaaggttggtggtaattataattacGG | 46 |
| L452R_2′T | ctaaggttggtggtaattataattacTG | 47 |
| L452R_3′A | ctaaggttggtggtaattataattaAcG | 48 |
| HEX_UTSW_L452R_3′G | ctaaggttggtggtaattataattaGcG | 49 |
| L452R_3′T | ctaaggttggtggtaattataattaTcG | 50 |

### Designing Primers

Provided herein are methods of designing nucleic acid primers particularly suited for identifying variants comprising any of the single nucleotide polymorphisms described herein (e.g., those encoding for N501Y, E484K, or L452R/Q substitutions in the wildtype SARS-COV-2 spike protein, SEQ ID NO: 52).

In general, methods for identifying single nucleotide polymorphisms using polymerase chain reactions (PCR) involve using a pair of primers corresponding to the wildtype and mutant alleles of the SNP in question. Each primer in the pair is labeled with a unique label and added together with a shared (common) primer that enables amplification across the site of the SNP. The presence of the WT or mutant allele can be determined by ratioing the labels and identifying the one that exceeds a given threshold. However, since the SNP corresponds to a single nucleotide difference between wildtype and mutant, these primers are often very similar, which can lead to undesired cross-binding (e.g., binding of a wildtype primer to a mutant allele or a mutant primer to a wildtype allele). To eliminate this, mutations and modifications may be introduced into the forward primers targeting the mutant or wildtype alleles that stabilize binding to the target allele and/or destabilize binding to the non-target allele.

Accordingly, in various embodiments, an improved method for designing a nucleic acid primer for detecting a single nucleotide polymorphism (SNP) in a nucleic acid is provided. In various embodiments, the method comprises: (a) providing a first nucleic acid sequence complementary to a nucleic acid comprising a mutant allele of the SNP, wherein the first nucleic acid sequence comprises a terminal 3′ nucleotide complementary to either the mutant or the wildtype nucleotide at the location of the SNP, and (b) substituting an adenine (A) for a thymine (T) (T>A substitution) one base pair upstream (5′ direction) of the terminal 3′ nucleotide complementary to the mutant or wildtype nucleotide at the location of the SNP to generate a mutant primer sequence or a wildtype primer sequence, and (c) generating a mutant primer comprising the mutant primer sequence and/or a wildtype primer comprising the wildtype primer sequence, wherein binding of the mutant primer to a wildtype allele or the binding of the wildtype primer to a mutant allele is destabilized as compared to a mutant primer or wildtype primer without the T>A substitution.

In another embodiments, a second method of generating a primer for detecting a single nucleotide polymorphism (SNP) in a nucleic acid is provided, the method comprising: (a) providing a first nucleic acid sequence complementary to a nucleic acid comprising a mutant allele of the SNP, wherein the first nucleic acid sequence comprises a terminal 3′ nucleotide complementary to the mutant nucleotide at the location of the SNP, and (b) generating a mutant primer sequence by substituting an adenine for a cytosine (C>A substitution) two base pairs upstream (5′ direction) of the terminal 3′ nucleotide that is complementary to the mutant allele, and (c) preparing an isolated nucleic acid primer (mutant primer) comprising the mutant primer sequence, wherein the mutant primer binds the mutant allele with a higher specificity than an equivalent primer without the C>A substitution.

Additional examples of methods for designing improved primers for detecting specific alleles are provided below.

The allele specific primers can be used alongside primers designed to detect the wild type allele, but not the variant allele (N501Y). These primers will be used in combination to detect either the WT or mutant variants. In various embodiments, each of the WT and mutant primers are forward primers and both use a common reverse primer designated “common.” Two common reverse primers have been generated and form different sized amplicons of either 214 nucleotides or 230 nucleotides.

The allele and wildtype specific primers can be designed according to one of two methods described herein.

In one example, a substitution is made of T>A one base pair upstream (5′ direction) of the terminal (most 3′) nucleotide that binds either the WT or mutant allele. This design is stable enough to bind either the WT or the mutant variant if present, but destabilizes the WT primer binding the mutant sequence and destabilizes the mutant primer from binding the WT sequence. In some examples herein the primer is designated with a “2′TA” at the end of the name.

In another example, a substitution is made of C>A two base pairs upstream (5′ direction) of the terminal (most 3′) nucleotide that binds either the WT or mutant allele. This can confer specificity to the mutant allele by the mutant primer. In some examples herein the primer is designated with a “3′CA” at the end of the name.

As a third example, a second version of each primer is provided made by removing the most 5′ nucleotide. This alters binding strength, which may improve the reaction. This change is designated by a “0.2” at the end of the name.

Exemplary primers designed according to these examples are provided in Table 4 below and referenced as SEQ ID NO: 9 to 13 and 24-50 herein.

TABLE 4

| Name | Sequence | SEQ ID NO: |
|---|---|---|
| Common_230_R | cagtagtgtcagcaatgtctctgcc | 9 |
| N501WT | 5′FAM-caatcatatggtttccaacccacta | 10 |
| UTSW_N501Y_3CT | 5′HEX-caatcatatggtttccaacccattt | 11 |
| UTSW_E484WT_2′TC | 5′FAM-ggtagcacaccttgtaatggtgtcg | 12 |
| UTSW_E484K | 5′HEX-cggtagcacaccttgtaatggtgtta | 13 |
| N501_WT2TA | catatggtttccaacccacaa | 24 |
| N501y2TA | catatggtttccaacccacat | 25 |
| N501_Mut | caatcatatggtttccaacccactt | 26 |
| N501_WT2TAnew | aatcatatggtttccaacccacAa | 27 |
| N501_WT2TG | aatcatatggtttccaacccacGt | 28 |
| UTSW_N501Y_2TA | caatcatatggtttccaacccacAt | 29 |
| UTSW_N501Y_2TG | caatcatatggtttccaacccacGt | 30 |
| UTSW_N501Y_3CA | caatcatatggtttccaacccaAtt | 31 |
| UTSW_N501Y_3CG | caatcatatggtttccaacccaGtt | 32 |
| WT_2′TG | ggtagcacaccttgtaatggtgtGg | 33 |
| E484K_2′TG | ggtagcacaccttgtaatggtgtGa | 34 |
| E484K_2′TC | ggtagcacaccttgtaatggtgtCa | 35 |
| E484K_3′TA | ggtagcacaccttgtaatggtgAta | 36 |
| UTSW_E484K_3′TC | ggtagcacaccttgtaatggtgCta | 37 |
| E484K_3′TG | ggtagcacaccttgtaatggtgGta | 38 |
| FAM_UTSW_452_WT | ctaaggttggtggtaattataattaccT | 39 |
| 452_WT2′A | ctaaggttggtggtaattataattacAT | 40 |
| 452_WT2′T | ctaaggttggtggtaattataattacTT | 41 |
| 452_WT2′G | ctaaggttggtggtaattataattacGT | 42 |
| 452_WT3′A | ctaaggttggtggtaattataattaAcT | 43 |
| FAM_UTSW_452_WT3′G | ctaaggttggtggtaattataattaGcT | 18 |
| 452_WT3′T | ctaaggttggtggtaattataattaTcT | 44 |
| HEX_UTSW_L452R_Mut | ctaaggttggtggtaattataattaccG | 19 |
| L452R_2′A | ctaaggttggtggtaattataattacAG | 45 |
| L452R_2′G | ctaaggttggtggtaattataattacGG | 46 |
| L452R_2′T | ctaaggttggtggtaattataattacTG | 47 |
| L452R_3′A | ctaaggttggtggtaattataattaAcG | 48 |
| HEX_UTSW_L452R_3′G | ctaaggttggtggtaattataattaGcG | 49 |
| L452R_3′T | ctaaggttggtggtaattataattaTcG | 50 |

### Modification and Detection of Nucleic Acid Primers

In various embodiments, the nucleic acid primers provided herein are used in methods (described below) to detect or identify SARS-COV-2 variants. Accordingly, the nucleic acid primers may be modified or labeled with a suitable label so that the amplified nucleic acid (amplicon) can be detected using any known method in the art (e.g., RT-qPCR, agarose gel electrophoresis, capillary electrophoresis). Suitable labels are known in the art. Some labels include radioisotypes or chemical tags. For example, radioactive phosphate can be added to the nucleic acid primer. By way of another example, the nucleic acid primers may be labeled with a fluorescent molecule (e.g., FAM or HEX). In other examples, the primers may be labeled with biotin, thiol, NHS esters, alkynes, digoxigenin, and various fluorophores including HEX, SUN, JOE, or others. This can enable separation of PCR products using capillary electrophoresis and detection using, for example, a Sanger sequencer, as described in Section III.

### II. Compositions

In various embodiments, a composition for amplifying a SARS-COV-2 nucleic acid is provided. In general, the composition comprises at least one set of nucleic acid primers suitable for amplifying a portion of the SARS-COV-2 nucleic acid comprising a target region described above (e.g., RDR1, RDR2, RDR3_4, ORF1A, ORF8, S:N501Y, S:E484K, or S:L452R/Q).

In various embodiments, the composition may comprise (i) a first primer set comprising a first nucleic acid nucleotides in length and having at least 85% sequence identity to SEQ ID NO: 1, and a second nucleic acid at least 15 nucleotides in length and having at least 85% sequence identity to SEQ ID NO: 2; (ii) a second primer set comprising a first nucleic acid at least 15 nucleotides in length and having at least 85% sequence identity to SEQ ID NO: 14, and a second nucleic acid at least 15 nucleotides in length and having at least 85% sequence identity to SEQ ID NO:15; (iii) a third primer set comprising a first nucleic acid at least 15 nucleotides in length and having at least 85% sequence identity to SEQ ID NO: 20, and a second nucleic acid at least 15 nucleotides in length and having at least 85% sequence identity to SEQ ID NO: 21; (iv) a fourth primer set comprising a first nucleic acid at least 15 nucleotides in length and having at least 85% sequence identity to SEQ ID NO: 16, and a second nucleic acid at least 15 nucleotides in length and having at least 85% sequence identity to SEQ ID NO: 17, (v) a fifth primer set comprising a first nucleic acid at least 15 nucleotides in length and having at least 85% sequence identity to SEQ ID NO: 22, and a second nucleic acid at least 15 nucleotides in length and having at least 85% sequence identity to SEQ ID NO: 23, (vi) a sixth primer set comprising a first nucleic acid at least 15 nucleotides in length and having at least 85% sequence identity to SEQ ID NO: 9, a second nucleic acid at least 15 nucleotides in length and having 85% sequence identity to SEQ ID NO: 10, and a third nucleic acid at least 15 nucleotides in length and having at least 85% sequence identity to SEQ ID NO: 11, (vii) a seventh primer set comprising a first nucleic acid at least 15 nucleotides in length and having at least 85% sequence identity to SEQ ID NO: 9, a second nucleic acid at least 15 nucleotides in length and having 85% sequence identity to SEQ ID NO: 12, and a third nucleic acid at least 15 nucleotides in length and having 85% sequence identity to SEQ ID NO: 13 and (viii) an eighth primer set comprising a first nucleic acid at least 15 nucleotides in length and having at least 85% sequence identity to SEQ ID NO: 9, a second nucleic acid at least 15 nucleotides in length and having at least 85% sequence identity to SEQ ID NO: 18, and a third nucleic acid at least 15 nucleotides in length and having 85% sequence identity to SEQ ID NO: 19.

In various embodiments, each nucleic acid in the composition is from about 15 to about 30 nucleotides, about 16 to about 30 nucleotides, about 17 to about 30 nucleotides, from about 18 to about 30 nucleotides, from about 19 to about 30 nucleotides, from about 20 to about 30 nucleotides, from about 15 to about 25 nucleotides from about 16 to about 25 nucleotides, from about 17 to about 25 nucleotides, from about 18 to about 25 nucleotides, from about 19 to about 25 nucleotides, from about 20 to about 25 nucleotides, from about 15 to about 23 nucleotides, from about 16 to about 23 nucleotides, from about 17 to about 23 nucleotides, from about 18 to about 23 nucleotides, from about 19 to about 23 nucleotides, from about 20 to about 23 nucleotides, In various aspects, each nucleic acid in the composition is independently about 15, about 16, about 17, about 18, about 19, about 20, about 21, about 22, about 23, about 24, or about 25 nucleotides in length. For example, in various embodiments, each nucleic acid in the composition is about 20 nucleotides in length.

In various embodiments, the composition comprises at least two sets, at least three sets, at least four sets, at least five sets, at least six sets, at least seven sets, or all eight sets of nucleic acids selected from (i), (ii), (iii), (iv), (v), (vi), (vii), or (viii).

In various aspects, the composition may comprise a nucleic acid primer labeled with a fluorescent label. In some aspects, the composition may comprise more than one fluorescent label. In general, any nucleic acid in the composition may be modified or labeled as described in Section I to improve annealing properties and functionality in a polymerase chain reaction.

In further embodiments, the composition may comprise a nucleic acid from a sample suspected of comprising a SARS-COV-2 virus. In some aspects, the sample is selected from a nasopharyngeal swab sample, an oropharyngeal sample, a nasal aspirate sample, a nasal or mid-turbinate swab, a nasal aspirate sample, a nasal wash sample, a throat swab sample, a bronchoalveolar lavage sample, a bronchial aspirate sample, a bronchial wash sample, an endotracheal aspirate, an endotracheal wash sample, a tracheal aspirate, a nasal secretion sample, a mucus sample, a sputum sample, a lung tissue samples, a urine sample, a saliva sample, and a fecal sample. In some aspects, the sample is obtained from a mammal. In some aspects, the sample is obtained from a human.

In various embodiments, the SARS-COV-2 virus suspected of being in the sample is a variant selected from alpha, beta, gamma, delta, epsilon, mu, iota, lambda, or omicron (e.g., BA.1, BA.1.1 and BA.2). In various embodiments, the SARS-COV-2 virus suspected of being in the sample is a variant selected from alpha, beta, gamma, iota, lambda or omicron (e.g., BA.1, BA.1.1 and BA.2).

### II. Methods

In various aspects, the nucleic acid primers described herein can be used in PCR based protocols to identify samples containing SARS CoV-2 variants. For example, a method of identifying whether a sample comprises a SARS CoV-2 variant is provided. The method can comprise amplifying nucleic acid obtained from the sample in the presence of a forward and reverse primer (selected from the forward and reverse primers described above or described below) and materials required for a polymerase chain reaction and then analyzing the resulting amplicon for fragment size differences (using electrophoresis) or copy number changes (e.g., using RT-PCR).

In various aspects, the variants identified in the methods provided herein comprise two types of mutations—(a) insertions or deletions (INDELs) or (b) single nucleotide polymorphisms (SNPs). Both types of mutations may be identified by detecting amplicons from mutant (but not wildtype) nucleic acids of a SARS-COV-2 virus in a sample. Accordingly, both methods provide for ways to distinguish mutant from wildtype amplicons. In the first, INDELS, the insertions and deletions result in an amplicon that is larger or smaller than a wildtype/native amplicon. As a result, a pair of primers spanning the region containing the INDEL can be used to detect both wildtype and mutant alleles. The resulting amplicons can be distinguished using capillary or gel electrophoresis which separate amplicons by size. In the second, SNPs, the amplicon is the same size because the mutation does not change the size of the target nucleic acid. Accordingly, here, three nucleic acid primers are typically required—one that binds selectively to the mutant allele (with one fluorescent dye: HEX), one that bind selectively to the wildtype allele (with a different fluorescent dye: FAM), and a third common reverse primer that binds to a common region, thus allowing for parallel amplification of the intervening region between the SNP and the common reverse primer. Different amplicons—labeled with the dye corresponding to the mutant or wildtype allele—can then be detected via capillary electrophoresis or other suitable methods. Methods described below provide for use of one or more of these two amplifying methods to identify a variety of SARS-COV-2 variants.

Accordingly, in various aspects, a method for detecting a SARS-COV-2 variant is provided, the method comprising: (a) amplifying a nucleic acid in a sample suspected of comprising the SARS-COV-2 variant, the variant comprising at least one insertion, deletion or single nucleotide polymorphism (SNP) as compared to a wildtype SARS-COV-2 nucleic acid, and (b) detecting an amplicon produced in (a); wherein amplifying the nucleic acid comprises: (i) performing a polymerase chain reaction using at least one set of nucleic acids (primer set), wherein the primer set amplifies a nucleic acid of the SARS-COV-2 variant comprising the insertion or deletion and the amplicon detected in (b) is larger or smaller than a corresponding amplicon generated from a wildtype SARS-COV-2 nucleic acid; and/or (ii) performing a polymerase chain reaction using a first nucleic acid primer, a second nucleic acid primer and third nucleic acid primer, wherein the first nucleic acid primer preferentially anneals to a nucleic acid comprising a mutant allele of the SNP, the second nucleic acid primer preferentially anneals to a nucleic acid comprising wildtype allele of the SNP, and the third nucleic acid allows for the amplification of a nucleic acid comprising the SNP.

In various aspects, the insertion or deletion is located in a recurrently deleted region 1 (RDR1), a recurrently deleted region 2 (RDR2), a recurrently deleted region 3_4 (RDR3_4), an open reading frame 1A (ORF1A), or an open reading frame 8 (ORF8) and/or wherein the single nucleotide polymorphism encodes an amino acid substitution in a protein of the SARS-COV-2 virus, the amino acid substitution selected from N501Y, L452R/Q, or E484K according to reference SEQ ID NO: 52.

In various aspects, the insertion or deletion is located in RDR1 and the primer set in (i) comprises a first nucleic acid at least 15 nucleotides in length and comprising at least 85% sequence homology with SEQ ID NO: 1; and a second nucleic acid at least 15 nucleotides in length and comprising at least 85% sequence homology with SEQ ID NO: 2.

In various embodiments, the insertion or deletion in RDR1 may comprise, but is not limited to, a 6 base pair deletion.

In various aspects, the insertion or deletion is in RDR2, and the primer set in (i) comprises a first nucleic acid at least 15 nucleotides in length and comprising at least 85% sequence homology with SEQ ID NO: 14; and a second nucleic acid at least 15 nucleotides in length and comprising at least 85% sequence homology with SEQ ID NO: 15. In various embodiments, the insertion or deletion in RDR2 can comprise, but is not limited to, a 3 base pair deletion, a 6 base pair deletion, a 9 base pair deletion, or a 3 base pair insertion.

In various aspects, the insertion or deletion is in RDR3_4 and the primer set in (i) comprises a first nucleic acid at least 15 nucleotides in length and comprising at least 85% sequence homology with SEQ ID NO: 20; and a second nucleic acid at least 15 nucleotides in length and comprising at least 85% sequence homology with SEQ ID NO: 21. In various embodiments, the insertion or deletion in RDR3_4 may comprise, but is not limited to, a 9 base pair deletion or a 6 base pair insertion.

In various aspects, the insertion or deletion is in ORF1A and the primer set in (i) comprises a first nucleic acid at least 15 nucleotides in length and comprising at least 85% sequence homology with SEQ ID NO: 16; and a second nucleic acid at least 15 nucleotides in length and comprising at least 85% sequence homology with SEQ ID NO: 17. In various embodiments, the insertion or deletion in ORF1A may comprise, but is not limited to, a 9 base pair deletion.

In various aspects, the insertion or deletion is in ORF8 and the primer set in (i) comprises a first nucleic acid at least 15 nucleotides in length and comprising at least 85% sequence homology with SEQ ID NO: 22; and a second nucleic acid at least 15 nucleotides in length and comprising at least 85% sequence homology with SEQ ID NO: 23. In various aspects, the insertion or deletion in ORF8 may comprise, but is not limited to, a 4 base pair insertion, a 6 base pair deletion or a 9 base pair deletion.

In any of the aspects described above, wherein an insertion or deletion is detected, the amplicon produced in (b) may be larger or smaller as compared to a corresponding amplicon produced by amplifying a wildtype SARS-COV-2 nucleic acid in the presence of the at least one primer pair and wherein the method further comprises identifying the SARS-CO-V-2 variant by detecting the larger or smaller amplicon as compared to a control sample comprising a wildtype SARS-COV-2 nucleic acid.

In further aspects, the method comprises detecting a SARS-COV-2 variant by detecting a single nucleotide polymorphism (SNP).

In various aspects, the single nucleotide polymorphism results in a N501Y amino acid substitution in the encoded protein, and the primer set comprises a first nucleic acid at least 15 nucleotides in length and comprising at least 85% sequence homology with SEQ ID NO: 9; and a second nucleic acid at least 15 nucleotides in length and comprising at least 85% sequence homology with SEQ ID NO: 10, and a third nucleic acid at least 15 nucleotides in length and comprising at least 85% sequence homology with SEQ ID NO 11.

In further aspects, the single nucleotide polymorphism results in a E484K amino acid substitution in the encoded protein, and the primer set comprises a first nucleic acid at least 15 nucleotides in length and having at least 85% sequence identity to SEQ ID NO: 9, a second nucleic acid at least 15 nucleotides in length and having 85% sequence identity to SEQ ID NO: 12, and a third nucleic acid at least 15 nucleotides in length and having 85% sequence identity to SEQ ID NO: 13.

In another aspect, the single nucleotide polymorphism results in a L452R/Q amino acid substitution in the encoded protein, and the primer set comprises a first nucleic acid at least 15 nucleotides in length and having at least 85% sequence identity to SEQ ID NO: 9, a second nucleic acid at least 15 nucleotides in length and having at least 85% sequence identity to SEQ ID NO: 18, and a third nucleic acid at least 15 nucleotides in length and having 85% sequence identity to SEQ ID NO: 19.

In any of the methods described herein, the nucleic acids in each primer set may each independently have a length of about 15 to about 30 nucleotides, about 16 to about 30 nucleotides, about 17 to about 30 nucleotides, from about 18 to about 30 nucleotides, from about 19 to about 30 nucleotides, from about 20 to about 30 nucleotides, from about 15 to about 25 nucleotides from about 16 to about 25 nucleotides, from about 17 to about 25 nucleotides, from about 18 to about 25 nucleotides, from about 19 to about 25 nucleotides, from about 20 to about 25 nucleotides, from about 15 to about 23 nucleotides, from about 16 to about 23 nucleotides, from about 17 to about 23 nucleotides, from about 18 to about 23 nucleotides, from about 19 to about 23 nucleotides, from about 20 to about 23 nucleotides, In various aspects, each nucleic acid in each primer set used in the methods herein is independently about 15, about 16, about 17, about 18, about 19, about 20, about 21, about 22, about 23, about 24, or about 25 nucleotides in length. For example, in various embodiments, each nucleic acid in each primer set is about 20 nucleotides in length.

In any of these methods of detecting a SNP, the first nucleic acid primer may be labeled with a first fluorescent marker, the second nucleic acid may be labeled with a second fluorescent marker, wherein the first and second fluorescent marker are independently detectable in the same sample, and wherein the method further comprises identifying the SARS-COV-2 variant when a ratio of the first fluorescent marker to the second fluorescent marker exceeds a threshold.

In any of the methods provided herein, more than one set of nucleic acid primers may be used to detect more than one mutation or allele in the same sample. In other words, the methods described herein may be multiplexed to generate a mutation map of a single sample, which may then be used to identify the SARS-COV-2 variant.

Accordingly, in various aspects, the methods provided herein may comprise amplifying one or more the nucleic acids in a sample suspected of comprising the SARS-COV-2 variant using at least one set, at least two sets, at least three sets, at least four sets, at least five sets, at least six sets, at least seven sets, or at least eight sets of nucleic acid primers (primer sets) selected from:
(i) a first primer set to detect an insertion or deletion in RDR1 comprising a first nucleic acid at least 15 nucleotides in length and having at least 85% sequence identity to SEQ ID NO: 1, and a second nucleic acid at least 15 nucleotides in length and having at least 85% sequence identity to SEQ ID NO: 2,
(ii) a second primer set to detect an insertion or deletion in RDR2 comprising a first nucleic acid at least 15 nucleotides in length and having at least 85% sequence identity to SEQ ID NO: 14, and a second nucleic acid at least 15 nucleotides in length and having at least 85% sequence identity to SEQ ID NO:15,
(iii) a third primer set to detect an insertion or deletion in RDR3_4 comprising a first nucleic acid at least 15 nucleotides in length and having at least 85% sequence identity to SEQ ID NO: 20, and a second nucleic acid at least 15 nucleotides in length and having at least 85% sequence identity to SEQ ID NO: 21,
(iv) a fourth primer set to detect an insertion or deletion in ORF1 a comprising a first nucleic acid at least 15 nucleotides in length and having at least 85% sequence identity to SEQ ID NO: 16, and a second nucleic acid at least 15 nucleotides in length and having at least 85% sequence identity to SEQ ID NO: 17,
(v) a fifth primer set to detect an insertion or deletion in ORF8 comprising a first nucleic acid at least 15 nucleotides in length and having at least 85% sequence identity to SEQ ID NO: 22, and a second nucleic acid at least 15 nucleotides in length and having at least 85% sequence identity to SEQ ID NO: 23,
(vi) a sixth primer set to detect an N501Y SNP comprising a first nucleic acid at least 15 nucleotides in length and having at least 85% sequence identity to SEQ ID NO: 9, a second nucleic acid at least 15 nucleotides in length and having 85% sequence identity to SEQ ID NO: 10, and a third nucleic acid at least 15 nucleotides in length and having at least 85% sequence identity to SEQ ID NO: 11,
(vii) a seventh primer set to detect an E484K SNP comprising a first nucleic acid cat least 15 nucleotides and having at least 85% sequence identity to SEQ ID NO: 9, a second nucleic acid at least 15 nucleotides in length and having 85% sequence identity to SEQ ID NO: 12, and a third nucleic acid at least 15 nucleotides in length and having 85% sequence identity to SEQ ID NO: 13 and
(viii) an eighth primer set to detect a L452R/Q SNP comprising a first nucleic acid at least 15 nucleotides in length and having at least 85% sequence identity to SEQ ID NO: 9, a second nucleic acid at least 15 nucleotides in length and having at least 85% sequence identity to SEQ ID NO: 18, and a third nucleic acid at least 15 nucleotides in length and having 85% sequence identity to SEQ ID NO: 19; and wherein the SARS-COV-2 variant is identified based on the presence or absence of the insertion or deletion in RDR1, RDR2, RDR3_4, ORF1a, ORF8, or the presence or absence of the N501Y, NE484K, or L452R/Q SNPs.

In various embodiments, the nucleic acid can be amplified using any standard polymerase chain reaction known in the art. In various embodiments, the polymerase chain reaction comprises mixing the nucleic acid with the forward and reverse primers, a polymerase, and sufficient isolated nucleotides (dNTPs or ddNTPs if chain termination is desired) and then cycling the mixture in a thermocycler through a series of stages at different temperatures. An exemplary thermocycler protocol is described in Table 5 below, but modifications may be made according to methods in the art to account for variables such as the melting temperature of the primers and the specific allele that is targeted.

TABLE 5
Exemplary Thermocycler Protocol

| Temperature | Time | | |
|---|---|---|---|
| 25° C. | 2 | minutes | |
| 50° C. | 15 | minutes | |
| 95° C. | 2 | minutes | |
| 95° C. | 10 | seconds | Repeat 45 cycles |
| 61° C. | 30 | seconds | |
| 72° C. | 30 | seconds | |
| 72° C. | 5 | minutes | |

| | |
|---|---|
| 4° C. | hold |

In various embodiments, the primers described above allow for specific fragment sizes to be enriched in a sample containing a variant as compared to a wildtype virus. For example, as described in Table 6 below, amplicons from variants having an S69_70 deletion can comprise a 182 bp amplicon (relative to a 188 bp amplicon in WT). As an additional example, amplicons from variants having an S144 deletion can comprise a 153 bp amplicon (relative to a 156 bp amplicon for WT). As another example, amplicons from variants having an ORFA1A deletion can comprise a 122 bp amplicon (relative to a 132 bp amplicon for WT). These differences in fragment sizes can be readily detected by a Sanger sequencer when separated by capillary electrophoresis.

TABLE 6

| | | Base | WT | Mutant |
|---|---|---|---|---|
| | | pairs | amplicon | amplicon |
| Target | Mutation | deleted | size | size |
| S69/70 | 21765_21770del | 6 bp | 188 bp | 182 bp |
| S144 | 21991_21993del | 3 bp | 156 bp | 153 bp |
| ORFA1A | 11288_11296del | 9 bp | 132 bp | 122 bp |
| N1 | N/A | N/A | 68 bp | N/A |

Table 7 below provides a summary of the expected mutations/insertions/deletions or SNPs at each of the targeted regions in many known SARS-COV-2 variants. The list is exemplary and should not be considered limiting. Additional alleles not listed could also be envisioned and may be identified by one of ordinary skill using the nucleic acid primers, compositions and methods provided herein. For example, as described in more detail below, unexpected/unpredicted mutations at a SNP may be identified when both the mutant and wildtype primer fail to produce a sufficient amplicon (under proper controls). This is considered a “fall off” situation and, as shown in the Table 7 below, can occur in some variants.

TABLE 7

| Variant | RDR1 | RDR2 | RDR3_4 | ORF1A | ORF8 | N501 | E484 | L452 |
|---|---|---|---|---|---|---|---|---|
| WT | — | — | — | — | — | — | — | — |
| Alpha | 6 bp | 3 bp | | 9 bp | | N501Y | WT | WT |
| | del | del | | del | | | | |
| Beta | | | 9 bp del | 9 bp | | N501Y | E484K | |
| | | | (RDR4) | del | | | | |
| Gamma | | | | 9 bp | 4 bp ins | N501Y | E484K | |
| | | | | del | | | | |
| Iota | | | | 9 bp | | | E484K | |
| | | | | del | | | | |
| Lambda | | | 21 bp del | 9 bp | | | | L452Q |
| | | | (RDR3) | del | | | | (fall off), |
| | | | | | | | | reduced |
| | | | | | | | | amplification |
| | | | | | | | | at mutant |
| | | | | | | | | allele |
| Delta | | 6 bp | | | 6 bp | | Reduced WT | L452R |
| | | del | | | del | | amplification, | |
| | | | | | | | without | |
| | | | | | | | increase in | |
| | | | | | | | mutant allele | |
| | | | | | | | amplification | |
| Mu | | 3 bp ins. | | | | N501Y | E484K | |
| Epsilon | | | | | | | E484K | |
| Omicron | 6 bp | 9 bp | 6 bp ins. | 9 bp | | N501Y | | |
| | del | del | | del | | | | |

Table 8 provides more detailed information about the amplicons expected to be produced using the exemplary primers provided herein. For regions where an insertion/deletion is expected (RDR1, RDR2, RDR3_4, ORF1A or ORF8) expected amplicon size for each variant is provided. For regions where a SNP is expected (N501Y, E484K, L452R) the type of amplicon expected (e.g., mutant or wildtype or none) is provided. As noted, if the variant has a substitution at or near the SNP not accounted for in the primer design, the mutant and/or wildtype allele may have a reduced ability to amplify—which can be interpreted as a mutation alongside the others (e.g., Lambda has a L452Q polymorphism which causes a “fall off” of the expected signal expected from a primer targeting a mutant allele (L452R)).

TABLE 8

| | RDR1 | RDR2 | RDR3_4 | ORF1A | ORF8 | N501* | E484K* | L452R* |
|---|---|---|---|---|---|---|---|---|
| | SEQ ID | SEQ ID | SEQ ID | SEQ ID | SEQ ID | SEQ ID | SEQ ID | SEQ ID |
| | NO: 1 | NO: 14 | NO: 20 | NO: 16 | NO: 22 | NO: 10 | NO: 12 | NO: 18 |
| Variant | and 2 | and 15 | and 21 | and 17 | and 23 | and 11 | and 13 | and 19 |
| WT | 188 bp | 156 bp | 282 bp | 132 bp | 226 bp | WT | WT | WT |
| Alpha | 182 bp | 153 bp | 282 bp | 122 bp | 226 bp | MUT | WT | WT |
| Beta | 188 bp | 156 bp | 272 bp | 122 bp | 226 bp | MUT | MUT | WT |
| Gamma | 188 bp | 156 bp | 282 bp | 122 bp | 230 bp | MUT | MUT | WT |
| Iota | 188 bp | 156 bp | 282 bp | 122 bp | 226 bp | WT | MUT | WT |
| Lambda | 188 bp | 156 bp | 261 bp | 122 bp | 226 bp | WT | WT | MUT**(reduced |
| | | | | | | | | mutant |
| | | | | | | | | allele) |
| Delta | 188 bp | 156 bp | 282 bp | 132 bp | 219 bp | WT | WT | MUT***(reduced |
| | | | | | | | | WT |
| | | | | | | | | allele) |
| Mu | 188 bp | 159 bp | 282 bp | 132 bp | 226 bp | MUT | MUT | WT |
| Epsilon | 188 bp | 156 bp | 282 bp | 132 bp | 226 bp | WT | WT | MUT |
| Omicron | 182 bp | 147 bp | 288 bp | 122 bp | 217 bp | MUT | WT | WT |

*Location of substitution caused by single nucleotide polymorphism, according to SEQ ID NO: 52
**Mutant allele is identified as a reduced amplification of the mutant allele (L452R), without an expected increase in amplification in wildtype allele.
**Mutant allele is identified has having a reduced amplification of the wildtype allele without an expected increase in mutant (L452R) allele.

Accordingly, in various embodiments, the amplicon generated from the amplifying reaction is analyzed using capillary electrophoresis and a Sanger sequencer. In other embodiments, the amplicon may be analyzed using gel electrophoresis. In various embodiments, the primers (and thus, fragments) are further labeled with a fluorescent marker (e.g, FAM or HEX) which facilitates their detection in a Sanger sequencer.

In another embodiment, a method for identifying whether a sample contains an N501Y, E484K, or L452R variant of SARS-CoV-2 is provided. The method can comprise amplifying nucleic acid from the sample in the presence of primers designed to be selective for a wildtype or a mutant (N501Y, E484K, or L452R) allele and then detecting a “wildtype” or a “mutant” amplicon. For example, a wildtype amplicon can comprise an amplicon generated when “wildtype” primers are used (or that is detected by the “wild-type” fluorescent marker). A mutant amplicon can comprise an amplicon generated when “mutant” primers are used (or that is detected by the “mutant” fluorescent marker). Exemplary primers for this method are described in Table 9 below.

TABLE 9

| | | SEQ |
|---|---|---|
| | | ID |
| Name | Sequence | NO: |
| Common_230_R | cagtagtgtcagcaatgtctctgcc | 9 |
| N501WT | -caatcatatggtttccaacccacta | 10 |
| UTSW_N501Y_3CT | caatcatatggtttccaacccattt | 11 |
| UTSW_E484WT_ | ggtagcacaccttgtaatggtgtcg | 12 |
| 2′TC | | |
| UTSW_E484K | cggtagcacaccttgtaatggtgtta | 13 |
| UTSW_L452WT_ | ctaaggttggtggtaattataattagct | 18 |
| 3′G | | |
| UTSW_L452R | ctaaggttggtggtaattataattaccg | 19 |

As described for the other variants, these primers can be labeled with fluorescent markers (e.g., FAM or HEX) to facilitate detection with a Sanger sequencer or RT-PCR detector. In various embodiments, more than one fluorescent marker is used in an experiment (e.g., mutant=HEX and wild-type=FAM) to more clearly distinguish mutant or wildtype amplicons.

In various embodiments, a sample is identified has having a SARS-CoV-2 variant if a shortened fragment (relative to wildtype) is detected, or if a replicon is identified or enriched when amplified using mutant allele specific primer.

In various embodiments, the sample containing SARS-CoV-2 comprises a sample obtained from an individual suspected of being infected with SARS-CoV-2. For example, the sample can be selected from a nasopharyngeal swab sample, an oropharyngeal sample, a nasal aspirate sample, a nasal or mid-turbinate swab, a nasal aspirate sample, a nasal wash sample, a throat swab sample, a bronchoalveolar lavage sample, a bronchial aspirate sample, a bronchial wash sample, an endotracheal aspirate, an endotracheal wash sample, a tracheal aspirate, a nasal secretion sample, a mucus sample, a sputum sample, a lung tissue samples, a urine sample, a saliva sample, and a fecal sample or other sample obtained from the subject.

In various embodiments, the subject is an animal. The animal may be a mammal. The mammal may be a human, pet, livestock, wildlife, or other animal such as those found in zoos. In some embodiments, the subject is a pet such as of a dog (canine), a cat (feline), a ferret, hamster, a rabbit, or other pet. In some embodiments, the subject is livestock such as a horse (equine), cattle (bovine), a llama, a buffalo, a goat (hircine), a sheep (caprine), a pig (porcine), or other livestock. In some embodiments, the subject is an animal typically found in a zoo such as a tiger, lion, leopard, zebra, giraffe, antelope, elephant, hippopotamus, hyena, non-human primates (lemurs, monkeys, apes, etc.), bats, mink or other animal found in a zoo. In some embodiments, the animal is considered wildlife, such as a deer, elk, moose, raccoon, bat, fox, wild cat, shrew, vole, squirrel, or other wild animal. In various embodiments the subject is human.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Introduction to Examples

New strains of SARS-CoV-2 have emerged with clinically significant genetic mutations. These variants impact transmission, vaccine response, mortality, and drug resistance (monoclonal antibodies). Therefore, methods for real-time identification of SARS-CoV-2 variant strains are of exigent need in both clinical and public health arenas. Whole-genome sequencing (WGS) is the current gold standard for SARS-CoV-2 variant identification; however, broad WGS adoption is hampered by financial restrictions and requirements for specialized equipment and bioinformatics expertise. While these limitations may be mitigated through centralized high-volume testing, the consequent increased turnaround time for batching, data deconvolution, and sequence analysis exceeds what is clinically actionable for most patient assessments. A simple, on-site reflex test of positive specimens for variants is a practical solution.

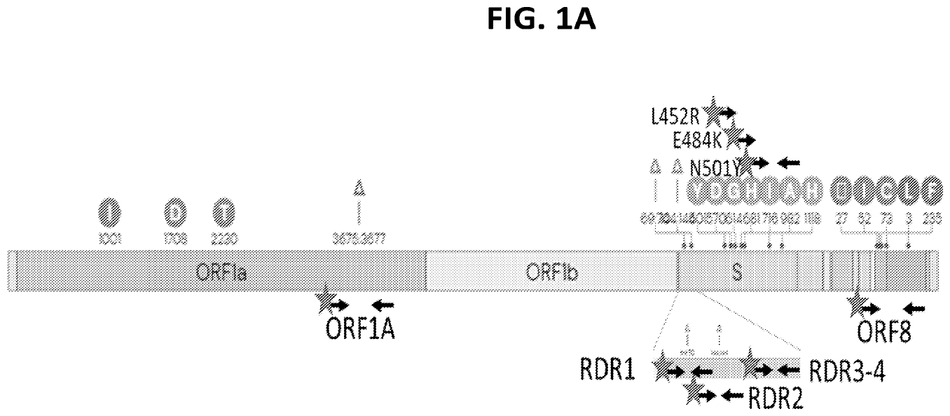

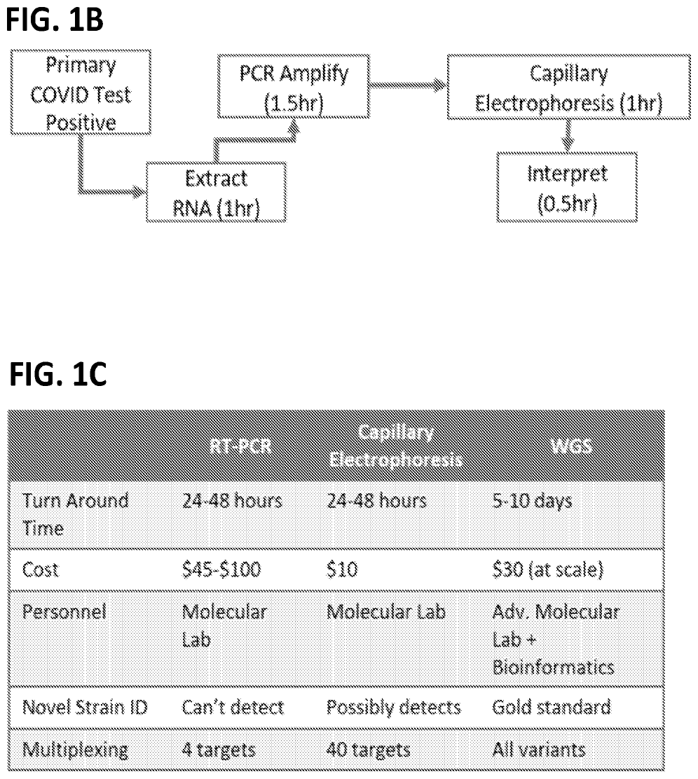

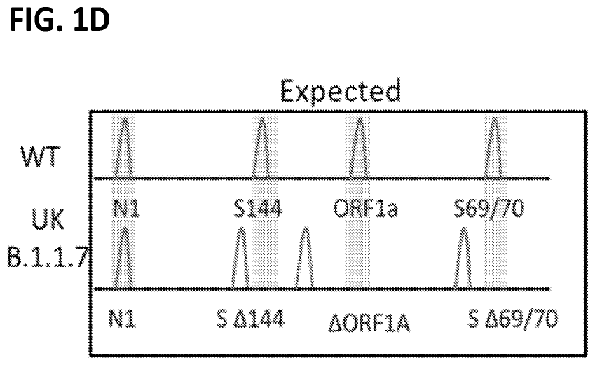

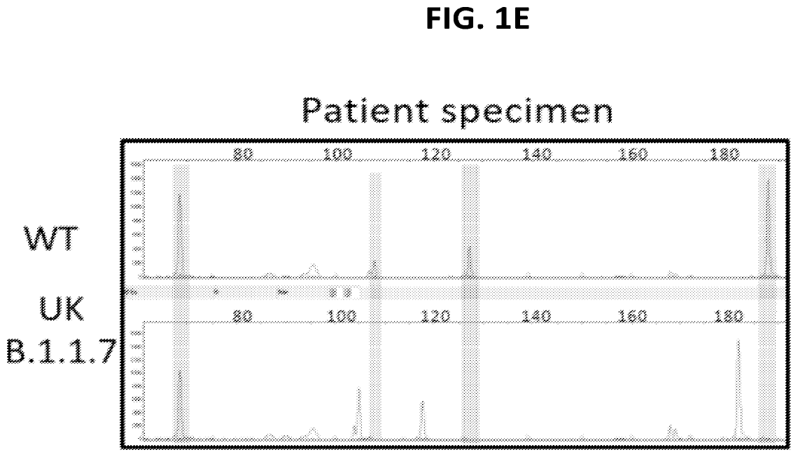

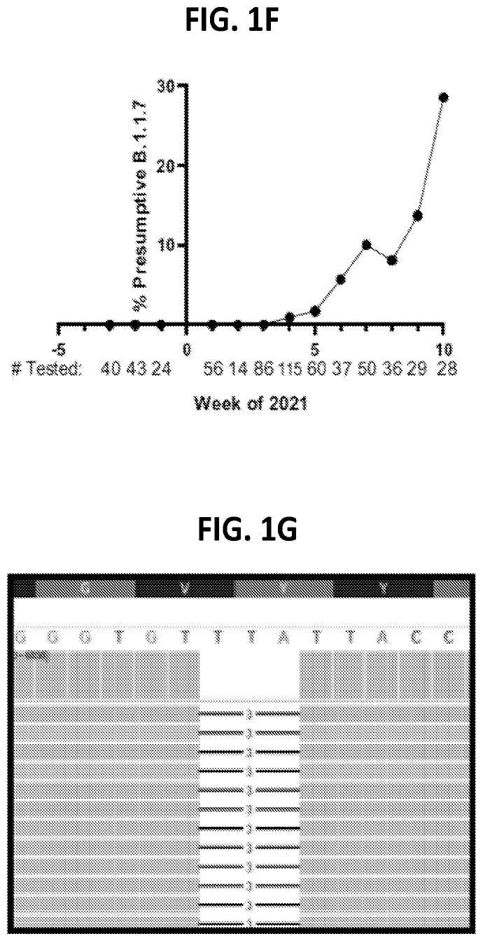

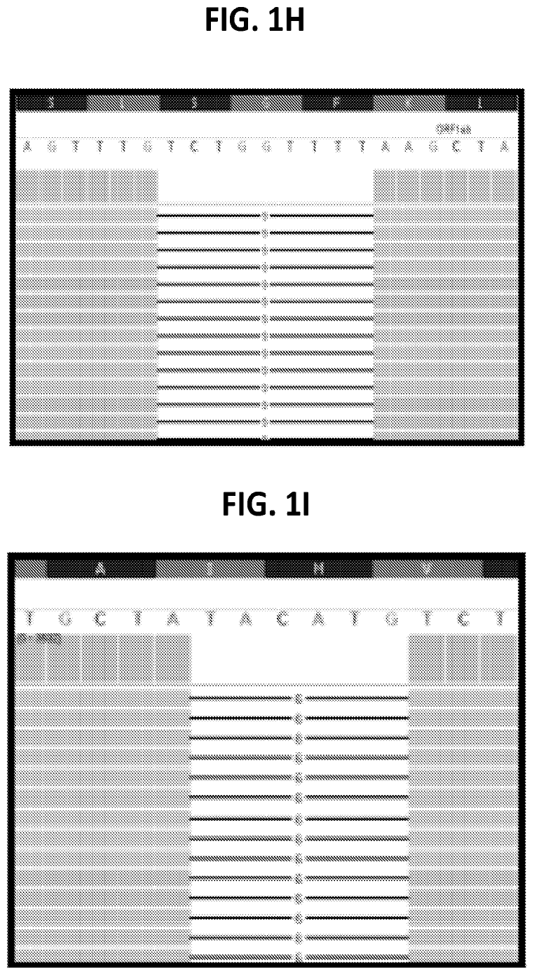

Here, a rapid, cost-effective, and scalable method utilizing fragment analysis by capillary electrophoresis is described to detect known SARS-CoV-2 variants in a routine clinical setting. In this procedure, extracted RNA is amplified to create fluorescently marked amplicons that are separated by capillary electrophoresis on a Sanger sequencer. Variants are then identified by their abnormal amplicon sizes. By measuring the resultant changes in fragment length, this fragment analysis assay permits a broader range of variant detection compared to primer-probe based RT-qPCR. The reagent and instrumentation requirements for this assay are modest: fluorescently labeled primers, a thermocycler, and Sanger sequencer. Moreover, the time requirement (4 hours, FIG. 1B), simplicity, and use of 96-well format support the assay's scalability. Amplicons can be designed up to 500 bp in length with up to four fluorescent labels, making the assay adaptable to detect other SARS-CoV-2 variants as they are identified (FIG. 1C, potential to multiplex 20-40 targets). This assay provides a mechanism for routine clinical laboratories to screen positive specimens for SARS-CoV-2 variants of concern (VOC) that can be easily and broadly implemented with limited financial burden or bioinformatics requirements.

### Materials and Methods

The following materials, primers and protocols were used in the Examples described herein.

### Materials:

Applied Biosystems Instrument 3730XL
Thermo TaqPath One Step RT-qPCR kit (containing Master Mix: TaqPath polymerase (4×) and nuclease free water).

### Primers.

N1: Forward Primer-FAM (100 μM), Reverse primer (100 μM)
RDR1: Forward Primer-FAM (100 μM), Reverse primer (100 μM)
RDR2: Forward Primer-FAM (100 μM), Reverse primer (100 μM)
ORF1A: Forward Primer-FAM (100 μM), Reverse primer (100 μM)

Primers were ordered desalted and dried with no label on reverse primer and FAM on forward primer only. Primer sequences are provided in Table 10 below.

TABLE 10

| | | SEQ ID |
|---|---|---|
| Name | Sequence | NO: |
| S69/70_F: | 5′-FAM-CGTGGTGTTTATTACCCTGACAAAG-3′ | 1 |
| S69/70_R: | 5′-TCAGTGGAAGCAAAATAAACACCAT-3′ | 2 |
| S144_F: | 5′-FAM-ACGCTACTAATGTTGTTATTAAAGTCT-3′ | 3 |
| S144_R: | 5′-TCTGAACTCACTTTCCATCCAACT-3′ | 4 |
| ORF1A_F: | 5′-FAM-TGCCTGCTAGTTGGGTGATG-3′ | 5 |
| ORF1A_R: | 5′-TGCTGTCATAAGGATTAGTAACACT-3′ | 6 |
| N1_F: | 5′-FAM-GACCCCAAAATCAGCGAAAT-3′ | 7 |
| N1_R: | 5′-TCTGGTTACTGCCAGTTGAATCTG-3′ | 8 |
| UTSW_Common_230_R | 5′-CAGTAGTGTCAGCAATGTCTCTGCC | 9 |
| UTSW_N501WT | 5′-FAM-CAATCATATGGTTTCCAACCCACTA | 10 |
| UTSW_N501Y_3′T | 5′-HEX-CAATCATATGGTTTCCAACCCATTT | 11 |
| UTSW_E484WT_3′G | 5′-FAM-GGTAGCACACCTTGTAATGGTGTCG | 12 |
| UTSW_E484K | 5′-HEX-CGGTAGCACACCTTGTAATGGTGTTA | 13 |
| Yale_144del_F: (RDR_F) | 5′-FAM-GAATTTGAATGATCCATTTTTGGGTG | 14 |
| UTSW_RDR2_R: | 5′-GGCTGAGAGACATATTCAAAGTGC | 15 |
| UTSW_ORF1A_F + 1: | 5′-FAM-TGCCTGCTAGTTGGGTGATG | 16 |
| UTSW_ORF1A_R + 5: | 5′-GTTCTTGCTGTCATAAGGATTAGTAACAC | 17 |
| UTSW_L452WT_3′G | 5′-FAM-CTAAGGTTGGTGGTAATTATAATTAGCT | 18 |
| UTSW_L452R | 5′-HEX-CTAAGGTTGGTGGTAATTATAATTACCG | 19 |
| UTSW_RDR3-4_F | 5′-FAM-GGACCTTGAAGGAAAACAGGG | 20 |
| UTSW_RDR3-4_R | 5′-TGAAGATAACCCACATAATAAGCTGC | 21 |
| UTSW_ORF8_F | 5′-FAM-GCCAGAAACCTAAATTGGGTAGT | 22 |
| UTSW_ORF8_R | 5′-CCCACTGCGTTCTCCATTCTG | 23 |

Controls: Previous positive SARS-CoV-2 specimens. B.1.1.7 Control material: Twist Synthetic SARS-CoV-2 RNA control: Control 14 (B.1.1.7 variant: England/205041766/2020). Ladder: GS500-LIZ.

### Protocol:

### Method 1 COVID-Variant Fragment Analysis:

Thaw all items on ice. Vortex and centrifuge all items. Create (10×) PCR primer mix: Combine primers as described below in Table 11 (sequences of primers are found in Table 10).

TABLE 11
10X PCR Primer Mix

| Component | Quantity | | Quantity | |
|---|---|---|---|---|
| RDR1_F (Thermo_S-F) Fwd Primer (100 uM stock) | 5 | μl | 2 | μl |
| RDR1_R (Thermo_S-R) Rev primer (100 uM stock) | 5 | μl | 2 | μl |
| UTSW_ORF1A_F + 1 Forward Primer (100 uM stock) | 2.5 | μl | 1 | μl |
| UTSW_ORF1A_R + 5 Reverse primer (100 uM stock) | 2.5 | μl | 1 | μl |
| UTSW_ORF8_F Forward Primer (100 uM stock) | 10 | μl | 4 | μl |
| UTSW_ORF8_R Reverse primer (100 uM stock) | 10 | μl | 4 | μl |
| UTSW_RDR3-4_F Forward Primer (100 uM stock) | 7 | μl | 2.8 | μl |
| UTSW_RDR3-4_R Reverse primer (100 uM stock) | 7 | μl | 2.8 | μl |
| Yale_144del_F Forward Primer (100 uM stock) | 2.5 | μl | 1 | μl |
| UTSW_RDR2_R Reverse primer (100 uM stock) | 2.5 | μl | 1 | μl |
| UTSW_N501WT Forward Primer (100 uM stock) | 10 | μl | 4 | μl |
| UTSW_N501Y_3′T Forward Primer (100 uM stock) | 10 | μl | 4 | μl |
| UTSW_E484WT_2′C Forward Primer (100 uM stock) | 10 | μl | 4 | μl |
| UTSW_E484K_3′G Forward Primer (100 uM stock) | 10 | μl | 4 | μl |
| UTSW_L452WT_3′G Forward Primer (100 uM stock) | 5 | μl | 2 | μl |
| UTSW_L452R_Mut Forward Primer (100 uM stock) | 5 | μl | 2 | μl |
| UTSW_Common_230_R Reverse primer (100 uM stock) | 25 | μl | 10 | μl |
| Nuclease Free Water | 371 | μl | 148.4 | μl |
| Total | 500 | μl | 200 | μl |

### Real Time Quantitative Polymerase Chain Reaction Set Up.

Prepare Master Mix according to Table 12 below. Scale up as necessary and include 10% extra for pipetting errors. Combine the first 3 components first to be the Master Mix. Dispense 5 μL of Master Mix to each well, then add the samples to each well individually changing tips between samples. Note: TaqPath polymerase is viscous and should be drawn up slowly. Seal the plate with clear AMP*** film, vortex and centrifuge at 2000 rpm for 1 minute to collect the contents at the bottom and reduce bubbles.

TABLE 12
TaqPath RT-qPCR Master Mix

| | | | |
|---|---|---|---|
| | Quantity | Quantity | Quantity |
| Component | (1rxn) | (50rxn) | (96rxn) |

| | | | | | | |
|---|---|---|---|---|---|---|
| TaqPath polymerase (4x) | 2.5 | μL | 137.5 | μL | 264 | μL |
| 10X PCR primer mix | 1 | μL | 55 | μL | 106 | μL |
| Nuclease Free Water | 1.5 | μL | 82.5 | μL | 158 | μL |
| Sample RNA | 5 | μL | 275 | μL | 528 | μL |
| Total | 10 | μL | 550 | μL | 1,056 | μL |

### PCR Amplification.

Thermocyler settings are modified from the CDC SARS-CoV-2 assay for TaqPath polymerase to include a 30 second extension step at 72° C. for the amplicons larger than traditional qPCR amplicons. Set the thermocycler to the program listed in Table 13 below. After completion, store the plate on ice protected from light, or in a 4° C. refrigerator until electrophoresis can be completed. Store at −20° C. for longer term storage.

TABLE 13
Thermocycler Settings for PCR Reaction

| Temperature | Time | | |
|---|---|---|---|
| 25° C. | 2 | minutes | |
| 50° C. | 15 | minutes | |
| 95° C. | 2 | minutes | |
| 95° C. | 10 | seconds | Repeat 45 cycles |
| 61° C. | 30 | seconds | |
| 72° C. | 30 | seconds | |
| 72° C. | 5 | minutes | |

| | |
|---|---|
| 4° C. | hold |

Preparation for electrophoresis. Add 8.5 μL of Hi-Di Formamide+0.5 μL of LIS size standard to each well of a new plate. Add 1 μL of PCR amplified specimen from step 5 to each well of the same plate. Seal the plate with a clear plastic film. Vortex for 2-5 seconds. Centrifuge at 2000 rpm for 1 minute. Denature fragments by heating plate for 3 minutes at 95° C. Return to ice (protected from light) until the sample is run by pelectrophoresis.

Capillary electrophoresis. Confirm the instrument is calibrated properly for LIS and FAM dyes prior to running the sample by using the DS-33 Matrix standard kit. Remove clear plastic adhesive film with the 96-well plate septum. Plate the plate into the instrument and close the lid. Select or create the settings that are appropriate to the run modality according to capillary length, polymer type and number of capillaries.

Interpretation: The expected wild type and variant fragments are described in Table 14-15 and FIG. 1 (Example 1). If no N1 nor any other variants are present, then the result is “SARS-CoV-2 not detected.” Absence of a fragment is not definitive evidence of a variant unless the other fragments are present at high levels. Different variants described in the literature will have different combinations of the variants listed in Table 14 and FIG. 1.

TABLE 14
Expected WT and mutant amplicon size

| | | Base | WT | Mutant |
|---|---|---|---|---|
| | | pairs | amplicon | amplicon |
| Target | Mutation | deleted | size | size |
| S69/70 | 21765_21770del | 6 bp | 188 bp | 182 bp |
| S144 | 21991_21993del | 3 bp | 107 bp | 104 bp |
| ORFA1A | 11288_11296del | 9 bp | 127 bp | 118 bp |
| N1 | N/A | N/A | 68 bp | N/A |

TABLE 15
Recurrent Artifacts

| Location | Comments | Location | Comments |
|---|---|---|---|
| <50 | Primer dimers | 162 bp | Broad |
| 88 | Broad | 176, 177, | Recurrent, |
| | | 182 | non-specific |
| 91 | Broad | 200, 205 | Recurrent, |
| | | | non-specific |
| 97 | Broad | | |

Next Generation Sequencing. First-strand cDNA was synthesized from samples using the PrimeScript™ IV 1st strand cDNA Synthesis (5 μl of RNA). cDNA was converted into an NGS library with the Swift SNAP SARS-CoV-2 Kit (Integrated DNA Technologies) using tiled primer pairs in a single tube to target the 29.9 kb viral genome and sequenced on the Illumina® MiSeq® System at 2×250 bp. For the Ct value from 4 to 15, make the 1:2 dilution for cDNA before the library preparation. For the Ct value within 29, follow the protocol in section “Prepare SNAP Libraries”. Primers were designed against the NCBI Reference Sequence NC_045512.2 (Severe acute respiratory syndrome coronavirus 2 isolate Wuhan-Hu-1, complete genome, SEQ ID NO: 51). Pooling: Following the Normalase I incubation, 5 μl of 49 individual libraries were pooled into a 1.5 mL screw cap microfuge tube (total volume is 245 μl); add 49 μl of Normalase II Master Mix was added to the prepared pool and incubated for 15 min at 37° C. Then 9.8 μl of Reagent X1 was added into the prepared pool and incubated for 2 min at 95° C. The final multiplexed library pool was then of equal molarity and ready for sequencing (final concentration=˜4 nM)

Bioinformatic Analysis. Raw sequences were trimmed for quality and adapter sequence using TrimGalore (Github) with a quality threshold of 25. Trimmed reads were aligned with minimap2 to the SARS-COV-2 reference genome (NC_045512, SEQ ID NO: 51). Primer sequences were removed using primerclip (Github). Alignments were sorted and indexed using samtools. Duplicate reads were removed using picard MarkDuplicates (Github). Variants were detected using BCFtools and VarScan. Consensus sequences were determined using BCFtools. Variants were annotated using snpEFF. Strain lineage was determined using PANGOLIN.

### Example 1

For proof of concept, we targeted three deletion mutations initially described in the UK B.1.1.7 VOC (later designated Alpha). The B.1.1.7 strain was originally discovered due to S-gene target failure (SGTF) secondary to the Spike gene HV69_70del variant in a SARS-CoV-2 commercial assay. While present in B.1.1.7, the HV69_70del is not specific to this strain (55% of northeast U.S. HV69_70del mutants are not B.1.1.7); therefore, the S-gene F144 deletion mutation, characteristic of B.1.1.7, was included to improve specificity. Frequent in-frame deletions near the 69/70 and 144 amino acid positions suggest this is a mutational hotspot, which could arise in other emerging strains. The third deletion in ORF1A (SDF3675_3677del) is present in B.1.1.7 (UK variant, alpha), B.1.351 (South African, originally N150Y.2, later designated Beta), and P.1 (Brazil, later designated Gamma). Therefore, detecting an ORF1A deletion without the S gene deletions is suggestive of the B.1.351 or P.1 variants (e.g., Beta or Gamma). (FIG. 1A, 2G). Lastly, the CDC N1 primer pair was added as an internal positive control and could allow the screen to be adapted to routine or confirmatory testing within the limits of detection.

We validated this method focusing on the principle SARS-CoV-2 VOC impacting European countries and the United States: B.1.1.7 (alpha). An initial screen of 182 retrospective (12/1/20-1/20/21) and 466 prospective SARS-CoV-2 positive nasopharyngeal specimens (1/25/21-3/11/21) was performed. No variants were identified among retrospective specimens (0/182). On the third week in January 2021, the first isolate was identified with characteristic shortened amplicons reflecting B.1.1.7/alpha deletions (FIG. 1D, 1E, Interpretive criteria described above in Methods). This deletion pattern was confirmed in control RNA containing the B.1.1.7 (alpha) sequence. The suspected B.1.1.7 (alpha) isolate and positive control sample were sequenced by WGS. Characteristic variants were confirmed (all in isolate and 16/17 in control, FIG. 1G-1). Furthermore, these samples were classified as B.1.1.7 (alpha) using PANGOLIN.

Overall, we detected 32 specimens consistent with B.1.1.7 (n=23 patient specimens, n=9 control RNA, 12 WGS verified/11 pending sequencing). All of the specimens had low SARS-CoV-2 RT-PCR CT values, and each displayed an identical deletion pattern by fragment analysis. Strikingly, B.1.1.7 was absent in our patient population before January 2021, but prevalence has steadily increased each week. Using this method, we estimate the real-time, weekly prevalence rate to be 28.5% (8/28) in the last week of the study (3/8/21-3/12/21, FIG. 1F) despite falling total case rates in north Texas. This is consistent with the trend seen in the United Kingdom.

### Example 2

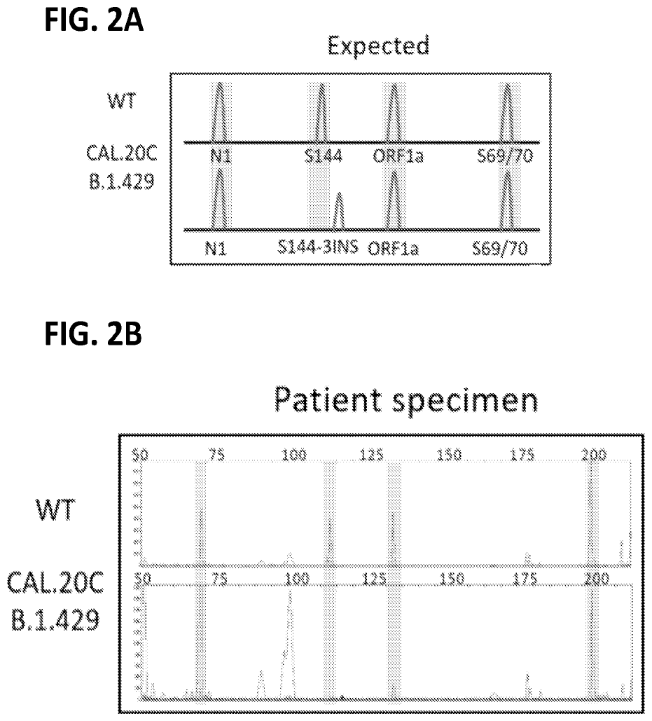

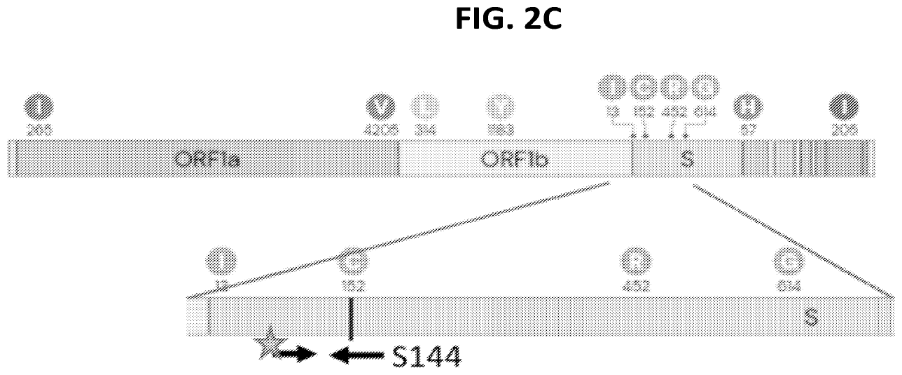

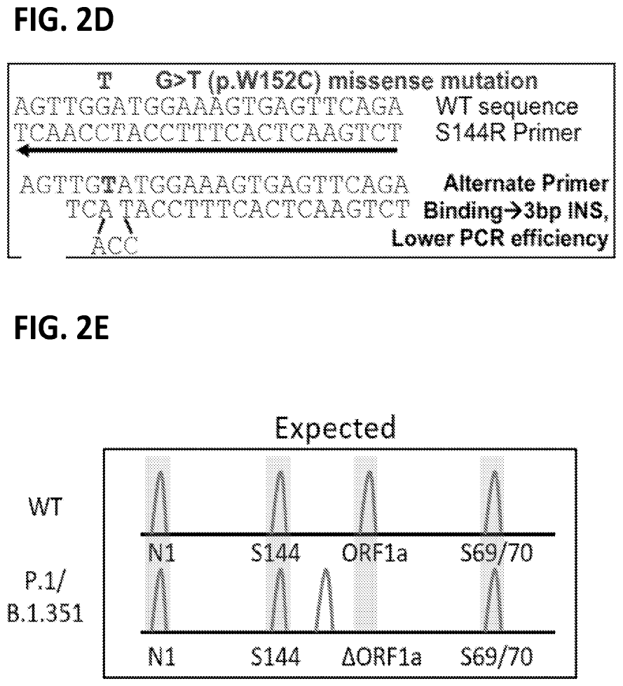

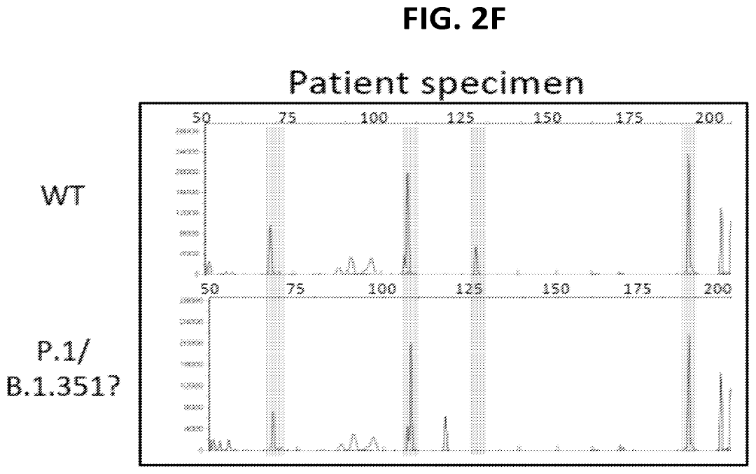

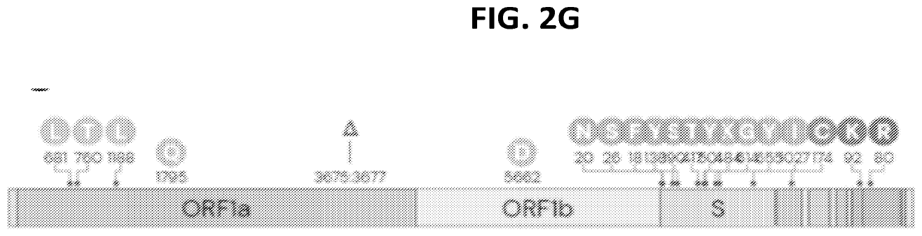

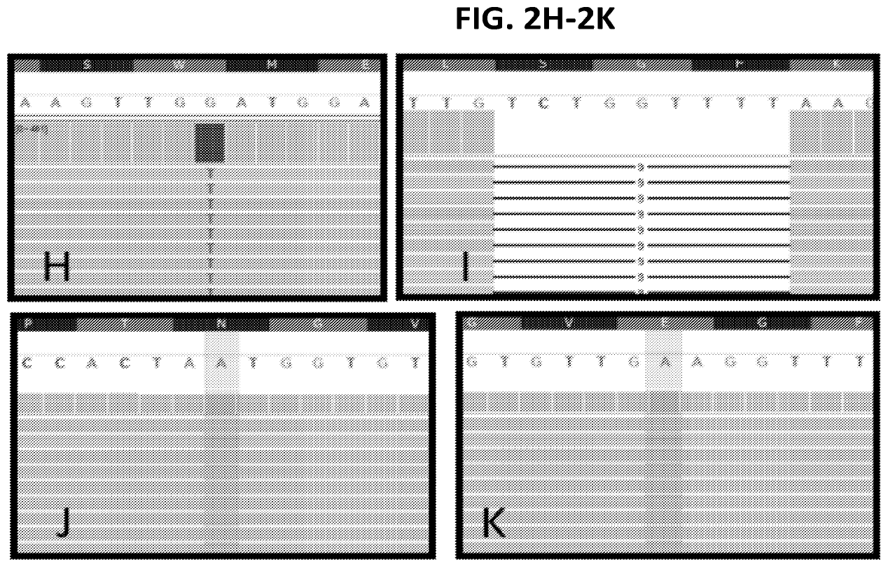

During validation studies, fragment analysis patterns unique from those associated with B.1.1.7 or wild type virus were also observed. One of these included a 3-nucleotide insertion of the S144 amplicon with dampened amplification (FIG. 2A, B). Interestingly, an emerging strain in the US (California variant, B.1.429, CAL.20C, later designated Epsilon) has a characteristic mutation (S:W152C), which overlaps with the 6^{th} nucleotide 5′ from the 3′ end of the S144 reverse primer used in the work (FIG. 2C). However, the mechanism for the 3 base pair insertion was unclear.

WGS confirmed the identity of the isolate as B.1.429 along with the W152C mutation (FIG. 2H). Alternative primer binding explains the assay-specific phenomenon. The (3-nucleotide insertion) when the 3′ end of the primer loops out the 4^{th}-6^{th} nucleotides to create an alternate binding configuration (FIG. 2D). Thus, this assay has the unexpected ability to track the B.1.429 (Epsilon) in addition to B.1.1.7 (Alpha) using only the limited number of primers reported here.

The P.1 (Gamma) and B.1.351 (Beta) variants share an ORF1A deletion identical to B.1.1.7 (Alpha), but lack S gene deletions (FIG. 2E, 2G). Therefore, when we detected an isolated 9 nucleotide deletion in the ORF1A target, these VOCs were suspected (n=6). Interestingly, although WGS confirmed the same ORF1A deletion (FIG. 2I) no other mutations characteristic of the VOC were detected (S: N501Y, E484K, FIG. 2J, 2K). Half (n=3) were classified as the B.1.526 (New York, designated as Iota) variant by PANGOLIN indicating that the ORF1A deletion may represent convergent evolution as it independently arises in geographically disparate locations. Our future directions include adding 2-3 additional characteristic markers of P.1 (Gamma)/B.1.351 (Beta) to distinguish these strains, which are currently circulating at a low level in the United States. This assay can therefore detect mutations consistent with clinically important SARS-CoV-2 strains, as well as monitor for emerging strains with related genetic alterations. These experiments are described in the following examples.

Concordant WGS results suggest genotyping 3 unique loci may be sufficient for variant classification of B.1.1.7 (Alpha). A similar principle may be true for other strains with unique genetic mutations. Additionally, the high rate of mutational change within the SARS-CoV-2 spike protein may lead to serendipitous findings, similar to the original SGTF and three nucleotide insertion of the S144 target with the California variant (B.1.259, Epsilon) described in this work.

### Discussion of Example 1 and 2

In summary, these example shows how fragment analysis may be applied to viral variant detection and epidemiology for the first time. This assay can detect characteristic viral genomic changes found in B.1.1.7 (UK, alpha), P.1 (Brazil, Epsilon), B.1.351 (South African, Beta), and B.1.529 (California, Epsilon) lineages. This approach is affordable, multiplexable (up to 20-40 primer pairs can be added), and simple to implement within clinical laboratories with molecular biology skills and instrumentation. Real-time monitoring of SARS-CoV-2 VOCs could impact epidemiological tracking and guide clinical management prior to administering monoclonal antibody therapy.

### Example 3

In this example, a PCR test is described to detect a variant (spike protein N501Y) common to 4 independent emerging strains of SARS-CoV-2 (UK (Alpha), South Africa (Beta), Brazil (Gamma), and Ohio (Delta). This mutation is present in the binding site where the spike protein binds ACE2 receptor to enter into lung cells. Studies have shown the mutation increases the binding strength to increase infectivity. This example of convergent evolution indicates this variant will be important for monitoring known and emerging SARS-CoV-2 variants.

Allele specific primers have been designed to attach specifically to the mutant (N501Y) allele (mutation) (see Table 8). This allele specific primer will be used to detect variants of SARS-CoV-2 by various PCR methods including RT-qPCR, agarose gel electrophoresis, and Capillary electrophoresis on a Sanger sequencer.

Additionally, primers have been designed to detect the wild type allele, but not the variant allele (N501Y). These primers will be used in combination to detect either the WT or mutant variants. Each of the WT and mutant primers are forward primers and both use a common reverse primer designated “common.” Two common reverse primers have been generated and form different sized amplicons of either 214 nucleotides or 230 nucleotides.

The forward primers (described below) are labeled with fluorescent molecules FAM (for WT) or HEX (for mutant). Thus, the PCR products can be separated by capillary electrophoresis on a Sanger sequencer easily to determine if a variant is present or not. This primer design is planned to be used along with other primers designed and validated by others to detect other SARS-CoV-2 mutations (S69/70del, S144del, ORF1A 3675-3677del, see Examples 1 and 2) that are specific to the UK (Alpha), South African (Beta) or Brazilian (Gamma) variants. An N1 CDC primer pair is included to ensure detection of virus. The primers for these other mutants can detect the presence of a mutant by using fragment analysis where the size of an amplified PCR fragment will be smaller when a deletion mutation is present (see Examples 1 and 2).

Two primer designs are used:

1) On the primer, substitution of T>A one base pair upstream (5′ direction) of the terminal (most 3′) nucleotide that binds either the WT or mutant allele. This design is stable enough to bind either the WT or the mutant variant if present, but destabilizes the WT primer binding the mutant sequence and destabilizes the mutant primer from binding the WT sequence. This primer is designated with a “2′TA” at the end of the name.

2) On the primer, substitution of C>A two base pairs upstream (5′ direction) of the terminal (most 3′) nucleotide that binds either the WT or mutant allele. This should confer specificity to the mutant allele by the mutant primer. This primer is designated with a “3′CA” at the end of the name.

Lastly, for optimal binding efficiency, a second version of each primer is made by removing the most 5′ nucleotide. This alters binding strength, which may improve the reaction. This change is designated by a “0.2” at the end of the name.

Primers designed according to these criteria are described in Table 16, below.

TABLE 16

| Name | Sequence (SEQ ID NO) | Tmº C. | CG% | nt | Position |
|---|---|---|---|---|---|
| Common_230_R | cagtagtgtcagcaatgtctctgcc | 68 | 52 | 25 | |
| | (SEQ ID NO: 9) | | | | |
| N501WT | 5′FAM-caatcatatggtttccaacccacta | 66.5 | 40 | 25 | c.22935- |
| | (SEQ ID NO: 10) | | | | 22959 |
| UTSW_N501Y_3CT | 5′HEX-caatcatatggtttccaacccattt | 67.3 | 36 | 25 | |
| | (SEQ ID NO: 11) | | | | |
| UTSW_E484WT_2′TC | 5′FAM-ggtagcacaccttgtaatggtgtcg | 69.3 | 52 | 25 | |
| | (SEQ ID NO: 12) | | | | |
| UTSW_E484K | 5′HEX- | 68.5 | 46.2 | 26 | c.23245- |
| | CGGTAGCACACCTTGTAATGGTGTT | | | | 23221 |
| | A (SEQ ID NO: 13) | | | | |

The primers are tested in various combinations, using capillary electrophoresis, RT-PCR, and/or fragment analysis as described above against control material containing the N501Y variant to determine the optimal combination of primer pairs.

### Example: 4 Multiplex Fragment Analysis for Flexible Detection of all SARS-CoV-2 Variants of Concern

### Introduction

SARS-CoV-2 variants of concern (VOCs) have emerged harboring epidemiologically significant genetic mutations impacting transmission kinetics, vaccine responses, mortality and monoclonal antibody therapy. Rapid identification of SARS-CoV-2 VOCs are therefore of exigent need in clinical and public health arenas. Whole-genome sequencing (WGS) remains the current gold standard for SARS-CoV-2 variant identification, however broad adoption is challenging due to requirements for specialized equipment and bioinformatics expertise. While these limitations may be mitigated through centralized high-volume testing, consequent increased turnaround time for batching, data deconvolution, and sequence analysis often exceeds an actionable timeframe for contact tracing or patient assessment.

In this example a rapid, cost-effective, high throughput multiplex fragment analysis assay named CoVarScan is described that can detect SARS-CoV-2 VOCs in a routine clinical setting. Fluorescently labeled RT-PCR amplicons are analyzed by capillary electrophoresis on a Sanger sequencer, and SARS-CoV-2 VOCs are identified by unique mutation signature. Fragment analysis accurately detects multiple deletions/insertions, and single nucleotide polymorphisms (SNPs) with alternately labeled primers. CoVarScan is scalable to high-throughput requiring approximately 4 hours to perform. Additionally, the method features modest reagent requirements, utilizing instrumentation already found in many routine clinical and public health laboratories.

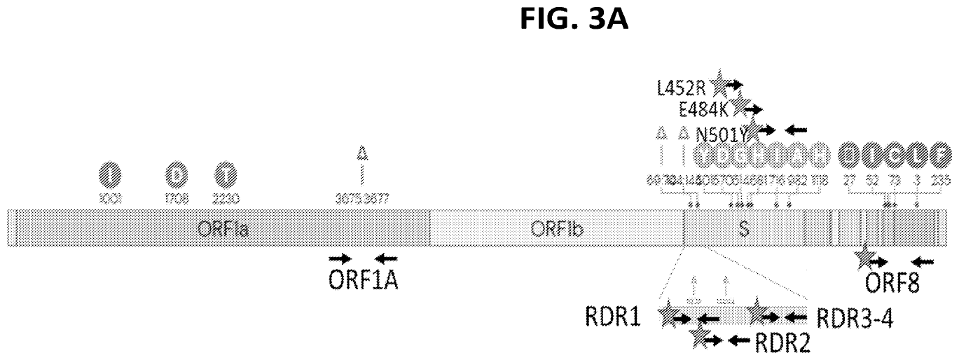

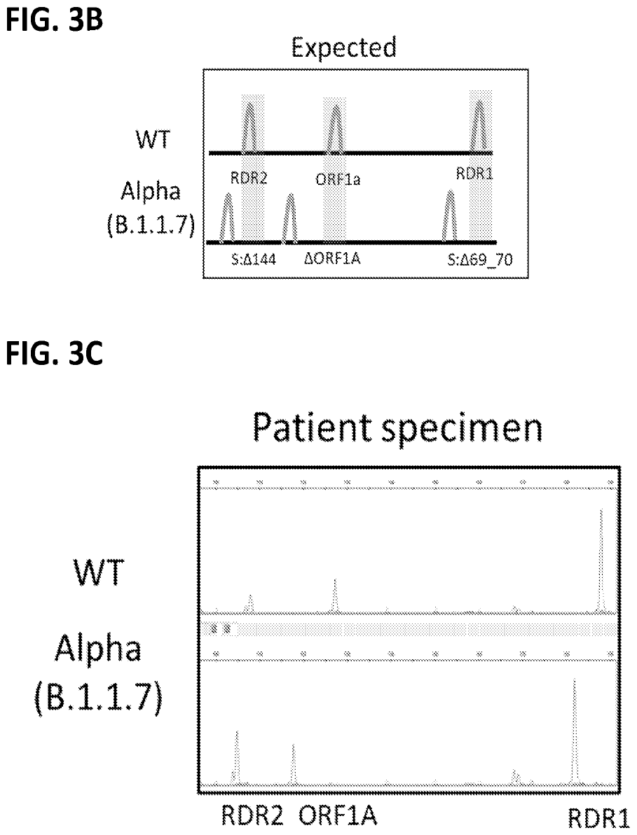

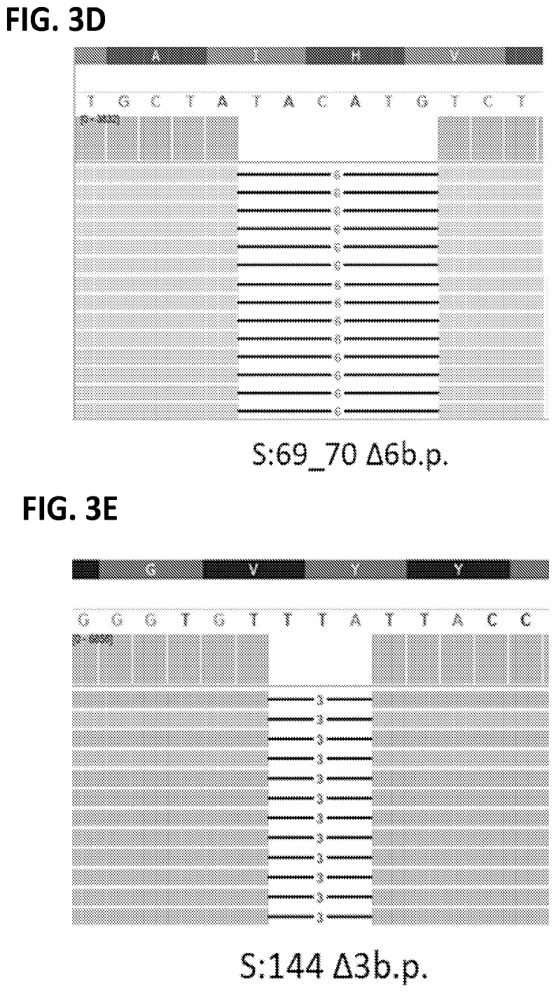

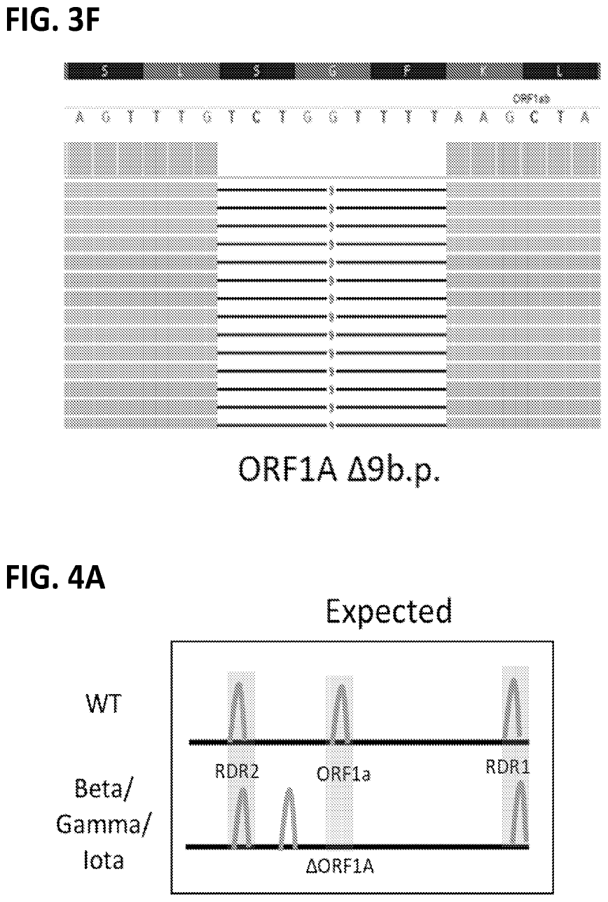

While fragment analysis could theoretically accommodate over 20 targets using different combinations of florescent dyes and amplicon sizes, it was found that targeting 8 hotspot regions was sufficient to differentiate all currently described SARS-CoV-2 variants of interest (VOIs) and VOCs. SARS-CoV-2 variants display convergent evolution with certain regions recurrently mutated. This insight was leveraged to target 5 recurrently deleted regions (S:RDR1, S:RDR2, S:RDR3-4, ORF1A, and ORF8) and 3 SNPs (S:N501Y, S:L452R, and S:E484K), which permits differentiation of all VOCs (FIG. 3A). Since its initial inception, CoVarScan has been optimized for best primer melting temperatures, PCR annealing temperature, SNP primer specificity, and balanced primer concentrations. Ideal conditions are described in Table 11 above, reproduced here.

TABLE 11
10X Primer formulation

| Primer | Quantity | | Quantity | |
|---|---|---|---|---|
| RDR1_F: (Thermo_S-F) Fwd Primer (100 uM stock) | 5 | μl | 2 | μl |
| RDR1_F: (Thermo_S-F) Rev primer (100 uM stock) | 5 | μl | 2 | μl |
| UTSW_ORF1A_F + 1 Forward Primer (100 uM stock) | 2.5 | μl | 1 | μl |
| UTSW_ORF1A_R + 5 Reverse primer (100 uM stock) | 2.5 | μl | 1 | μl |
| UTSW_ORF8_F Forward Primer (100 uM stock) | 10 | μl | 4 | μl |
| UTSW_ORF8_R Reverse primer (100 uM stock) | 10 | μl | 4 | μl |
| UTSW_RDR3-4_F Forward Primer (100 uM stock) | 7 | μl | 2.8 | μl |
| UTSW_RDR3-4_R Reverse primer (100 uM stock) | 7 | μl | 2.8 | μl |
| Yale_144del-F Forward Primer (100 uM stock) | 2.5 | μl | 1 | μl |
| UTSW_RDR2_R Reverse primer (100 uM stock) | 2.5 | μl | 1 | μl |
| UTSW_N501_WT 5′FAM-Forward Primer (100 uM stock) | 10 | μl | 4 | μl |
| UTSW_N501Y_3CT HEX-Forward Primer (100 uM stock) | 10 | μl | 4 | μl |
| UTSW_E484_2′C Forward Primer (100 uM stock) | 10 | μl | 4 | μl |
| UTSW_E484K_3′G Forward Primer (100 uM stock) | 10 | μl | 4 | μl |
| UTSW_L452_WT3′G Forward Primer (100 uM stock) | 5 | μl | 2 | μl |
| UTSW_L452R_MUT Forward Primer (100 uM stock) | 5 | μl | 2 | μl |
| UTSW_Common_230_R Reverse primer (100 uM stock) | 25 | μl | 10 | μl |
| Nuclease Free Water | 371 | μl | 148.4 | μl |
| Total | 500 | μl | 200 | μl |

The core assay detects two spike deletion mutations (S:Δ69_70-RDR1 and S: Δ144-RDR2) initially described in the Alpha VOC, and a deletion in ORF1A (ORF1A:Δ3675_3677) present in Alpha, Beta (South African, B.1.351), Gamma (Brazil, P.1), Iota (New York, B.1.526), Lambda (Peru, 0.37), and Omicron (B.1.1.529). CoVarScan was expanded to include additional allele-specific primers to detect recurrent RBD mutations (S:N501Y, S:E484K, and L452R) conferring either antibody resistance or increased transmission by higher ACER2 binding affinity. Lastly, primers flanking ORF8-N gene junction and RDR3-4 were added to differentiate Beta (S:Δ242_244) and Gamma VOCs (1ns28269-28273). Sensitivity, specificity, stability were established by comparison of all results to paired WGS.

While undertaking this work, novel lineages have emerged, but CoVarScan has not required modification since April 2021. Importantly, the core set of primers, detects and differentiates Delta, Lambda, Mu, and now Omicron VOCs. We have implemented this fragment analysis PCR assay in parallel with whole genome sequencing for routine surveillance of all COVID-19 positive specimens at our institution. CoVarScan has been optimized using over 3,000 SARS-CoV-2-positive clinical upper respiratory specimens. Variant detection has also been designed to include mutations with evidence of convergent evolution.

### General Methods

Clinical specimens. Positive nasopharyngeal specimens in viral transport media (Remel, Thermo Scientific, Lenexa, KS) from the pathology service line at the University of Texas Southwestern Medical Center were utilized for assay verification. SARS-CoV-2 positivity was determined by either RT-PCR (Alinity m SARS-CoV-2, Abbott Molecular, Des Plaines, IL, Xpert® Xpress SARS-CoV-2, Cepheid, Sunnyvale, CA) or isothermal amplification (IDNOW, Abbott Diagnostics, Scarbourough, ME). A total of 3,328 prospective specimens (4/11/21-11/2/21) SARS-CoV-2-positive specimens collected from asymptomatic, symptomatic, and confirmed COVID-19 patients were utilized. Included specimens had RT-qPCR CT values of <35 (Abbot Alinity M SARS-Cov-2 assay, Abbott Park, III) or a positive qualitative result from either the Cepheid Xpert Xpress SARS-CoV-2/Xpert Xpress SARS-CoV-2/flu (Cepheid, Sunnyvale, CA) or IDNOW COVID-19 assay (Abbott Diagnostics, Scarbourough, ME). Details regarding WGS method and bioinformatic pipeline are detailed in supplementary methods. This work was reviewed and approved by the UT Southwestern Institutional Review Board and deemed not human subject research due to an exception for disease surveillance.

SARS-CoV-2 variant control material. Positive control material consisted of synthetic RNA (Synthetic SARS-CoV-2 RNA Control 14, England/205041766/2020, 16, South Africa/KRISP-EC-K005299/2020, and 17, Japan/IC-0564/2021) produced as six, 5 kb DNA fragments (Twist Bioscience, San Francisco, CA).

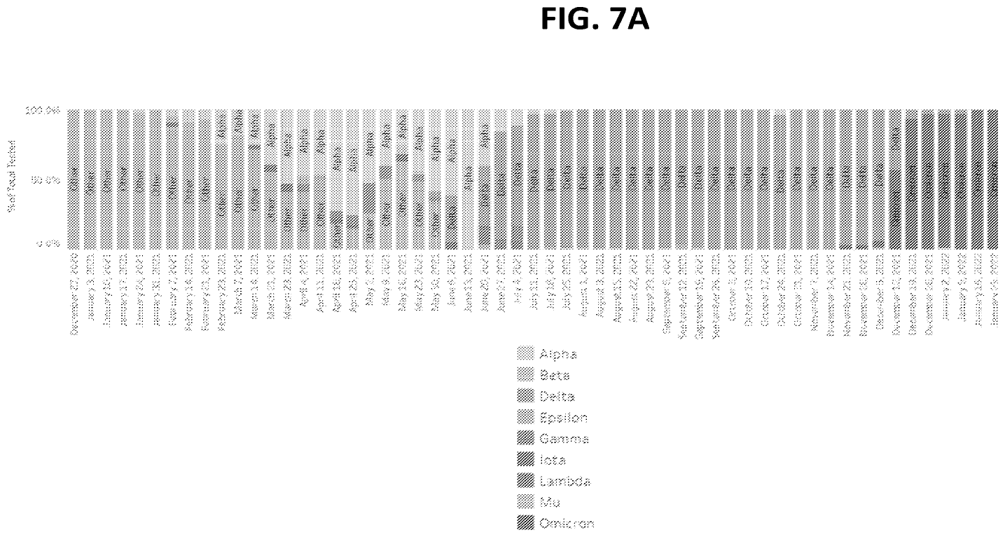

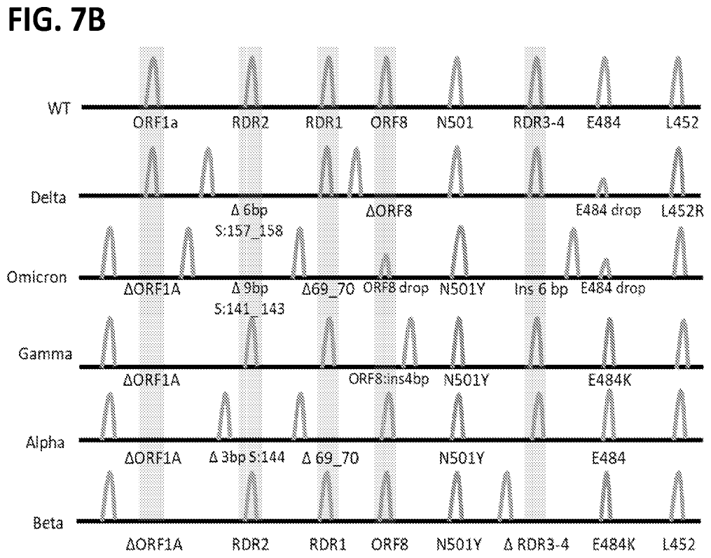

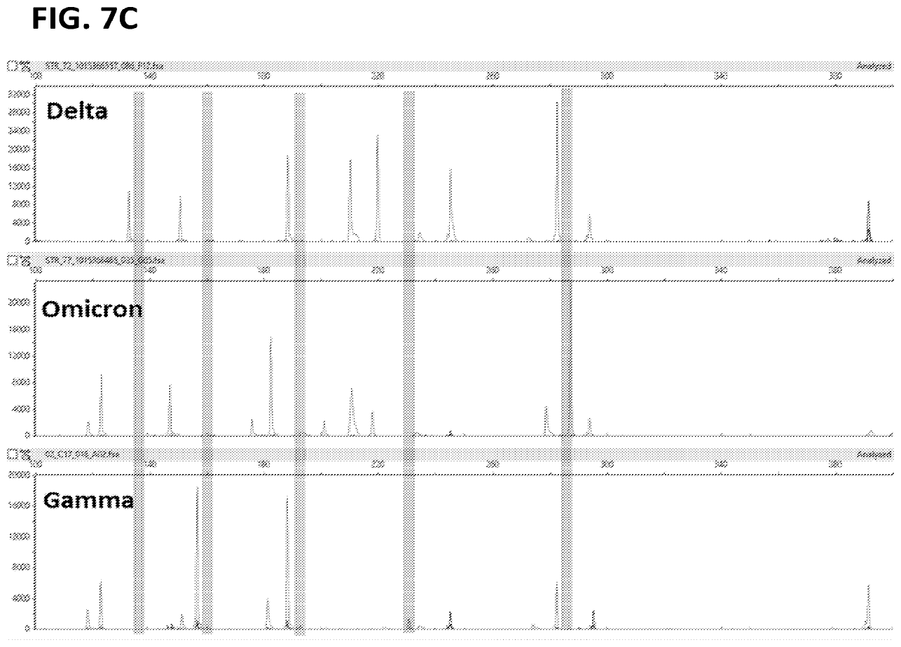

Interpretive criteria. Expected wild type and SARS-CoV-2 variant fragment lengths were confirmed (Tables 14 and 15). Samples were considered positive if capillary electrophoresis peaks were: 1) the expected size (x-axis, FIG. 1A) and 2) >50 fluorescence units (double background fluorescence). A small number of recurrent artifacts were observed (Table 15). Considering all targets together, one can identify wild type, Alpha, Beta, Gamma, Iota, Eta, Epsilon, Delta, Lambda, Mu, Omicron, and other SARS-CoV-2 variants (e.g., FIG. 7B).

### Limit of Detection

The limit of detection for each assay target was estimated using spiked Alpha, Beta and Gamma in vitro transcribed RNA (Twist Biosciences, San Francisco, CA). A twofold dilution series ranged from 2,000 to 1 copy/reaction. Specimens were analyzed in triplicate, then limit of detection confirmation was performed with 20 replicates at 100 and 5 copies/reaction.

### Methods

### RNA Extraction.

RNA extraction was performed using Chemagic™ Viral DNA/RNA 300 Kit H96 on the Chemagic™ 360 instrument (PerkinElmer, Hopkinton, MA) according to the manufacturer's protocol for high volume testing. Alternatively, RNA extraction can be performed using lower throughput or manual methods for smaller batches. A sample plate, an elution plate and a magnetic bead plate were prepared using an automated liquid handling instrument (Janus G3 workstation, PerkinElmer Inc). In brief, an aliquot of 300 μl from each sample, 4 μL Poly(A) RNA, 10 μL proteinase K and 300 μL lysis buffer 1 were added to respective wells of a 96 well sample plate. The sample plate, elution plate (60 μL elution buffer per well) and magnetic beads plate (150 μL beads per well) were then placed on the Chemagic™ 360 instrument and RNA was extracted automatically with an elution volume of 60 μL from a sample volume of 300 μL.

Next generation sequencing.

For WGS confirmation of positive results, first strand cDNA was prepared using 10 μL of RNA according to the manufacturer's instructions (Prime Script 1^{st} strand cDNA synthesis, Takara Bio, Japan). 5 μL of cDNA were converted into an NGS library using the Swift SNAP SARS-CoV-2 Kit (Integrated DNA Technologies) using tiled primer pairs in a single tube to target the entire 29.9 kb SARS-CoV-2 genome. Samples were sequenced on the Illumina® MiSeq® System at 2×250 bp coverage. WGS was performed one week after fragment analysis result review and interpretation, so reference method results were blinded from reviewers. Similarly, bioinformatic analysis was performed by a separate group who did not have access to fragment analysis test results.

### Bioinformatic Analysis.

Raw sequences were trimmed for quality and adapter sequences using TrimGalore (Felix Krueger, The Babraham Institute) with a quality threshold of 25. Trimmed reads were aligned with minimap2 to the SARS-COV-2 reference genome (NC_045512, Wuhan strain). Primer sequences were removed using primerclip (Swift Biosciences Inc.). Alignments were sorted and indexed using samtools. Duplicate reads were removed using Piccard MarkDuplicates (Broad Institute). Variants were detected using BCFtools and VarScan. Consensus sequences were determined using BCFtools. Variants were annotated using snpEFF. SARS-CoV-2 strain lineage was determined using PANGOLIN (updated on a weekly basis).

Oligonucleotides. Oligonucleotide primer sequences were are described in Table 10 above. All primers were ordered from either Integrated DNA Technologies (IDT, Coralville, IA) or Eurofins (Louisville, KY). Primers were designed and analyzed using the Multiple Primer Analyzer webtool by ThermoFisher Scientific. FAM or HEX labels were added to the 5′ end of all forward primers.

RT-PCR Amplification. RT-PCR was performed using the Thermo TaqPath 1-Step RT-qPCR kit (ThermoFisher Scientific, Waltham MA). Briefly, 2.5 μL 4× TaqPath master mix (including M-MLV reverse transcriptase and DNA polymerase), 1.5 μL nuclease-free water, and 5 μL extracted RNA were added per reaction. 1 μL of the 10× primer master mix was added per reaction, resulting in a total volume of 10 μL per reaction. This mixture was scaled to either single, 50 or 96 reactions depending on the application (Table 12, above). Single reactions were performed in standard PCR tubes, while group of 96 reactions were performed utilizing a 96-well plate. RT-PCR amplification was performed using a modification of the thermocycler settings for the CDC SARS-CoV-2 assay to include a 30-second extension step and 61° C. annealing temperature.

The RT-qPCR cycling program used for the assay is as follows: 1. Reverse transcription: 2 minutes at 25° C., 15 minutes at 50° C., 2 minutes at 95° C., 2. Amplification: 45 cycles of: 95° C. for 10 seconds, 61° C. for 30 seconds, 72° C. for 30 seconds, 3. Final extension: 72° C. for 5 minutes, terminal hold at 4° C. Following amplification, RT-PCR products were either immediately analyzed by capillary electrophoresis or stored at −20° C. protected from light until analysis could be undertaken.

Capillary Electrophoresis. Following RT-PCR, 6.5 μL Hi-Di Formamide (ThermoFisher) and 0.5 μL of GeneScan™ 500 LIZ™ standard (ThermoFisher) was added to each well of a new 96-well plate. 3 μL of RT-PCR product was added to each well, and 96-well plates were sealed with adhesive film. The plates were briefly vortexed and centrifuged gently to assure a homogenous mixture, and then amplification fragments were heat denatured at 95° C. for 3 minutes. Samples were then returned to ice and protected from light. The Sanger sequencer (Applied Biosystems 3730xl, 50 cm capillaries, Polymer POP-7™) was calibrated for the G5-RCT dye set (including FAM, VIC, and LIZ) prior to running samples, and the adhesive film was replaced with a septa on the 96-well plate containing the denatured RT-PCR products.

### Results

### Core Assay: Fragment Length Polymorphism Detection in Spike RDR1, RDR2 and ORF1A

We initially designed primers flanking the mutation (S:Δ69_70) responsible for the S-gene target failure (SGTF, ThermoFisher TaqPath assay) found in the Alpha variant (B.1.1.7, U.K. origin). This mutation is in the recurrently deleted region 1 (RDR1) of the N-terminal side of the spike gene. As the S: Δ69_70 mutation was not unique to the Alpha variant, we targeted two additional deletions: (S: Δ144) in the RDR2 gene and ORF1A: Δ3565_3567 (9 b.p. deletion). These three targets produced aberrant PCR products specific to the Alpha variant. (FIG. 3A to FIG. 3F).

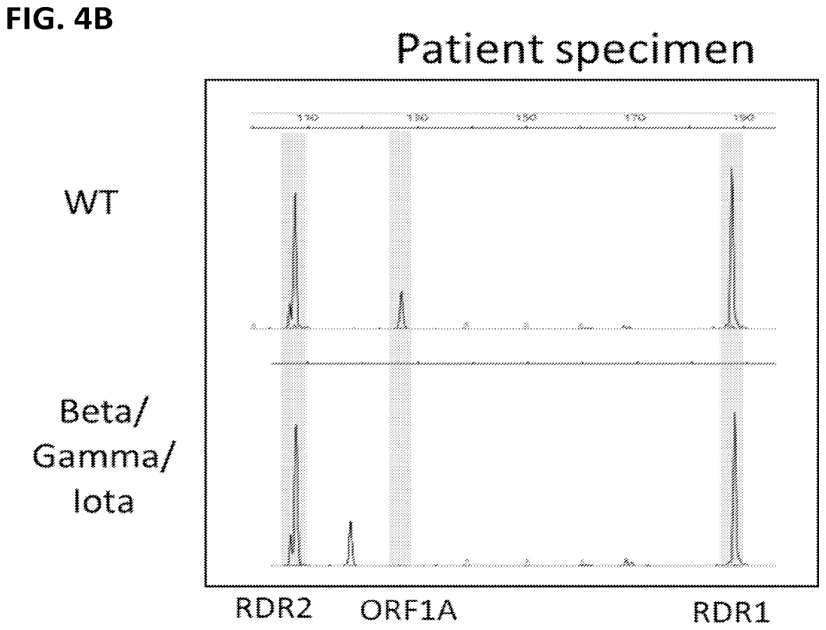

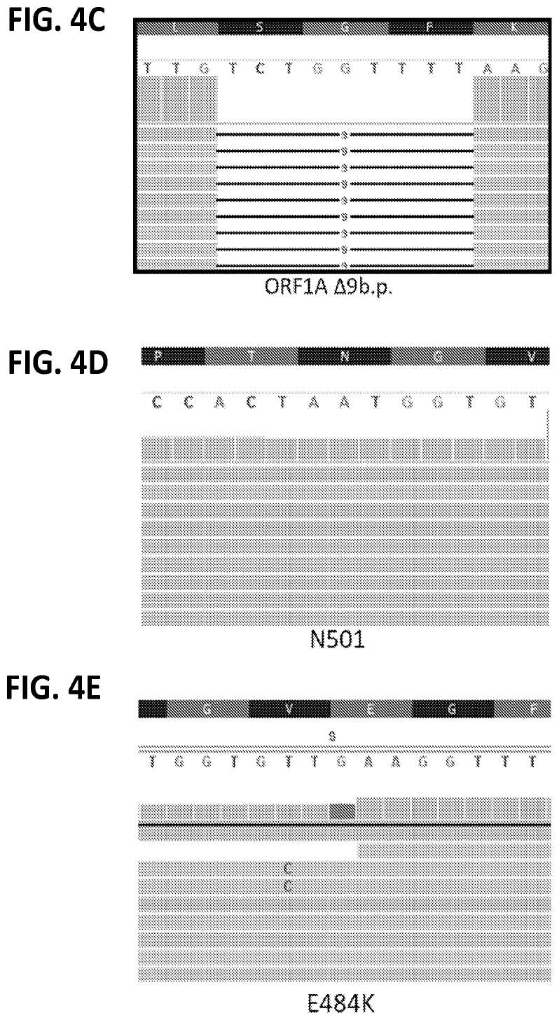

The three targets RDR1, RDR2, and ORF1A form the core assay. However, allele-specific targets were required to differentiate Beta, Gamma, and Iota variants of concern. When SARS-CoV-2 samples were tested, some contained an isolated ORF1A 9 b.p. deletion (FIG. 4A, 4B). This was originally ascribed to the Beta (B.1.351, South Africa origin) and Gamma (P.1, Brazil origin) variants, which have two receptor binding domain mutations (S:N501Y and S:E484K). However, sequencing revealed a variant with only S:E484K known as Iota (B.1.526, New York origin, FIG. 4C-4E).

### Allele-Specific Primers, S:N501Y

In additional to detecting amplicons of different size, fragment analysis can detect single nucleotide polymorphisms by either restriction digestion or allele-specific primers. We chose allele-specific primers to avoid additional handling and off target effects of restriction enzymes, in addition to the financial and technical demands for maintaining such reagents for a high-throughput assay in a routine setting.

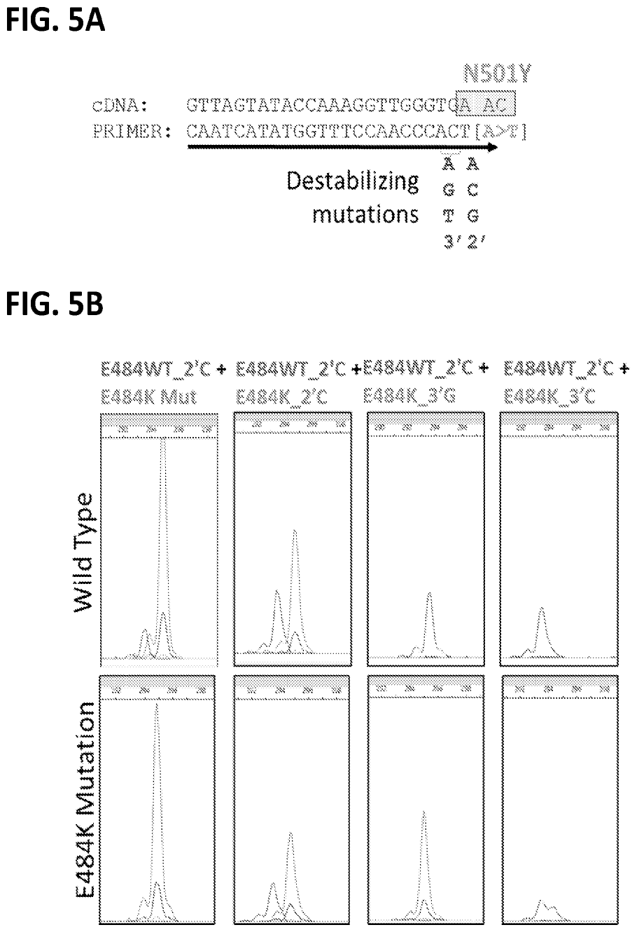

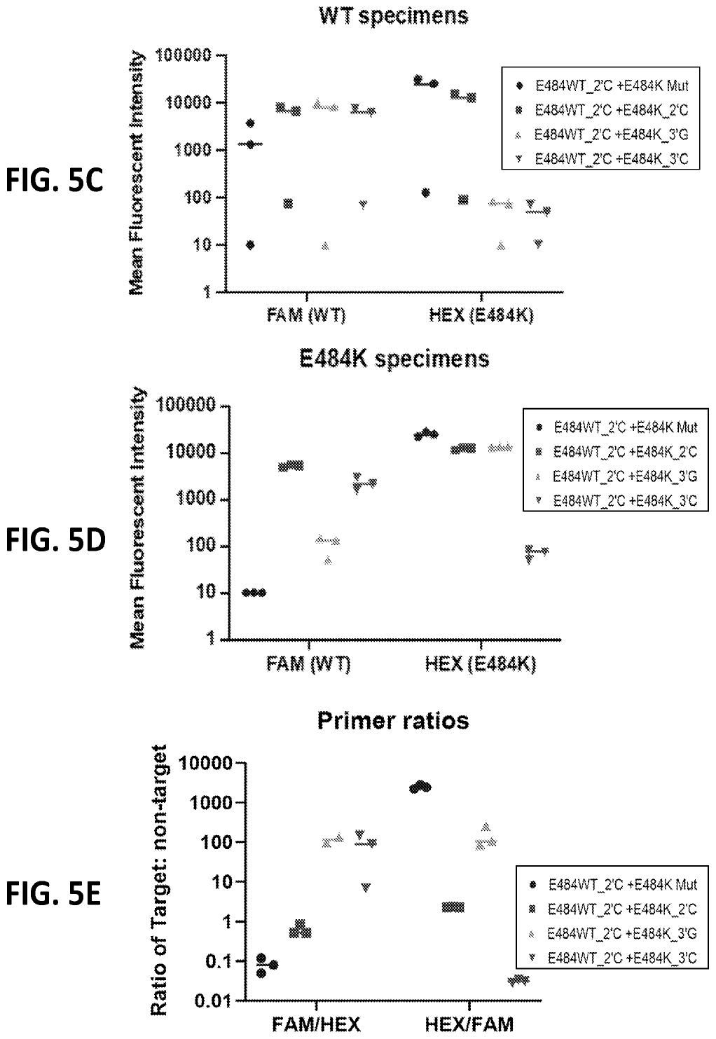

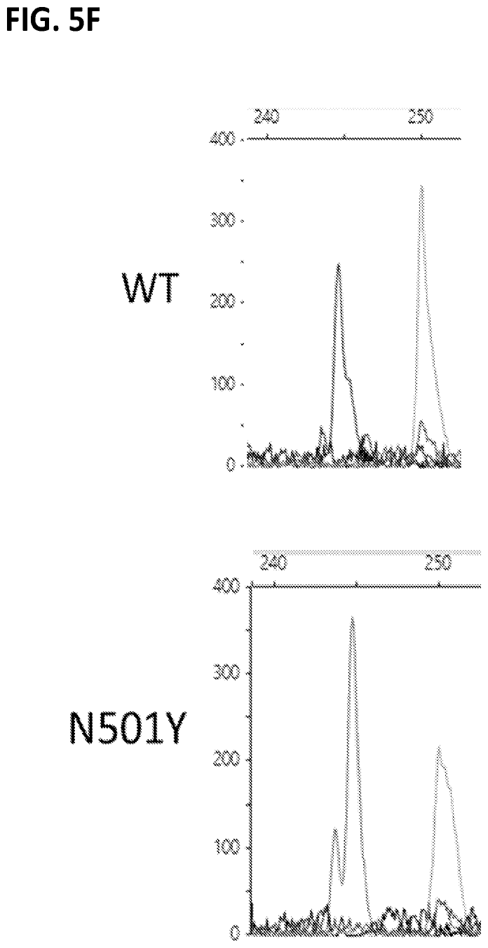

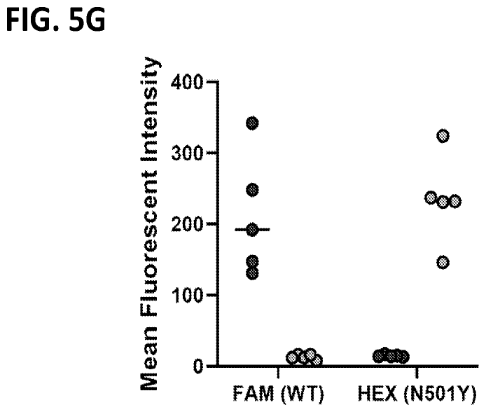

Wild type specific (FAM) and mutant specific (HEX) fluorescently-labeled primers were designed to contain terminal 3′ nucleotides specific to the target allele (FIG. 3A). To reduce cross-reactivity, mutations were added to the second to last (2′) and third to last (3′) nucleotide to destabilize improper primer binding or extension (FIG. 5A). S:N501Y was targeted as it increases ACE2R binding affinity 7-fold and was present in the first 3 VOCs described (Alpha, Beta, and Gamma) representing convergent evolution.

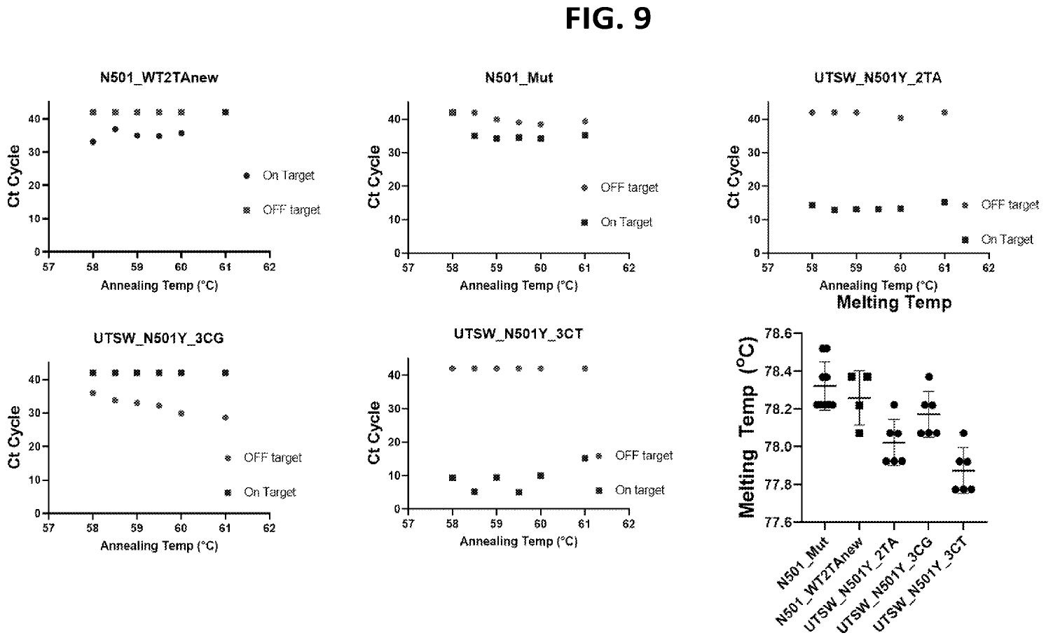

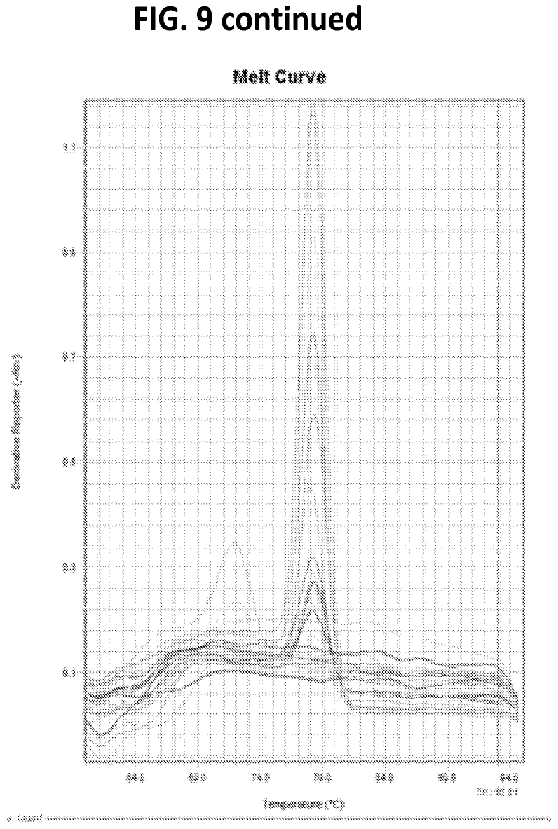

Real-time quantitative polymerase chain reaction (RT-qPCR) with SYBR green amplification detection was performed to screen primers (Table 17) for on-target vs. off-target amplification. UTSW_N501Y_3′C>T was labeled with HEX dye as it was the lead candidate with the lowest CT for mutant sequence, a single melt-curve peak, and least WT amplification across multiple annealing temperatures (FIG. 9). The N501_WT, unmutated primer, was selected for FAM dye labeling. For fragment analysis, the combination of UTSW_N501Y_3′C>T primer was labeled with HEX and UTSW_N501_WT was labeled with FAM, and this combination properly discriminated WT from mutant sequence (FIG. 5F, FIG. 5G).

TABLE 17
Optimization primers for N501Y

| | | SEQ ID | Bind | Bind Alt | >8 Ct |
|---|---|---|---|---|---|
| Primer Name | Sequence (SEQ ID NO: ) | NO | Target? | Target? | Difference |
| N501_WT2TA | Catatggtttccaacccacaa | 24 | N | | |
| N501y2TA | Catatggtttccaacccacat | 25 | N | | |
| N501_WT | Caatcatatggtttccaacccacta | 10 | | | |
| N501_Mut | Caatcatatggtttccaacccactt | 26 | Y | Y | 4 |
| N501_WT2TAnew | aatcatatggtttccaacccacAa | 27 | Y | N | 5 |
| N501_WT2TG | aatcatatggtttccaacccacGt | 28 | N | | |
| UTSW_N501Y_2TA | caatcatatggtttccaacccacAt | 29 | Y | N | 27 |
| UTSW_N501Y_2TG | caatcatatggtttccaacccacGt | 30 | N | | |
| UTSW_N501Y_3CA | caatcatatggtttccaacccaAtt | 31 | N | | |
| UTSW_N501Y_3CG | caatcatatggtttccaacccaGtt | 32 | Y | Y | 7 |
| UTSW_N501Y_3CT | caatcatatggtttccaacccaTtt | 11 | Y | N | 35 |

### Allele-Specific Primer, S: E484K

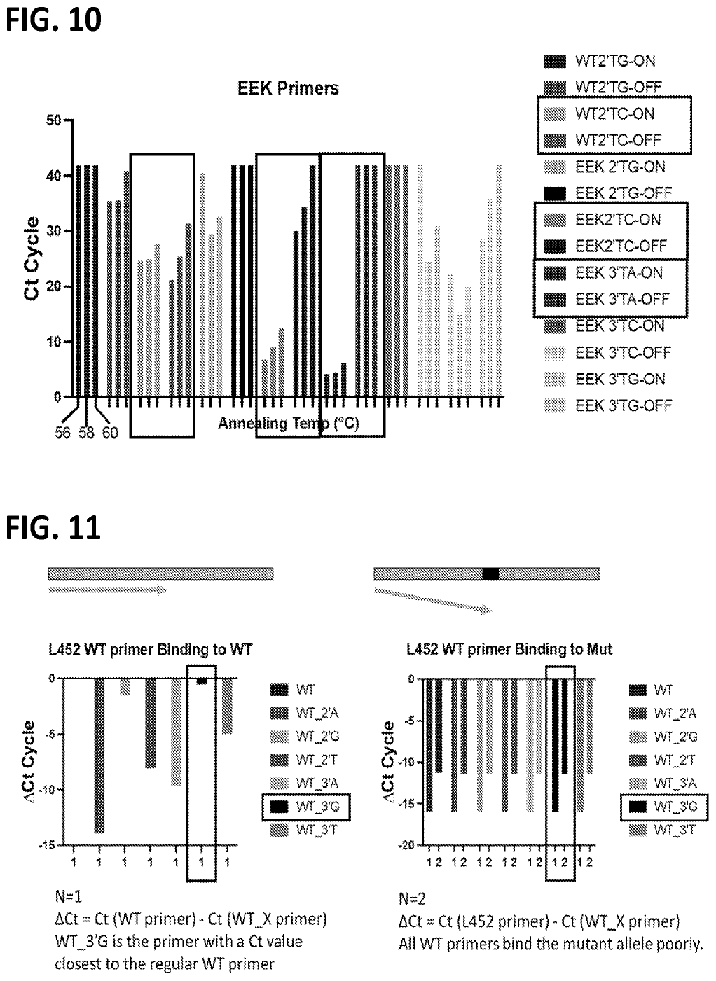

In the Beta and Gamma variants, the S:E484K mutation in the receptor binding domain aids in evasion of specific immune Reponses. As with N501Y targeting, mismatch allele-specific primers were selected for E484K (Table 18). These primers used the same common reverse primer as N501Y due to close genomic proximity. UTSW_E484_WT_2′T>C had the lowest CT value against wild type sequence (FIG. 10, WT_2′TC-ON). The WT_2′TC-OFF cross-reactive CT value was higher at 60° C. (Ct=31) compared to the mutant specific signal at 60° C., so primer UTSW_E484_WT_2′T>C was labelled with FAM. UTSW_E484K 3′TA primer amplified its intended target at a very low CT value (4-4.5) across annealing temperatures with single melt curve peaks, and amplification was undetectable for WT sequence (FIG. 10). Therefore, primer UTSW_E484K_3′TA was selected and labeled with HEX.

TABLE 18
Optimization primers for E484K

| | | SEQ ID | Binds its | Doesn′t bind |
|---|---|---|---|---|
| Name | Sequence | NO | target? | alt target? |
| WT_2′TG | ggtagcacaccttgtaatggtgtGg | 33 | N | N |
| UTSW_WT_2′TC | ggtagcacaccttgtaatggtgtCg | 12 | Y | Y |
| E484K_2′TG | ggtagcacaccttgtaatggtgtGa | 34 | Y | N |
| E484K_2′TC | ggtagcacaccttgtaatggtgtCa | 35 | Y | Less |
| E484K_3′TA | ggtagcacaccttgtaatggtgAta | 36 | Y | N |
| UTSW_E484K_3′TC | ggtagcacaccttgtaatggtgCta | 37 | N | Y |
| E484K_3′TG | ggtagcacaccttgtaatggtgGta | 38 | Y | Less |

However, when this primer combination was tested by capillary electrophoresis, HEX signal was absent when the S:E484K mutation was present, but cross-reactive FAM signal remained. Therefore, four alternative primers (E484K_Mut, E484K_2′C, E484K_3′G, and E484K 3′C) were labeled with HEX and tested against WT (n=3) and E484K specimens (n=3; Iota, New York variants, FIG. 5B). When tested against these specimens, primers were eliminated for a) amplifying WT sequence (E484_Mut, E484K_2′C, FIG. 5B, 5C) or b) not amplifying mutant sequence (E484K_3′C, FIG. 5B, 5D). The combination of E484WT_2′C+E484K 3′G produced specific signal for both WT and E484K by capillary electrophoresis (FIG. 5E); therefore, the combination of E484WT_2′C+E484K_3′G was selected for the assay.

### Allele Specific Primer S:L452R

In March of 2021, the Epsilon variant (B.1.427/B.1.429) from California emerged with a mutation (S:L154R) resistant to therapeutic antibodies (Bamlanivimab). The site is a hotspot for variation as it is also present all 3 Indian variant lineages (B.1.617.1, B.1.617.2, B.1.617.3) and an alternative mutation S:L452Q is present in the Lambda variant (C.37).

As in silico prediction models used for S:E484K allele-specific primers did not produce optimal results, we created all possible mutations in the terminal 2^{nd} and 3^{rd} nucleotides of primers (Table 19). All primers were combined with the Common reverse primer used for N501Y and E484K reactions (Common_230) and cDNA for 1) the WT or 2) S:L452R allele to compare amplification by qPCR CT value.

TABLE 19
Optimization primers for L452R

| | | | | | Binds | Doesn′t |
|---|---|---|---|---|---|---|
| | | SEQ ID | | | its | bind alt |
| Primer | Sequence | NO: | Tmº C. | CG% | target? | target? |
| FAM_UTSW_452_WT | ctaaggttggtggtaattataattaccT | 39 | 60.8 | 32.1 | Y | N |
| 452_WT2′A | ctaaggttggtggtaattataattacAT | 40 | 59.8 | 28.6 | N | N |
| 452_WT2′T | ctaaggttggtggtaattataattacTT | 41 | 59.2 | 28.6 | N | N |
| 452_WT2′G | ctaaggttggtggtaattataattacGT | 42 | 61.5 | 32.1 | Y | N |
| 452_WT3′A | ctaaggttggtggtaattataattaAcT | 43 | 59.2 | 28.6 | N | N |
| FAM_UTSW_452_WT3′G | ctaaggttggtggtaattataattaGcT | 18 | 61 | 32.1 | Y | N |
| 452_WT3′T | ctaaggttggtggtaattataattaTcT | 44 | 59.2 | 28.6 | Less | N |
| HEX_UTSW_L452R_Mut | ctaaggttggtggtaattataattaccG | 19 | 63.4 | 35.7 | Y | Less |
| L452R_2′A | ctaaggttggtggtaattataattacAG | 45 | 60.3 | 32.1 | N | N |
| L452R_2′G | ctaaggttggtggtaattataattacGG | 46 | 63.4 | 35.7 | Less | N |
| L452R_2′T | ctaaggttggtggtaattataattacTG | 47 | 60.3 | 32.1 | N | N |
| L452R_3′A | ctaaggttggtggtaattataattaAcG | 48 | 61.9 | 32.1 | Less | N |
| HEX_UTSW_L452R_3′G | ctaaggttggtggtaattataattaGcG | 49 | 63.6 | 35.7 | Y | N |
| L452R_3′T | CtaaggttggtggtaattataattaTcG | 50 | 61.9 | 32.1 | N | N |

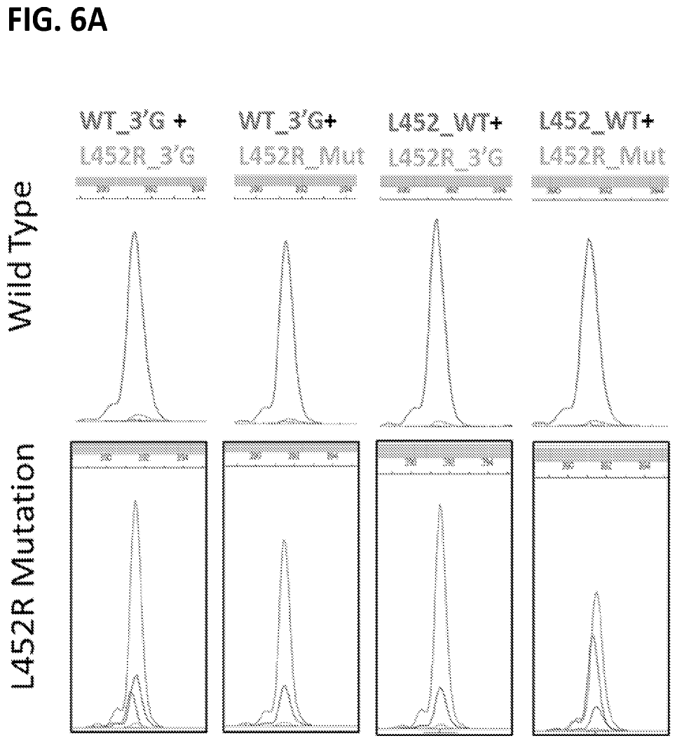

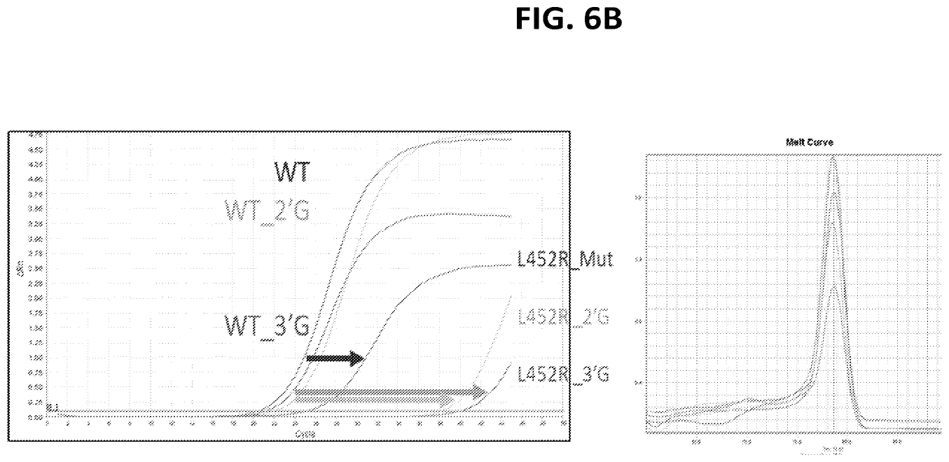

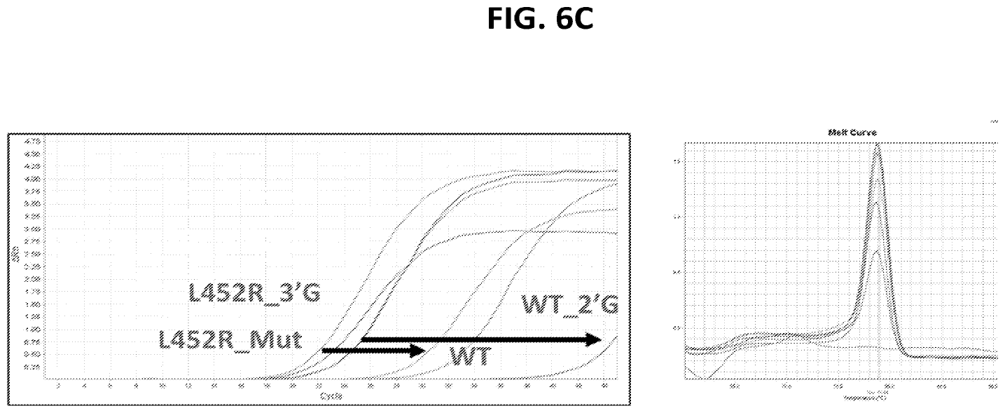

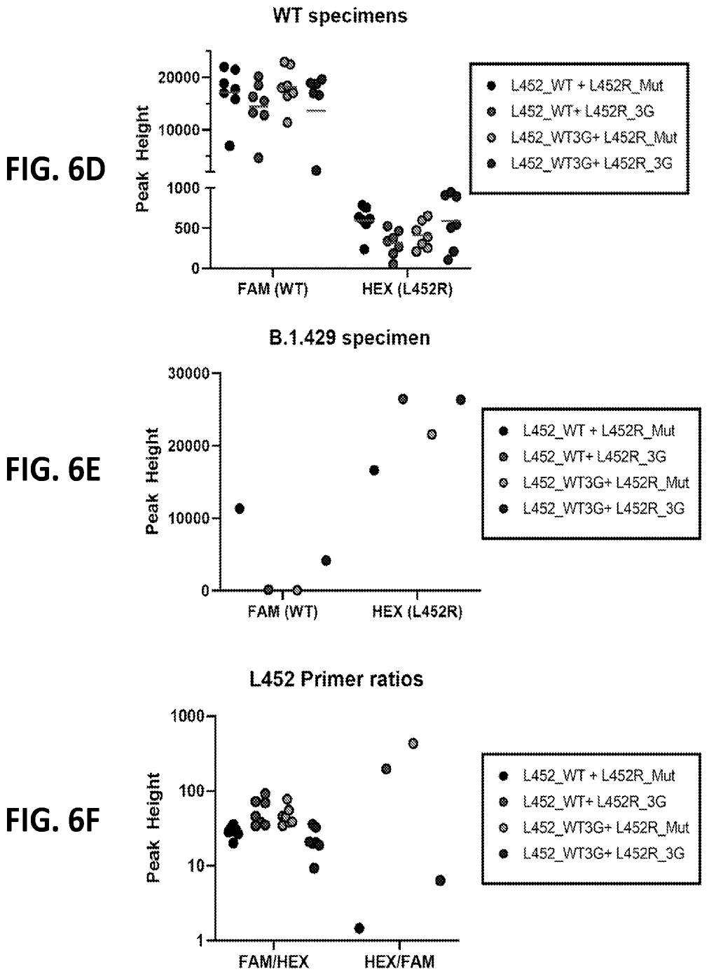

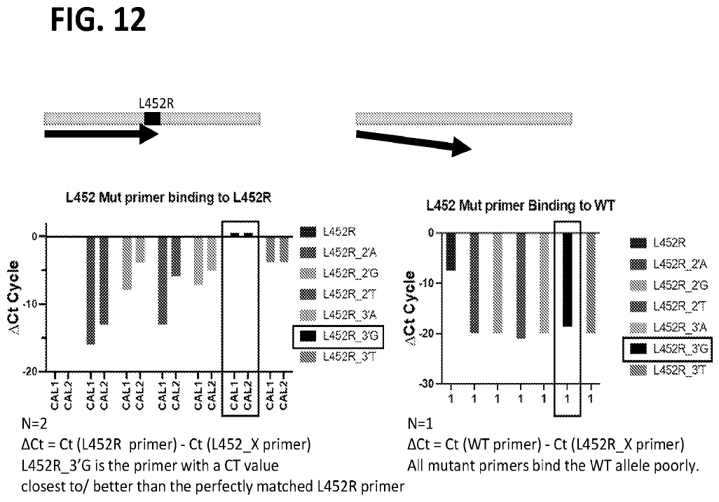

The WT 3′G primer had the lowest CT value (closest to the unmutated WT primer on-target binding) (FIG. 6E, FIG. 11). When presented with the L452R mutant, the WT-3′G primer did not amplify mutant sequence (FIG. 6F, FIG. 12). The L452R_3′G mutant specific-primer amplified mutant sequence as well as the unmutated L452R specific-primer. All mutated L452R primers (including L452R_3′G) did not amplify WT sequence (FIG. 11 and FIG. 12). Melt curve analysis confirmed all PCR reactions amplified a single PCR product (FIG. 6E and FIG. 6F).

Selected mutated primers (WT 3′G, L452R_3′G) and un-mutated (WT, L452R) primers were tested by capillary electrophoresis (FIG. 6A-6D). Four combinations were created and reacted against WT (n=7) and S1L452R sequence (n=1 Epsilon) from patient specimens. The combination of WT+L452R_3′G and WT_3′G+L452R created strong, specific WT signal (FIG. 6A). However, WT+L452R and WT_3′G+L452R_3′G were eliminated for off-target WT signal against mutant sequence (FIG. 6E). The WT_3′G+L452R combination was selected for the assay, because it had higher HEX/FAM ratio than WT+L452R_3′G (FIG. 6F).

### Discriminating Gamma (P.1) Versus Beta (B.1.351) SARS-CoV-2 Variants

The targets described so far produce an identical mutational signature in Beta and Gamma variants (ORF1A 9 bp deletion, N501Y^{MUT}, E484K^{MUT}). Thus, additional targets are required to discriminate these variants. An RDR4 spike deletion (S:Δ242_244) is present in Beta but absent in Gamma. Conversely, a 4 b.p. insertion between ORF8 and N genes (ins AAAC) is present in Gamma but absent from Beta. Thus, fluorescently labeled primers were created spanning the RDR3-4 and ORF8/N regions to create fragments 282 b.p. and 226 b.p. in size that would not interfere with other targets. Adding these ORF8 and RDR3_4 fragment analysis targets completed assay design, providing a unique mutational signature for these major VOC.

### Multiplex Optimization

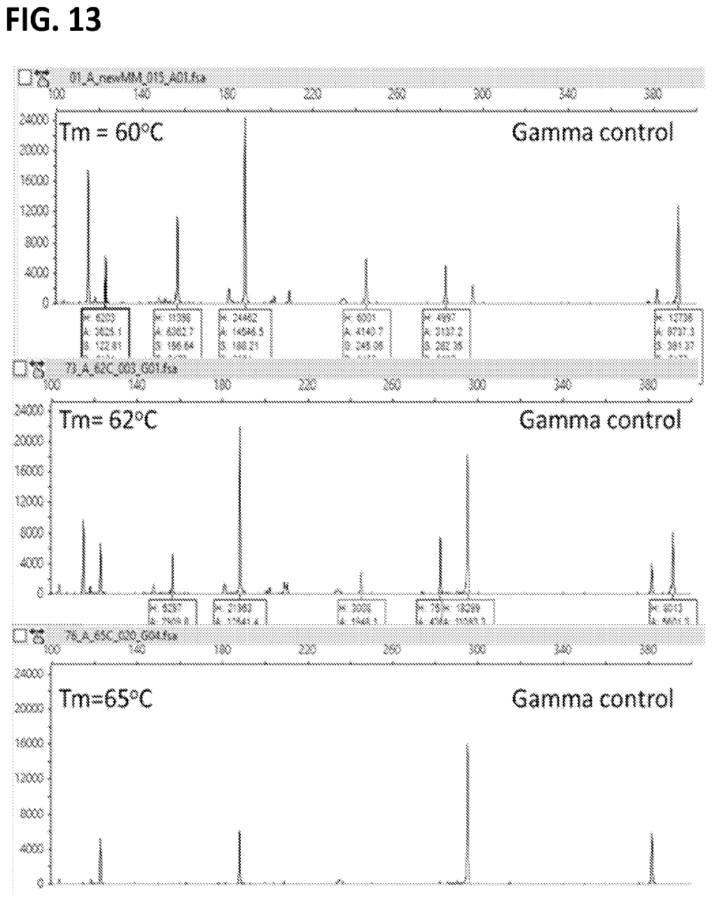

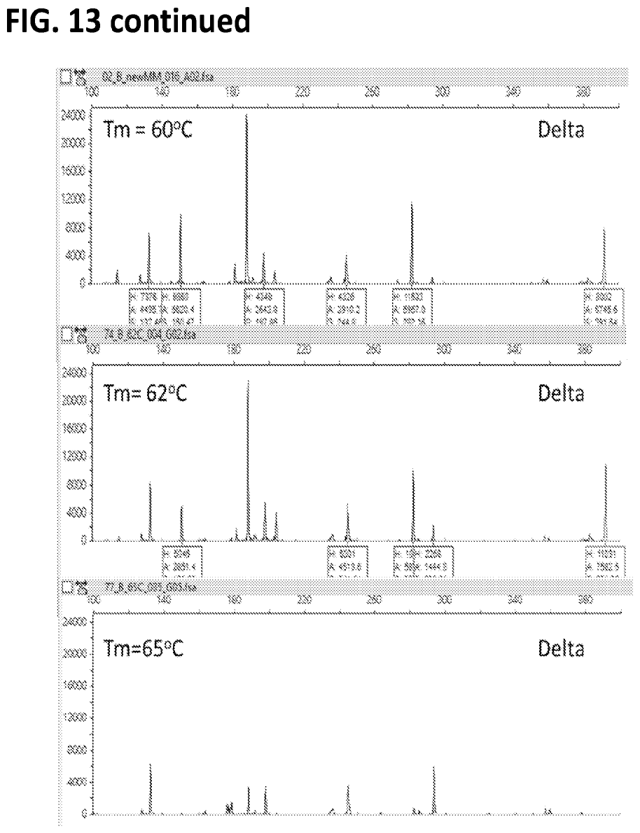

CoVarScan was optimized to balance PCR product size by annealing temperature and primer input levels. The shortest amplicons often oversaturated the fluorescent detectors while several longer PCR products had much lower intensity. To determine optimal primer concentrations, the highest peak height PCR product (RDR2 and ORF1A) primers were reduced to 50 nM, RDR3-4, ORF8, N501Y and E484K were much improved at a 200 nM primer concentrations (Table 10, above). Annealing temperatures from 60-65° C. were tested and the optimal performance was set at 61° C. (FIG. 13).

### Clinical Verification

To validate CoVarScan for clinical use, we used SARS-CoV-2 WGS as a comparative method to confirm the variants detected were accurate. Specificity was established using 34 SARS-CoV-2 Negative specimens, 60 SARS-CoV-2 positive WT specimens, and 20 non-template control (NTC) samples. WGS experienced contamination in the NTC samples (n=9), and fragment analysis detected 3 L452R and 2 N501Y mutations in the WT sample batch. Upon review, all mutations were analytically accurate. As the N501Y mutation is not variant defining, they were included and two of the L452R mutated cases were called Epsilon variants by more recent bioinformatic software, so were excluded (n=60-2). Thus only one of the L452R mutated cases was called a false positive, and the rest of the samples were negative for signal (n=34 negative samples, n=20 NTC) or negative for a variant (n=57 WT) resulting in a negative percent agreement of 99.1% (111/112, Table 20).

TABLE 20

| | NTC: | NTC: | | |
|---|---|---|---|---|
| | WGS | WGS | SARS-CoV-2 | SARS-CoV-2 |
| | Positive | Negative | Negative: EUA | Positive, WT |
| CoVarScan | 0 | 0 | 0 | 1 |
| Positive | | | | |
| CoVarScan | 9 | 11 | 34 | 57 |
| Negative | | | | |

3,238 specimens were sent for WGS, but 155 (88+67 Table 16) were excluded for not returning a lineage (total results=3,083). Positive percent agreement was 94.6% (2,917/3,083). Variants sampled included Alpha (n=125), Beta (n=3), Gamma (n=21), Delta (n=2,820), Lambda (n=5), Mu (n=6), Iota (n=6), and Omicron (n=3). The discrepancy rate was 1.1% (32/2949), which was most likely caused by variability in sensitivity of the two assays. Overall sensitivity of CoVarScan was 95.6% (2,949/3,083, Table 21).

TABLE 21

| | WGS Positive |
|---|---|
| CoVarScan Positive | 2949 |
| CoVarScan Negative | 134 |

### Limit of Detection

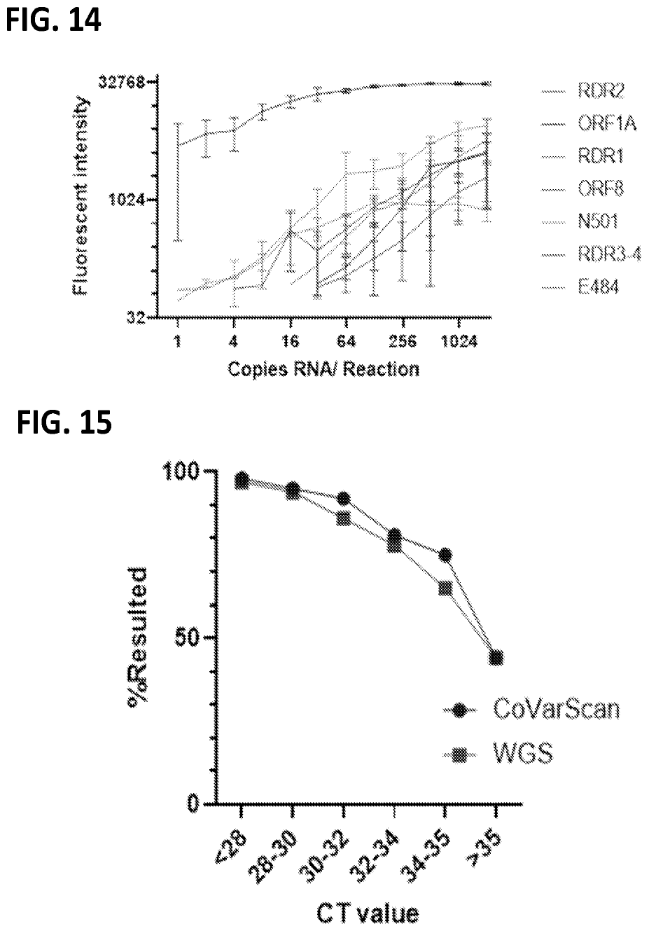

Limit of detection studies used Alpha variant control RNA in triplicate from 1000 copies/reaction to 1 copy/reaction. Triplicate testing detected all targets at 63 copies/reaction and the core 3 targets were detected at 4 copies/reaction (FIG. 14). Sensitivity was confirmed at 100 copies/reaction (full 8-plex panel) and 5 copies/reaction (core panel targets: RDR1, RDR2, ORF1A) with 20 replicates showing 95% detection for the full and core panel respectively (Table 22, FIG. 14).

TABLE 22
Limit of detection study with B.1.351 and P.1 RNA.

| Targets | ORF1A | RDR2 | RDR1 | ORF8 | N501Y | RDR3-4 | E484K | L452 |
|---|---|---|---|---|---|---|---|---|
| 1,000 | 3/3 | 3/3 | 3/3 | 3/3 | 3/3 | 3/3 | 3/3 | 3/3 |
| 500 | 3/3 | 3/3 | 3/3 | 3/3 | 3/3 | 3/3 | 3/3 | 3/3 |
| 250 | 3/3 | 3/3 | 3/3 | 3/3 | 3/3 | 3/3 | 3/3 | 3/3 |
| 125 | 3/3 | 3/3 | 3/3 | 3/3 | 3/3 | 3/3 | 3/3 | 3/3 |
| 63 | 3/3 | 3/3 | 3/3 | 2/3 | 3/3 | 3/3 | 3/3 | 2/3 |
| 31 | 3/3 | 3/3 | 3/3 | 0/3 | 1/3 | 1/3 | 0/3 | 0/3 |
| 16 | 3/3 | 2/3 | 3/3 | 0/3 | 1/3 | 1/3 | 0/3 | 0/3 |
| 8 | 3/3 | 2/3 | 3/3 | 0/3 | 0/3 | 0/3 | 0/3 | 0/3 |
| 4 | 3/3 | 3/3 | 3/3 | 0/3 | 0/3 | 0/3 | 0/3 | 0/3 |
| 2 | 3/3 | 0/3 | 1/3 | 0/3 | 0/3 | 0/3 | 0/3 | 0/3 |
| 1 | 3/3 | 0/3 | 1/3 | 0/3 | 0/3 | 0/3 | 0/3 | 0/3 |

TABLE 23

| Concentration | ORF1A | RDR2 | RDR1 | ORF8 | N501Y | RDR3-4 | E484K | L452 |
|---|---|---|---|---|---|---|---|---|
| 100 copies/ | 20/20 | 20/20 | 20/20 | 20/20 | 19/20 | 19/20 | 19/20 | 19/20 |
| reaction | | | | | | | | |
| 5 copies/ | 20/20 | 20/20 | 20/20 | 0/20 | 20/20 | 16/20 | 16/20 | 14/20 |
| reaction | | | | | | | | |

### Method Comparison of 8-Plex PCR to WGS

PCR is often more sensitive compared to massively parallel sequencing methods, and we compared the ability to classify variants by 8-plex fragment analysis vs. WGS at various CT values. Percent positivity for both methods was >90% for CT <32 dropping to 74% for the PCR method at a CT value of 34-35. (Table 24, FIG. 15)

TABLE 24

| Summary | PCR+ | WGS | Total |
|---|---|---|---|
| <28 | 2020 | 2004 | 2061 |
| 30-28 | 105 | 104 | 111 |
| 30-32 | 109 | 102 | 118 |
| 32-34 | 82 | 79 | 101 |
| 34-35 | 49 | 42 | 65 |
| >35 | 34 | 34 | 77 |
| | 2399 | 2365 | 2533 |
| <28 | 98% | 97% | 1106 |
| 30-28 | 95% | 94% | 64 |
| 30-32 | 92% | 86% | 78 |
| 32-34 | 81% | 78% | 59 |
| 34-35 | 75% | 65% | 48 |
| >35 | 44% | 44% | 27 |
| Total | 95% | 93% | 100% |

### Emerging Variants

A common issue facing variant genotyping approaches is they must be adaptable to emerging variants. We hypothesized that targeting mutational hot spots with fragment analysis was sufficient to evaluate mutational signatures specific to any SARS-CoV-2 variant (FIG. 7B, Table 25). Thus, assay design was frozen in April 2021 and evaluated variant detection and discrimination from May 2021-November 2021 as novel SARS-CoV-2 variants emerged (FIG. 7A).

TABLE 25

| Target | Size | Interpretation | B.1.1.7 | B.1.351 | P.1 | B.1.617.2 | B.1.429 | B.1.525 | B.1.526 | C.37 | B.1.1.529 |
|---|---|---|---|---|---|---|---|---|---|---|---|
| RDR2 | 108 | 9 b.p. | 0 | 0 | 0 | 0 | 0 | 0 | 0 | 0 | 88.2 |
| | | deletion | | | | | | | | | |
| RDR2 | 111 | 6 b.p. | 0 | 0 | 0 | 89.1 | 0 | 0 | 0 | 0 | 0 |
| | | deletion | | | | | | | | | |
| RDR2 | 113 | 3 b.p. | 94.9 | 1.1 | 0.3 | 0.1 | 0.3 | 92.6 | 21.2 | 0.7 | 0 |
| | | deletion | | | | | | | | | |
| ORF1A | 117 | 9 b.p. | 96.8 | 90.4 | 96.6 | <0.1 | 0.1 | 95.6 | 98.0 | 95.9 | 90.9 |
| | | deletion | | | | | | | | | |
| RDR1 | 182 | 6 b.p. | 97.0 | 0.1 | 0.3 | 0.2 | 0.7 | 93.6 | 0.6 | 0.7 | 89.1 |
| | | deletion | | | | | | | | | |
| RDR3_4 | 173 | 9 b.p. | <0.1 | 75.1 | <0.1 | <0.1 | <0.1 | 0.1 | <0.1 | 0 | 0 |
| | | deletion | | | | | | | | | |
| RDR3_4 | 191 | 9 b.p. | 0 | 0 | 0 | 0 | 0 | 0 | 0 | 0 | 87.4 |
| | | insertion | | | | | | | | | |
| ORF8 | 197 | 6 b.p. | <0.1 | <0.1 | <0.1 | 94.3 | 0 | 0 | <0.1 | <0.1 | 0 |
| | | deletion | | | | | | | | | |
| ORF8 | 209 | 4 b.p. | 0 | 0 | 95 | 0 | 0 | 0 | 0 | 0 | 0 |
| | | insertion | | | | | | | | | |
| N501Y | 244 | Mutant | 97.5 | 86.6 | 96.3 | 0.1 | 0.1 | 0.1 | 0.1 | 0.1 | 92.9 |
| E484K | 293 | Mutant | 0.3 | 85.7 | 96.0 | <0.1 | 0.1 | 97.1 | 55 | 0.1 | 0 |
| L452R | 392 | Mutant | 0.1 | 0.5 | <0.1 | 97.6 | 96.2 | <0.1 | 22.2 | L452Q | 0 |
| | | | | | | | | | | 97.7 | |

On April 11^{th}, B.1.617.2 (delta) was the first variant to be encountered that was not accounted for in 8-plex assay design. However, we found a characteristic mutational signature: RDR2^{Δ6 b.P.}+ORF8^{Δ6 b.P.}+S:L452R^{Mut}+S:E484^{drop out}. S:E484 target drop out was attributed to the S:478K mutation overlapping the E484 primer binding site. The RDR2 deletion matches the S:Del157_158 mutation (FIG. 7C), and the ORF8 deletion matches the ORF8:Del119_120 mutation characteristic of Delta variant.

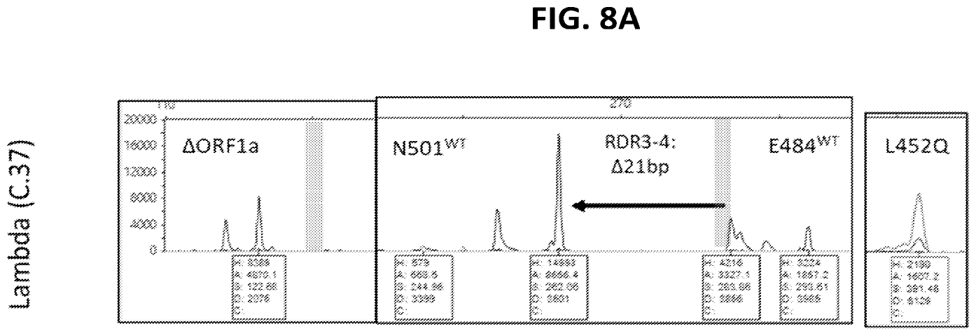

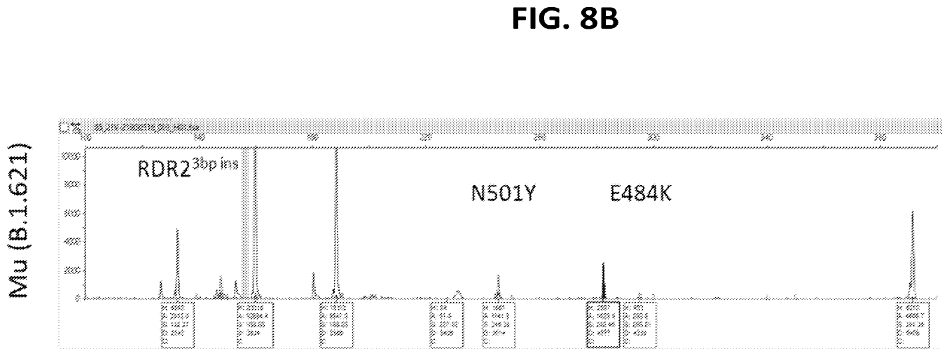

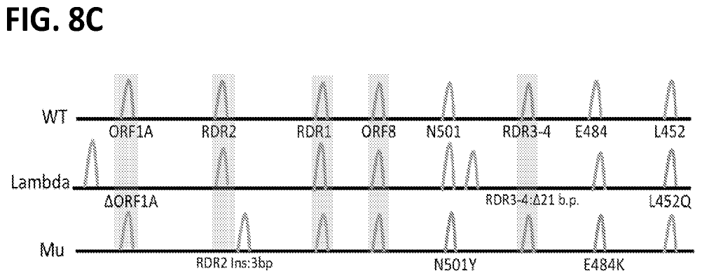

Next, in June, the Lambda variant from Peru (C.37) was found in five specimens and was defined by the signature of ORF1^{Δ6 b.P.}+RDR3_4^{Δ21 b.P.}+L452R^{Mut}. Although Lambda has an L452Q mutation present, the mutant specific primer cross-reacts and is able to contribute to the unique mutation signature (FIG. 8A, and FIG. 8C).

The Mu variant (B.1.621) from Colombia was concerning due to similar RBD mutations as Beta and Gamma when found in July, but could be distinguished by its unique mutational signature: RDR2^{Ins3 b.p.}+N501Y^{Mut}+E484K^{Mut} (FIG. 8B, and FIG. 8C). This insertion at RDR2 indicates the RDR regions are prone not only to deletions, but insertions too. This further demonstrates a key feature of the fragment analysis assay to detect PCR amplicons of different sizes in hotspot regions.

Lastly, the Omicron variant is the most recent variant with concern for increased spread and immune resistance. The key mutations created a unique signature that was predicted (FIG. 7B), observed (FIG. 7C) and WGS confirmed. The mutational signature is ORF1A^{Δ9 b.P.}, RDR2^{Δ9 b.P.}, RDR1^{Δ6 b.P.}, RDR3-4^{ins6 b.p.} and N501Y^{Mut}. The characteristic deletion pattern was detected in three cases of Omicron and allowed early reporting and tracking by the public health department.

In total, positive percent agreement is 94.6% concordant with Whole genome sequencing. Thus, in the last several months, CoVarScan has discriminated SARS-CoV-2 variants with accuracy similar to whole genome sequencing (Table 26).

TABLE 26

| CoVarSc | SARS-COV-2 WGS (Reference) | | | | | | | | | Below |
|---|---|---|---|---|---|---|---|---|---|---|
| an PCR | WT | Alpha | Beta | Gamma | Delta | Lambda | Mu | Iota | Omicron | LOD |
| WT | 25 | 9 | | | 4 | | | | | 3 |
| Alpha | 4 | 125 | | | 3 | | | | | 6 |
| Beta | | | 3 | | | | | | | |
| Gamma | | | | 20 | 1 | | | | | |
| Delta | 3 | 1 | | | 2724 | | | | | 79 |
| Lambda | | | | | | 5 | | | | |
| Mu | | | | | 1 | | 6 | | | |
| Iota | 2 | 1 | | | | | | 6 | | |
| Other | | | | | 1 | | | | 3 | |
| Variant | | | | | | | | | | |
| Below | 19 | 28 | | 1 | 86 | | | | | 67 |
| LOD | | | | | | | | | | |

Total cases: 3,238
Exclude WGS Below LOD results (n = 155): 3,083
Positive Percent Agreement: 25 + 125 + 3 + 20 + 2724 + 5 + 6 + 6 + 3/3,083 = 94.6%
Discrepancy rate: Discrepant results/ total results = 32/2949 = 1.1%
$Sensitivity = \frac{True Positives}{\begin{matrix}True Positives + \\ False Negatives\end{matrix}} = \frac{2 , 949}{2 , 949 + 134} = 95.6 \%$
$Specificity = \frac{True Negatives}{\begin{matrix}True Negatives + \\ False Positives\end{matrix}} = \frac{111}{111 + 1} = 99.1 \%$

### Discussion

Here we have successfully conceptualized, created, and implemented a novel fragment analysis-based approach to detect SARS-CoV-2 positive nasopharyngeal specimens for VOCs in a clinical setting. Among the advantages are decreased time to result, adaptability to new variants without modification, and use of non-complex instrumentation and analysis.

Prospectively, the application of a reliable method for VOC determination provides economic advantages. In the United States, the Delta VOC became dominant in July 2021. In practice, if a specimen was identified as Delta (or any other dominant variant) by this assay, it would not require sequencing as WGS is primarily used for the detection of emerging variants. Thus, screening for epidemiologically dominant VOCs using a cost-effective method allows conservation of WGS resources for investigation of non-dominant VOC lineages, and would enable deeper variant monitoring of COVID-19 positive samples. Based upon these estimates, when the prevalence of a VOC exceeds 40-50%, it is more cost effective to screen samples routinely by fragment analysis with reflexive WGS for non-predominant lineages compared to prospective WGS on all specimens.

Fragment analysis differs from RT-PCR-based screening methods for VOCs in a number of ways. While several non-sequencing approaches have been described to screen for SARS-CoV-2 VOCs, they lack the flexibility and scalability of fragment analysis. Some RT-PCR approaches rely upon allele-specific molecular beacon probes that discriminate between wild type and mutant alleles by identifying shifts in melt curves or dot plot populations in ddPCR. While some commercial assays have successfully multiplexed up to four targets in a single reaction, fluorescent channels are limited to 4-5 channels in most real-time PCR platforms. Similarly, multiplex RT-qPCR has been described for detection of VOCs by targeting the ORF1A^{Δ9} b.p. and RDR2^{Δ3 b.p.} deletion. However, while these tests are useful for screening, the method provided herein can classify a variant with similar accuracy as sequencing without target modification. Furthermore, assay throughput may be lower (and more expensive) if multiple wells are required to test enough targets for classification.

Additionally, some RT-qPCR assays detect variants by target loss such as with S-gene target failure in the ThermoFisher TaqPath assay. This method has been used in the United Kingdom to track the prevalence of B.1.1.7 and now Omicron variants. However, there are SGTF cases that arise sporadically. In contrast, this assay gives improved specificity by measuring the size of multiple deletions. Mutational signatures of the 8 targets can be as epidemiologically specific as WGS. This feature differentiates fragment analysis from other approaches that merely screen for variants. For example, the epidemiologic specificity of ORF1AA among the most prevalent variants with ORF1AA mutations is 88% (567,563/643,640) S: Δ144 is 98% (553,578/563,775) and S: Δ69_70 is 96% (572,995/594,454). Considered together, these mutations are 98% specific for Alpha, differentiating it from other SARS-CoV-2 strains that may have one of the other deletions.

CoVarScan has high-throughput capacity as it can be performed in a single well with 96 tests run at a single time by capillary electrophoresis. Result Interpretation requires approximately 1 hour for a 96-well plate, but can be automated using PCR fragment size-specific “bins” in the GeneMapper Software. This is an important feature as genotyping variant assays have emerging clinical utility.

The assay has sensitivity similar to higher than most WGS platforms (CT <28-30). With an increasing number of targets, primer dimers may form; however, the length of these dimers should all be <70 b.p., so they should not interfere with classifying the current targets of this assay. Some recurrent artifacts have been found, but they do not overlap with any targeted PCR amplicons (Table 15). This test cannot find all possible SNPs that may occur. Some mutations such as the S:K417N mutation in “Delta Plus” (AY.1/AY.2) were concerning for antibody resistance, but remained <0.1% of total Delta lineages.

In summary, we have applied fragment analysis to SARS-CoV-2 VOC detection for the first time. This assay can detect characteristic viral genomic changes found in all variants at single nucleotide resolution. This cost-effective, rapid assay, and scalable assay has been extensively tested and performs as accurately as WGS, which supports its use for both rapid variant surveillance and clinical decision making if significant resistance to an antibody therapy is found.

### Example 5: Tracking Omicron, “Stealth Omicron” (BA.2) and Beyond without Sequencing

The CoVarScan was performed on samples containing the Omicron variant and the Stealth Omicron (BA.2). The official name is BA.2 but is called “stealth” omicron because it lacks the S:Del69_70 mutation responsible for S-gene target failure (SGTF in the TaqPath COVID-19 assay). S-gene target failure is used as a rapid surrogate test to track the rise of omicron variants.

The BA.2 variant is starting to rise in several locations (Denmark, Philipines, Singapore), and may represent increased transmissibility compared to the original omicron strain (BA.1). Multiple sub-lineages are not unusual; the Delta variant (B.1.617.2) was one of 3 sub-lineages arising from India at the same time. However, given differing spike protein mutations, BA.2 could have alternative monoclonal antibody therapeutic options.

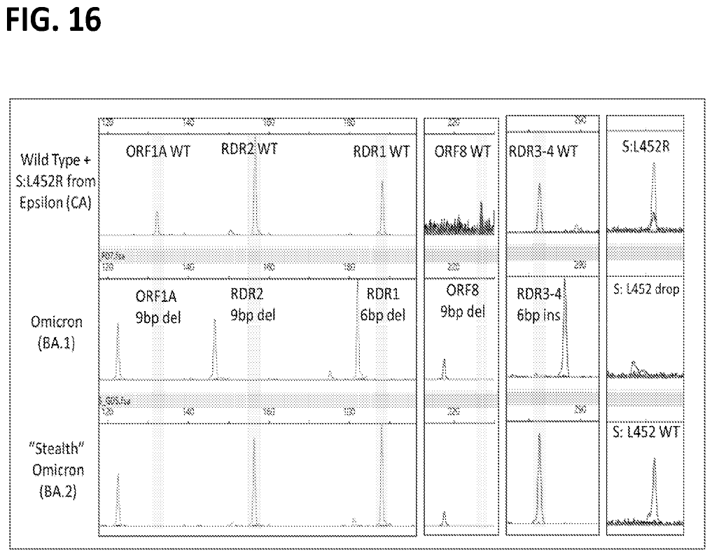

Using the CoVarScan protocol described in previous examples, a sample containing BA.2 was identified with the distinct mutational signature described in Table 27 and shown in FIG. 16.

TABLE 27

| Omicron (B.1.1.529/BA.1) | | Omicron (BA.2) | |
|---|---|---|---|
| | CoVarScan | | CoVarScan |
| Mutation | Result | Mutation | Result |
| ORF1A: Del | ORF1A: 9 bp del | | ORF1A: 9 bp del |
| 3675_3677 | | | |
| N: Del 31_33 | ORF8: 9 bp del | N: Del 31_33 | ORF8: 9 bp del |
| S: Del 69_70 | RDR1: 6 bp del | None | RDR1: WT |
| S: Del 143_145 | RDR2: 9 bp del | None | RDR2: WT |
| S: Del 211 + | RDR3-4: 6 bp ins | None | RDR3-4: WT |
| S: Ins 214EPE | | | |
| S: L446 | L452: drop | None | L452: WT |

Table 1. Mutational differences between BA.1 and BA.2

A common critique of SNP based variant genotyping assays is that they require adjustment for new variants. However, this method has required no design adjustment since pre-Delta and has identified unique mutational signatures for each variant.

### Example 6—Detection of Co-Infection of Multiple Strains of SARS-COV-2 in Single Individual

### Introduction

Ongoing genomic surveillance has revealed the trajectory of severe acute respiratory syndrome coronavirus 2 (SARS-CoV-2) infections largely coincide with the emergence of dominant genetic lineages (variants) harboring distinct genetic mutations. SARS-CoV-2 variants of concern (VOCs) have arisen, harboring a high number of mutations and increase infectivity, morbidity, immune evasion, and mortality among infected individuals. Evolutionarily, the SARS-CoV-2 mutates relatively slowly compared to HIV or influenza virus owing to the presence of nonstructural protein 14 (Nsp14) that harbors 3′-to-5′ exoribonuclease (ExoN), a unique RNA proof-reader not found in other RNA viruses. Genome-wide association studies (GWAS) indeed indicate that mutations in error-correcting nsp14 protein exhibited a strongest association with increased SARS-CoV-2 genome-wide mutation load. Other factors can also change the rate of evolution as VOCs rise to high frequencies.

For instance, when multiple variants circulate, the possibility of genomic recombination between different strains or sub-lineages increases. Recombination occurs when two distinct lineages are present in the same host. Importantly, these novel genotypes are concerning for the ability to create a virus with the worst aspects of each variant in a short amount of time. Indeed, this was recently observed to occur when the Omicron variant out-competed the Delta VOC. In addition, severely immunocompromised (IC) hosts with no antibody immune response have been observed to harbor the virus for up to a year with no increased mutation rate. However, some IC hosts may exhibit an immune response low enough to prevent viral clearance, but that is high enough to prompt viral evolution. Such a scenario has been observed in uncontrolled HIV patients with low but detectable CD4 counts or other transplant patients.

While a combination of genetic drift, recombination, and population immunity could play a key role in the continual emergence of VOCs, reasons underlying this emergence remain poorly understood. In the face of population immunity, it has been previously reported that the IC patients elude vaccines or remain more pathogenic (8-10). Further, Sun et al. (11) have shown that IC individuals exhibit a higher risk of COVID-19 breakthrough infection. Of note, weakened immune system was determined to be a potential source for multiple amino acid substitutions and deletions in the spike protein, with increased resistance to neutralizing antibodies (5). Thus, these studies suggest IC states prolong viral survival and lead to acquisition of additional mutations. It is thus critical to address whether underlying immunodeficiency or immunosuppressive therapy impacts the emergence of recombinant variants by permitting co-infection of variants.

Typically, molecular tools such as whole genome sequencing (WGS) have facilitated evolutionary and epidemiological monitoring of SARS-CoV-2 in near real-time and enabled us to better prepare and combat the threat. Detection and characterization of co-infection and emerging recombinant variants by current WGS methods is challenging with respect to obtaining and analyzing complete viral genomes from clinical samples and overcoming cost and labor. However, genotyping methods allow sensitive detection of multiple mutations simultaneously, which can provide a clue to the existence of co-infection. Using fragment analysis genotyping method for detecting SARS-CoV-2 variants (12), two cases of a Delta/Omicron co-infection and two cases of an Omicron/BA.2 co-infection were detected. These findings were confirmed by WGS.

The present example presents two cases of SARS-CoV-2 Delta/Omicron (B.1.617.2/B.1.1.529) co-infection, and the first two cases of co-infection with BA.1/BA.2 Omicron sub-lineages among IC patients. The knowledge of co-infections is critical and will enforce an evidence-based understanding of the genome changes and the patterns of infection, thereby will help to maximize the impact of management and treatment of COVID cases.

### Case Description and Clinical Presentation of the Lung Transplant Patient

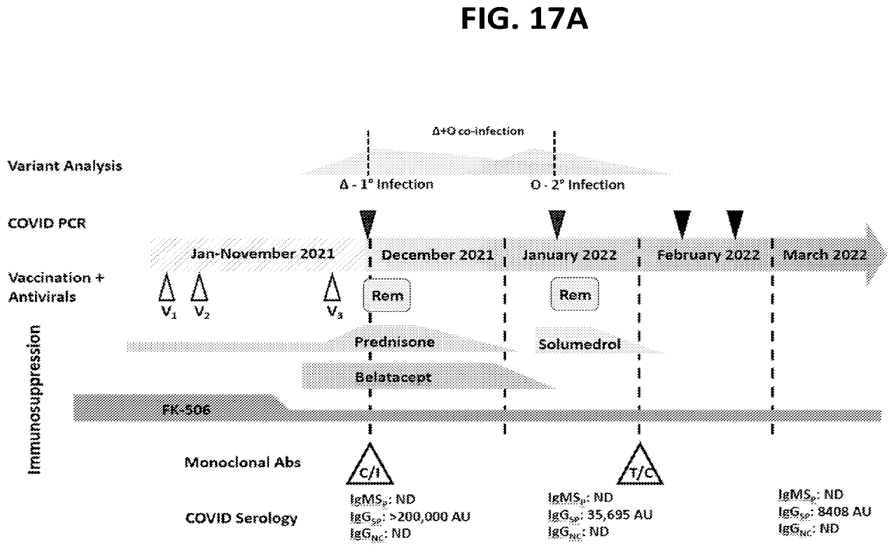

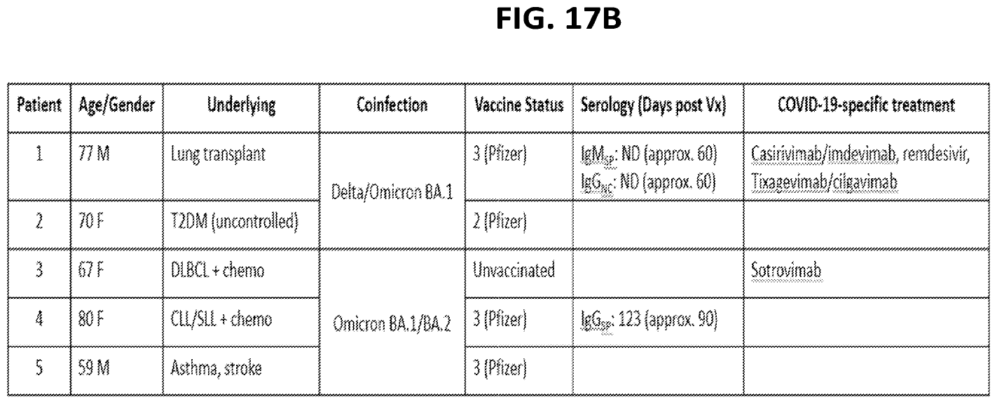

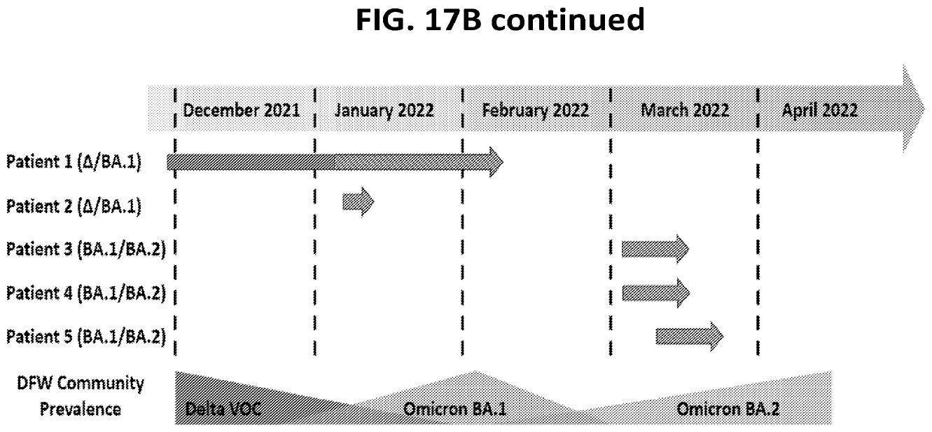

A 76-year-old male with an 8-year-old lung transplant (due to idiopathic pulmonary fibrosis) presented with fever and respiratory symptoms in November 2021 (FIG. 17). SARS-CoV-2 PCR was positive and SARS-CoV-2 genotyping identified mutations consistent with the Delta variant. The patient's outpatient immunosuppression regimen included prednisone, tacrolimus, and monthly belatecept infusions. He had received three doses of the Pfizer/BioNTech COVID-19 vaccine prior to presentation. The patient was initially treated with Casirivimab/Imdevimab (Regeneron) followed by dexamethasone and remdesivir for 5 and 10 days, respectively. He was discharged without supplemental oxygen but reported continued exertional dyspnea and reduced forced expiratory volume (FEV1) from baseline.

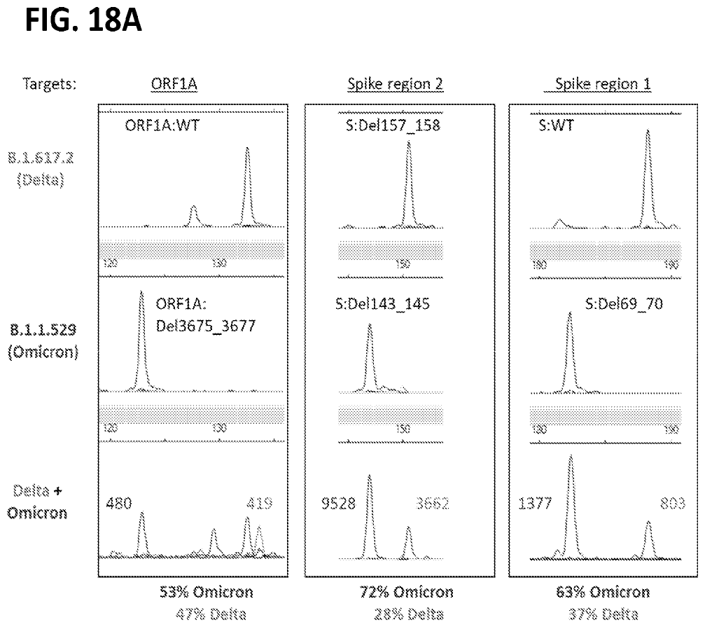

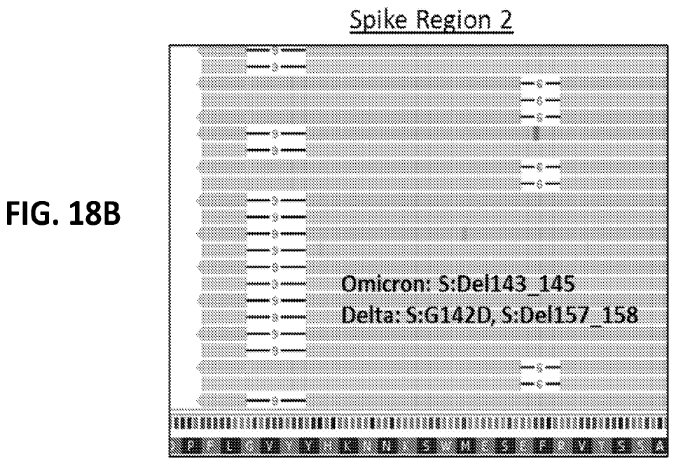

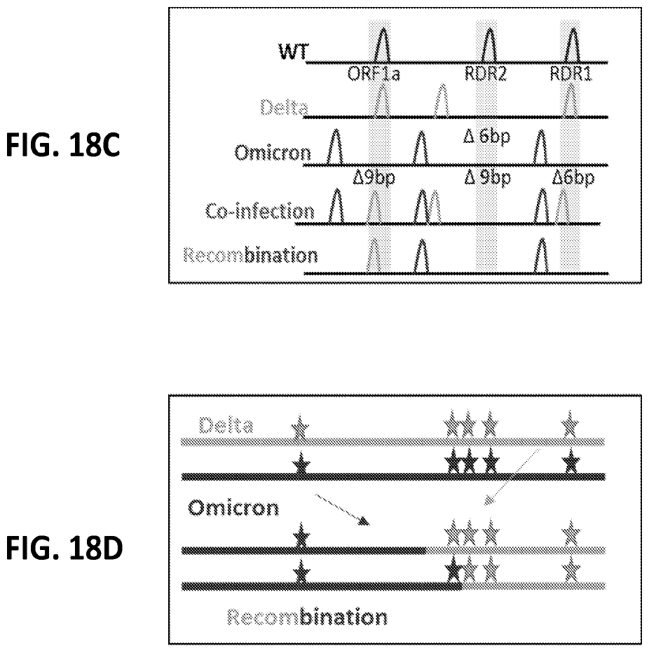

Forty-five days later (early January 2022), he returned with fever, worsening shortness of breath, and productive cough. The case patient again showed SARS-CoV-2 PCR positivity, but given the proximity to prior infection, was initially thought to be a result of persistent viral shedding. The patient was treated for hospital acquired pneumonia with piperacillin-tazobactam and vancomycin and methylprednisolone for acute cellular rejection. However, SARS-Cov-2 genotyping of the second PCR-positive specimen identified the presence of two SARS-CoV-2 variants: Delta (remaining from his initial infection) and Omicron (suggestive of a secondary infection) (FIG. 18A). As the Omicron variant had not yet emerged when the patient had initial SARS-CoV-2 infection, the genotyping and epidemiologic data indicated a novel infection on top of a persistent Delta infection. With confirmation of active SARS-CoV-2 disease, a course of Dexamethasone and Remdesivir was recommended to improve the symptomology in the case patient. The patient later given prophylactic monoclonal antibody therapy (tixagevimab/cilgavimab). With discontinued antibiotics and tapered steroids, the patient was discharged without supplemental oxygen and a FEV1 near baseline.

### Laboratory Findings and Genetic Relationship of the Patient's SARS-CoV-2 Variants

Variant status was determined for SARS-CoV-2 PCR-positive through a combination of WGS and a genotyping PCR analyzed by fragment analysis. Briefly, for fragment analysis, mutational hotspots of the SARS-CoV-2 genome were amplified with fluorescently labeled primers. These PCR amplicons (fragments) were separated by capillary electrophoresis, and size differences determine the presence or absences of characteristic deletions. The initial PCR-positive specimen solely contained Delta variant sequences, while the patient's subsequent specimen contained mutational signatures consistent with co-infection by the initial Delta VOC and the newly emerged Omicron VOC (B.1.1.529). Mutations specific to Delta and Omicron were found on independent reads using WGS (FIG. 18B), which indicates non-recombined viral infection. While a total of eight fragment analysis targets are used, only three are highlighted to demonstrate the difference in Omicron and Delta variants. ORF1A detects the 3 amino acid deletion from ORF1A:Del3675_3677. Spike recurrently deleted region (RDR)1 and 2 detect S:Del69_70 and mutations in the 140-160 amino acid range, respectively. The Delta variant has a 2 amino acid deletion in Spike RDR2 (S:Del157_158), while Omicron has a 3 amino acid deletion (S:Del143_145). No mutagenic signatures were identified by either genotyping or NGS to suggest recombination between the two lineages (FIG. 18C).

### Defective Nucleocapsid IgG Response Despite Infection

During both infections, the patient had undetectable IgG and IgM antibodies against nucleocapsid (FIG. 17A), which is useful for distinguishing infection-associated immune response from vaccine-related response. He was given monoclonal antibody therapy (Casirivimab and Imdevimab) during both infections, resulting in very high Spike IgG antibody levels. Notably, Casirivimab and Imdevimab are human monoclonal antibodies that bind to nonoverlapping epitopes of the S protein RBD of SARS-CoV-2 (anti-SARS-CoV-2 antibodies). The lack of nucleocapsid antibody response reflects an inability to mount an antibody response due to immunosuppression. Several studies have previously reported that patients with pre-existing immunosuppressive conditions or those who undergo immunosuppressive treatments exhibit low or no ability to mount antibody response.

### Additional Cases of SARS-CoV-2 Co-Infections

Following the identification of the primary case reported in this example, SARS-CoV-2 specimens were prospectively monitored for additional cases of SARS-CoV-2 co-infections with different VOCs. While the above case is reported in the greatest level of clinical detail, another case of Delta/Omicron coinfection was found in a 70-year-old female with uncontrolled diabetes (Diagnosed 1st week of January 2022).

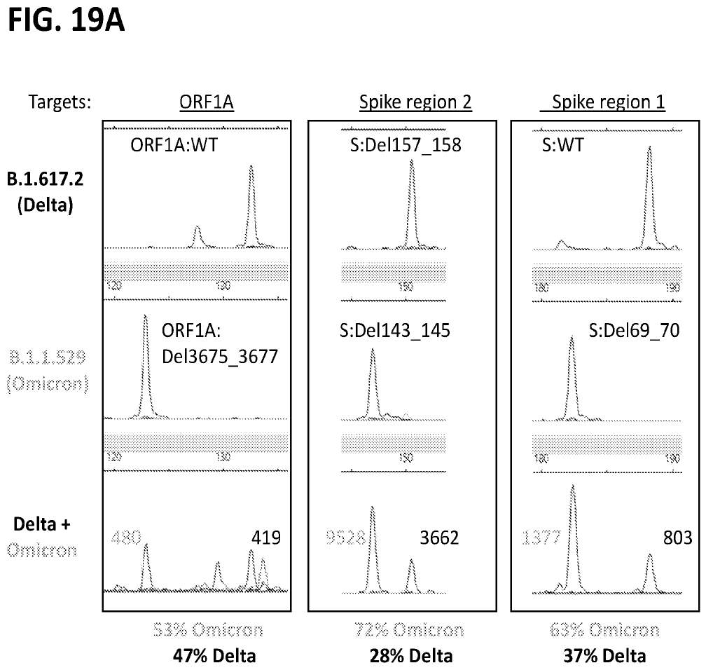

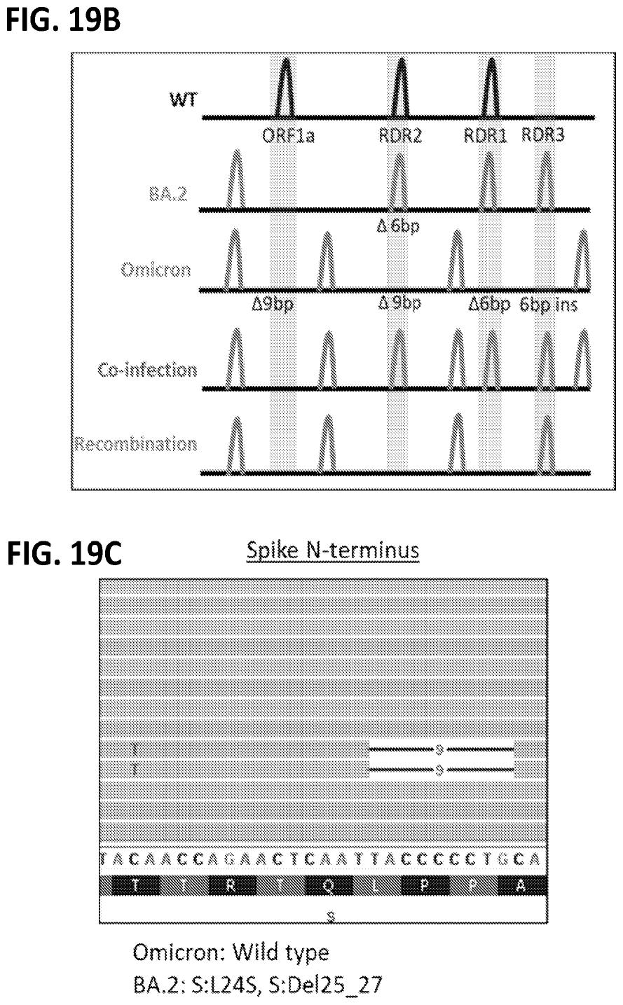

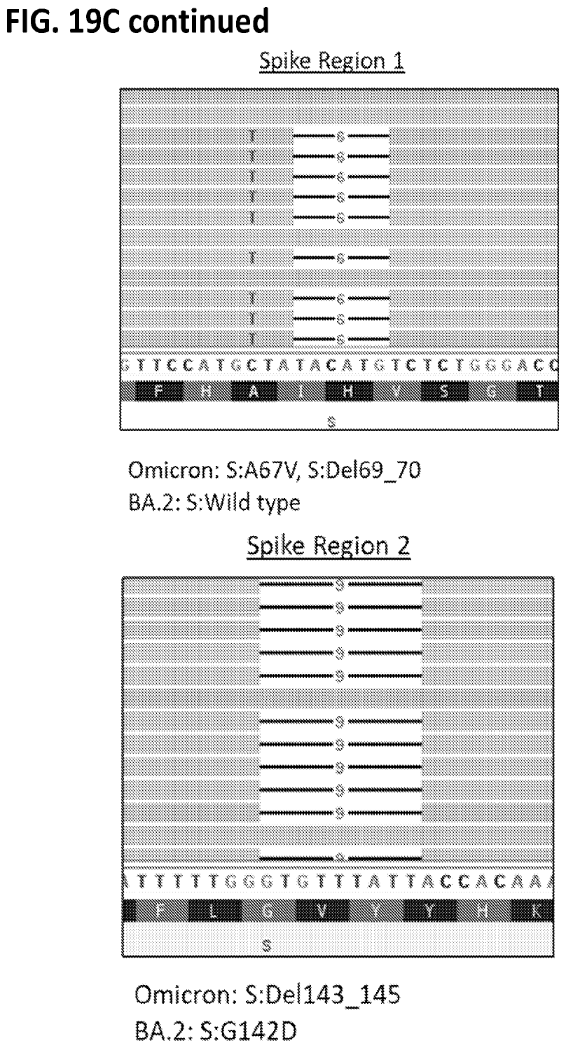

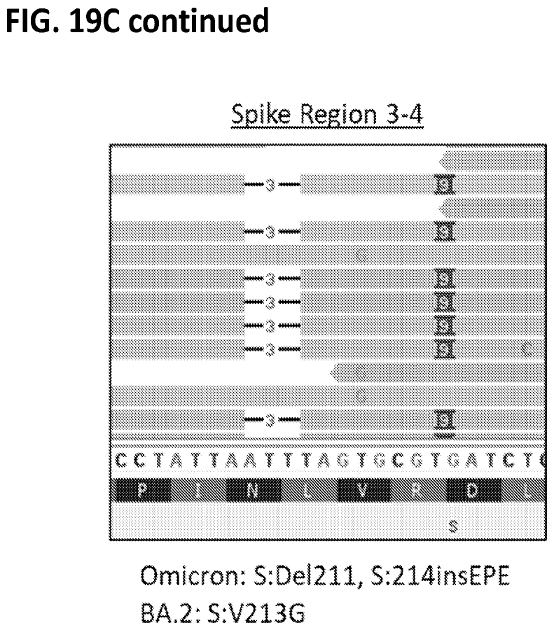

Samples from two independent patients revealed multiple peaks amplified for RDR1 (WT and 6b.p. deletion), RDR2 (WT and 9 b.p. deletion), RDR3-4 (WT and a 6 b.p. insertion) (FIG. 19A). This pattern could occur with Omicron and BA.2 variant genomic RNA present in the same reaction arising from either co-infection or contamination. Carry-over contamination has been a frequent issue in WGS methods but has not occurred in the fragment analysis PCR test (as determined during CLIA validation). To rule out the possibility of contamination, RNA was re-extracted from each sample and repeated. The confirmatory fragment analysis results were the same.

The co-occurrence of amplicons specific to the Omicron and BA.2 variants indicates a co-infection rather than recombination has occurred. Determining whether a co-infection is present by whole genome sequencing requires mutations be within 100-150 base pairs of each other to observe co-occurrence of mutations on the same read strand, we focused on the spike gene where multiple differences in mutations were found.

Sequencing showed separate forward and reverse reads (colored red and blue respectively, FIG. 19B) for the Omicron mutations (S:69_70Del and A67V) and BA.2 (no mutations present) in the RDR1 region. In the RDR2 region, the Omicron deletion (S:143_145Del) is present with alternative reads from the BA.2 variant (S:G142D). In the RDR3-4 region, separate reads are observed with mutations indicative of for Omicron (S:211 Del, 214 insEPE) and BA.2 (S:V213G). Lastly, in the N-terminal portion of the S gene, an S:L24S, S:25_27Del variants from BA.2 are present with sequence consistent with the Omicron variant (no mutation).

### Discussion

This example shows two cases of Delta/Omicron co-infection and two cases of Omicron/BA.2. While infectivity and replication dynamics of individual SARS-CoV-2 genetic lineages continue to be elucidated, significantly less is known concerning co-infections with unique SARS-CoV-2 variants. The paucity of information in the literature is likely multifactorial, given an epidemiological pattern of variant emergence and nonuniform application or access to sequencing or genotyping methods. Given the recent history of Delta-confirmed infection in November, the finding of Delta-Omicron co-infection appears authentic as a staggered type of co-infection and not the result of laboratory contamination. Testing of re-extracted sample helped rule-out laboratory contamination. In concordance with these findings, prior studies have also identified co-infection as an impetus for recombination between different SARS-CoV-2 variants (14,15).

As this is the first reported case report of Omicron/BA.2 variant co-infections so soon after an Omicron/Delta co-infection, it is predicted that such co-infections will continue to occur when one variant takes over another. Specifically, this is likely to occur in IC hosts who have trouble clearing the virus, thus, allowing it to effectively adapt by exploiting the host cell conditions. In this context, host proteins, sirtuin 1 and 5 (SIRT1 and SIRT5) involved in energy, metabolism, and survival processes, have been recently shown to interact with SARS-CoV-2 Nsp14 and generating a lethal phenotype of SARS-CoV-2 with effective replication and/or long-term propagation. Importantly, the expression and activity of SIRT1 was shown to be enhanced by the steroid, prednisolone, which the case patient has been receiving since transplant at a dose of 7.5 mg daily until COVID #1 and continued with 60 mg daily following COVID #1 (11/29/21 through 12/3/21). Thus, any such alterations in host cell pathways due to IC and/or IS drugs that can interfere with and dampen the SARS-CoV-2 nsp14-ExoN is likely to hamper the intrinsic fidelity and contribute to high-level mutagenesis.

The small number of cases observed prevents generalizable comments on whether co-infection protracts disease course or has more severe symptoms. The serological responses among IC patients are dampened as exemplified by the lack of Nucleocapsid IgG antibody response after months of primary and secondary infection in this lung transplant patient. The high anti-Spike IgG levels were due to monoclonal antibody use.

A key learning point of these cases is that protracted disease presents an opportunity for additional exposure or the emergence of new variants. Of note, it has been reported that persistently infected IC patients accumulate amino acid substitutions or deletions in different regions of human SARS-CoV-2 spike protein and thus conceived to be a source of new immune-escape viral variants (5,18,19). Also, CD4+ T-cell depletion (<20 CD4+ T-cell counts) as found in IC persons, including persons living with HIV/AIDS have been determined to be a risk of giving rise to SARS-CoV-2 resistance mutations (6-7). In contrast, an absent immune response does not reject virus and thus there is no competitive advantage for acquiring resistance mutations. Furthermore, these cases of SARS-CoV-2 co-infection may be more likely to occur in times when there is a diversity of variants in circulation. Thus, co-infections will continue to occur and methods to monitor this should be considered.

The most commonly used variant detection method is currently WGS. However, criteria for calling variants are that mutations must be detected at >50% or higher levels of variant allele frequencies. Additionally, due to biases introduced in amplicon-based library preparation, whole genome sequencing cannot accurately quantify differences in variant levels. This cut-off prevents minor allele-frequencies to be counted for detection of co-infections. Genotyping methods offer a sensitive alternative where multiple mutations can be detected at once provided a wild-type and mutant allele can be detected simultaneously. In this scenario, fragment analysis is superior, because it can detect multiple types of deletion mutations simultaneously. This method of multiplex SARS-CoV-2 genotyping by fragment analysis lends itself to not only detecting concurrent variant mutational signatures but also allows relative quantitation of the genomic material present. This quantitation can be cross applied to the CT value to infer whether an infection is active or not. For our patient, a 25% decrease in Delta nucleic acid corresponds with a 4-fold difference (2 CT value difference), which indicates the Delta variant was still active and not shedding dead virus.

Furthermore, genomic recombination can be screened by fragment analysis. The variant identification program, Pangolin, cannot distinguish recombination events, although it now recognizes some recently discovered recombinant variants named: XD, XE, and XF. However, this fragment analysis assay has targets across the genome from ORF1A to spike and ORF8/Nucleocapsid genes, where differences in a specific region may indicate recombination. Most assays just target the spike gene and recombination can occur across the entire SARS-CoV-2 genome.

Therefore, SARS-CoV-2 co-infections will continue and represent a source of viral evolution, which can be effectively monitored using a fragment analysis genotyping approach.

SEQUENCES

| | | SEQ ID |
|---|---|---|
| Name | Sequence | NO: |
| S69/70_F: | 5′-FAM-cgtggtgtttattaccctgacaaag-3′ | 1 |
| S69/70_R: | 5′-tcagtggaagcaaaataaacaccat-3′ | 2 |
| S144_F: | 5′-FAM-acgctactaatgttgttattaaagtct-3′ | 3 |
| S144_R: | 5′-tctgaactcactttccatccaact-3′ | 4 |
| ORF1A_F: | 5′-FAM-tgcctgctagttgggtgatg-3′ | 5 |
| ORF1A_R: | 5′-tgctgtcataaggattagtaacact-3′ | 6 |
| N1_F: | 5′-FAM-gaccccaaaatcagcgaaat-3′ | 7 |
| N1_R: | 5′-tctggttactgccagttgaatctg-3′ | 8 |
| Common_230_R | cagtagtgtcagcaatgtctctgcc | 9 |
| N501WT | 5′ FAM-caatcatatggtttccaacccacta | 10 |
| UTSW_N501Y_3CT | 5′ HEX-caatcatatggtttccaacccattt | 11 |
| UTSW_E484WT_2′TC | 5′ FAM-ggtagcacaccttgtaatggtgtcg | 12 |
| UTSW_E484K | 5′ HEX-cggtagcacaccttgtaatggtgtta | 13 |
| Yale_144del_F: | 5′-FAM-gaatttgaatgatccatttttgggtg | 14 |
| (RDR_F) | | |
| UTSW_RDR2_R: | 5′-ggctgagagacatattcaaagtgc | 15 |
| UTSW_ORF1A_F + 1: | 5′-FAM-tgcctgctagttgggtgatg | 16 |
| UTSW_ORF1A_R + 5: | 5′-gttcttgctgtcataaggattagtaacac | 17 |
| UTSW_L452WT_3′G | 5′-FAM-ctaaggttggtggtaattataattagct | 18 |
| UTSW_L452R | 5′-HEX-ctaaggttggtggtaattataattaccg | 19 |
| UTSW_RDR3-4_F | 5′-FAM-ggaccttgaaggaaaacaggg | 20 |
| UTSW_RDR3-4_R | 5′-tgaagataacccacataataagctgc | 21 |
| UTSW_ORF8_F | 5′-FAM-gccagaaacctaaattgggtagt | 22 |
| UTSW_ORF8_R | 5′-cccactgcgttctccattctg | 23 |
| N501_WT2TA | catatggtttccaacccacaa | 24 |
| N501y2TA | catatggtttccaacccacat | 25 |
| N501_Mut | caatcatatggtttccaacccactt | 26 |
| N501_WT2TAnew | aatcatatggtttccaacccacAa | 27 |
| N501_WT2TG | aatcatatggtttccaacccacGt | 28 |
| UTSW_N501Y_2TA | caatcatatggtttccaacccacAt | 29 |
| UTSW_N501Y_2TG | caatcatatggtttccaacccacGt | 30 |
| UTSW_N501Y_3CA | caatcatatggtttccaacccaAtt | 31 |
| UTSW_N501Y_3CG | caatcatatggtttccaacccaGtt | 32 |
| WT_2′TG | ggtagcacaccttgtaatggtgtGg | 33 |
| E484K_2′TG | ggtagcacaccttgtaatggtgtGa | 34 |
| E484K_2′TC | ggtagcacaccttgtaatggtgtCa | 35 |
| E484K_3′TA | ggtagcacaccttgtaatggtgAta | 36 |
| UTSW_E484K_3′TC | ggtagcacaccttgtaatggtgCta | 37 |
| E484K_3′TG | ggtagcacaccttgtaatggtgGta | 38 |
| FAM_UTSW_452_WT | ctaaggttggtggtaattataattaccT | 39 |
| 452_WT2′A | ctaaggttggtggtaattataattacAT | 40 |
| 452_WT2′T | ctaaggttggtggtaattataattacTT | 41 |
| 452_WT2′G | ctaaggttggtggtaattataattacGT | 42 |
| 452_WT3′A | ctaaggttggtggtaattataattaAcT | 43 |
| 452_WT3′T | ctaaggttggtggtaattataattaTcT | 44 |
| L452R_2′A | ctaaggttggtggtaattataattacAG | 45 |
| L452R_2′G | ctaaggttggtggtaattataattacGG | 46 |
| L452R_2′T | ctaaggttggtggtaattataattacTG | 47 |
| L452R_3′A | ctaaggttggtggtaattataattaAcG | 48 |
| HEX_UTSW_L452R_3′G | ctaaggttggtggtaattataattaGcG | 49 |
| L452R_3′T | ctaaggttggtggtaattataattaTcG | 50 |
| NCBI Reference | See Sequence Listing | 51 |
| Sequence NC_045512.2 | | |
| (Severe acute | | |
| respiratory syndrome | | |
| coronavirus 2 isolate | | |
| Wuhan-Hu-1, complete | | |
| genome | | |
| NCBI Reference | See Sequence Listing | 52 |
| Sequence: | | |
| YP_009724390.1 | | |
| (surface glycoprotein | | |
| [Severe acute respiratory | | |
| syndrome coronavirus 2) | | |

1. A composition for amplifying a SARS-COV-2 nucleic acid, the composition comprising at least one set of nucleic acid primers (primer set) selected from:
(i) a first primer set comprising a first nucleic acid at least 15 nucleotides in length and having at least 85% sequence identity to SEQ ID NO: 1, and a second nucleic acid at least 15 nucleotides in length and having at least 85% sequence identity to SEQ ID NO: 2,
(ii) a second primer set comprising a first nucleic acid at least 15 nucleotides in length and having at least 85% sequence identity to SEQ ID NO: 14, and a second nucleic acid at least 15 nucleotides in length and having at least 85% sequence identity to SEQ ID NO:15,
(iii) a third primer set comprising a first nucleic acid at least 15 nucleotides in length and having at least 85% sequence identity to SEQ ID NO: 20, and a second nucleic acid at least 15 nucleotides in length and having at least 85% sequence identity to SEQ ID NO: 21,
(iv) a fourth primer set comprising a first nucleic acid at least 15 nucleotides in length and having at least 85% sequence identity to SEQ ID NO: 16, and a second nucleic acid at least 15 nucleotides in length and having at least 85% sequence identity to SEQ ID NO: 17,
(v) a fifth primer set comprising a first nucleic acid at least 15 nucleotides in length and having at least 85% sequence identity to SEQ ID NO: 22, and a second nucleic acid at least 15 nucleotides in length and having at least 85% sequence identity to SEQ ID NO: 23,
(vi) a sixth primer set comprising a first nucleic acid at least 15 nucleotides in length and having at least 85% sequence identity to SEQ ID NO: 9, a second nucleic acid at least 15 nucleotides in length and having 85% sequence identity to SEQ ID NO: 10, and a third nucleic acid consisting of up to 15 nucleotides and having at least 85% sequence identity to SEQ ID NO: 11,
(vii) a seventh primer set comprising a first nucleic acid at least 15 nucleotides in length and having at least 85% sequence identity to SEQ ID NO: 9, a second nucleic acid at least 15 nucleotides in length and having 85% sequence identity to SEQ ID NO: 12, and a third nucleic acid at least 15 nucleotides in length and having 85% sequence identity to SEQ ID NO: 13, and
(viii) an eighth primer set comprising a first nucleic acid at least 15 nucleotides in length and having at least 85% sequence identity to SEQ ID NO: 9, a second nucleic acid at least 15 nucleotides in length and having at least 85% sequence identity to SEQ ID NO: 18, and a third nucleic acid at least 15 nucleotides in length and having 85% sequence identity to SEQ ID NO: 19.

2-6. (canceled)

7. A method for detecting a SARS-COV-2 variant, the method comprising: (a) amplifying a nucleic acid in a sample suspected of comprising the SARS-COV-2 variant, the variant comprising at least one insertion, deletion or single nucleotide polymorphism (SNP) as compared to a wildtype SARS-COV-2 nucleic acid, and (b) detecting an amplicon produced in (a); wherein amplifying the nucleic acid comprises:
(i) performing a polymerase chain reaction using at least one set of nucleic acids (primer set), wherein the primer set amplifies a nucleic acid of the SARS-COV-2 variant comprising the insertion or deletion and the amplicon detected in (b) is larger or smaller than a corresponding amplicon generated from a wildtype SARS-COV-2 nucleic acid; and/or
(ii) performing a polymerase chain reaction using a first nucleic acid primer, a second nucleic acid primer and third nucleic acid primer, wherein the first nucleic acid primer preferentially anneals to a nucleic acid comprising a mutant allele of the SNP, the second nucleic acid primer preferentially anneals to a nucleic acid comprising wildtype allele of the SNP, and the third nucleic acid allows for the amplification of a nucleic acid comprising the SNP.

8. The method of claim 7, wherein the insertion or deletion is located in a recurrently deleted region 1 (RDR1), a recurrently deleted region 2 (RDR2), a recurrently deleted region 34 (RDR3_4), an open reading frame 1A (ORF1A), or an open reading frame 8 (ORF8) and/or wherein the single nucleotide polymorphism encodes an amino acid substitution in a protein of the SARS-COV-2 virus, the amino acid substitution selected from N501Y, L452R, or E484K according to reference SEQ ID NO: 52.

9. The method of claim 8, wherein:
(a) the insertion or deletion is located in the RDR1 and the primer set in (i) comprises a first nucleic acid at least 15 nucleotides in length and comprising at least 85% sequence homology with SEQ ID NO: 1; and a second nucleic acid at least 15 nucleotides in length and comprising at least 85% sequence homology with SEQ ID NO: 2; or
(b) the insertion or deletion is located in the RDR2, and the primer set in (i) comprises a first nucleic acid at least 15 nucleotides in length and comprising at least 85% sequence homology with SEQ ID NO: 14; and a second nucleic acid at least 15 nucleotides in length and comprising at least 85% sequence homology with SEQ ID NO: 15: or
(c) the insertion or deletion is located in the RDR3 4 and the primer set in (i) comprises a first nucleic acid at least 15 nucleotides in length and comprising at least 85% sequence homology with SEQ ID NO: 20; and a second nucleic acid at least 15 nucleotides in length and comprising at least 85% sequence homology with SEQ ID NO: 21: or
(d) the insertion or deletion is located in the ORF1A and the primer set in (i) comprises a first nucleic acid at least 15 nucleotides in length and comprising at least 85% sequence homology with SEQ ID NO: 16; and a second nucleic acid at least 15 nucleotides in length and comprising at least 85% sequence homology with SEQ ID NO: 17; or
(e) wherein the insertion or deletion is located in the ORF8 and the primer set in (i) comprises a first nucleic acid at least 15 nucleotides in length and comprising at least 85% sequence homology with SEQ ID NO: 22; and a second nucleic acid at least 15 nucleotides in length and comprising at least 85% sequence homology with SEQ ID NO: 23.

10. The method of claim 9, wherein:
(a) the insertion or deletion in the RDR1 comprises a 6 base pair deletion or a 6 base pair insertion; or
(b) the insertion or deletion in RDR2 comprises a 3 base pair deletion, a 9 base pair deletion, or a 3 base pair insertion; or
(c) the insertion or deletion in RDR 3_4 comprises a 9 base pair deletion, a 6 base pair insertion or a 21 base pair deletion; or
(d) the insertion or deletion in ORF1A comprises a 9 base pair deletion; or
(e) the insertion or deletion in ORF8 comprises a 4 base pair insertion or a 6 base pair deletion.

11.-18. (canceled)

19. The method of claim 9, wherein the amplicon produced in (b) is larger or smaller as compared to a corresponding amplicon produced by amplifying a wildtype SARS-COV-2 nucleic acid in the presence of the at least one primer pair and wherein the method further comprises identifying the SARS-CO-V-2 variant by detecting the larger or smaller amplicon as compared to a control sample comprising a wildtype SARS-COV-2 nucleic acid.

20. The method of claim 7, wherein:
(a) the single nucleotide polymorphism results in a N501Y amino acid substitution in the encoded protein, and the primer set in (ii) comprises a first nucleic acid at least 15 nucleotides in length and comprising at least 85% sequence homology with SEQ ID NO: 9; and a second nucleic acid at least 15 nucleotides in length and comprising at least 85% sequence homology with SEQ ID NO: 10, and a third nucleic acid at least 15 nucleotides in length and comprising at least 85% sequence homology with SEQ ID NO 11; and/or
(b) the single nucleotide polymorphism results in a E484K amino acid substitution in the encoded protein, and the primer set in (ii) comprises a first nucleic acid at least 15 nucleotides in length and having at least 85% sequence identity to SEQ ID NO: 9, a second nucleic acid at least 15 nucleotides in length and having 85% sequence identity to SEQ ID NO: 12, and a third nucleic acid at least 15 nucleotides in length and having 85% sequence identity to SEQ ID NO: 13; and/or
(c) wherein the single nucleotide polymorphism results in a L452R amino acid substitution in the encoded protein, and the primer set in (ii) comprises a first nucleic acid at least 15 nucleotides in length and having at least 85% sequence identity to SEQ ID NO: 9, a second nucleic acid at least 15 nucleotides in length and having at least 85% sequence identity to SEQ ID NO: 18, and a third nucleic acid at least 15 nucleotides in length and having 85% sequence identity to SEQ ID NO: 19.

21-22. (canceled)

23. The method of claim 7, wherein the first nucleic acid in (ii) is labeled with a first fluorescent marker and the second nucleic acid in (ii) is labeled with a second fluorescent marker, wherein the first and second fluorescent markers are independently detectable in a single sample and wherein the method further comprises identifying the SARS-COV-2 variant when a ratio of the first fluorescent marker to the second fluorescent marker exceeds a threshold.

24. The method of claim 7, comprising amplifying one or more the nucleic acids in a sample suspected of comprising the SARS-COV-2 variant using at least one set, at least two sets, at least three sets, at least four sets, at least five sets, at least six sets, at least seven sets, or at least eight sets of nucleic acid primers (primer sets) selected from:
(i) a first primer set to detect an insertion or deletion in RDR1 comprising a first nucleic acid at least 15 nucleotides in length and having at least 85% sequence identity to SEQ ID NO: 1, and a second nucleic acid at least 15 nucleotides in length and having at least 85% sequence identity to SEQ ID NO: 2,
(ii) a second primer set to detect an insertion or deletion in RDR2 comprising a first nucleic acid at least 15 nucleotides in length and having at least 85% sequence identity to SEQ ID NO: 14, and a second nucleic acid at least 15 nucleotides in length and having at least 85% sequence identity to SEQ ID NO:15,
(iii) a third primer set to detect an insertion or deletion in RDR3_4 comprising a first nucleic acid at least 15 nucleotides in length and having at least 85% sequence identity to SEQ ID NO: 20, and a second nucleic acid at least 15 nucleotides in length and having at least 85% sequence identity to SEQ ID NO: 21,
(iv) a fourth primer set to detect an insertion or deletion in ORF1 a comprising a first nucleic acid at least 15 nucleotides in length and having at least 85% sequence identity to SEQ ID NO: 16, and a second nucleic acid at least 15 nucleotides in length and having at least 85% sequence identity to SEQ ID NO: 17,
(v) a fifth primer set to detect an insertion or deletion in ORF8 comprising a first nucleic acid at least 15 nucleotides in length and having at least 85% sequence identity to SEQ ID NO: 22, and a second nucleic acid at least 15 nucleotides in length and having at least 85% sequence identity to SEQ ID NO: 23,
(vi) a sixth primer set to detect an N501Y SNP comprising a first nucleic acid at least 15 nucleotides in length and having at least 85% sequence identity to SEQ ID NO: 9, a second nucleic acid at least 15 nucleotides in length and having 85% sequence identity to SEQ ID NO: 10, and a third nucleic acid at least 15 nucleotides in length and having at least 85% sequence identity to SEQ ID NO: 11,
(vii) a seventh primer set to detect an E484K SNP comprising a first nucleic acid at least 15 nucleotides in length and having at least 85% sequence identity to SEQ ID NO: 9, a second nucleic acid at least 15 nucleotides in length and having 85% sequence identity to SEQ ID NO: 12, and a third nucleic acid at least 15 nucleotides in length and having 85% sequence identity to SEQ ID NO: 13 and
(viii) an eighth primer set to detect a L452R SNP comprising a first nucleic acid at least 15 nucleotides in length and having at least 85% sequence identity to SEQ ID NO: 9, a second nucleic acid at least 15 nucleotides in length and having at least 85% sequence identity to SEQ ID NO: 18, and a third nucleic acid at least 15 nucleotides in length and having 85% sequence identity to SEQ ID NO: 19; and wherein the SARS-COV-2 variant is identified based on the presence or absence of the insertion or deletion in RDR1, RDR2, RDR3_4, ORF1a, ORF8, or the presence or absence of the N501Y, NE484K, or L452R SNPs.

25. The method of claim 24, wherein the SARS-COV-2 variant is identified as an alpha variant, a beta variant, a gamma variant, an iota variant, a lambda variant, a delta variant, or an omicron variant.

26. The method of claim 25, wherein the SARS-COV-2 variant is identified as:
(a) an alpha variant when a 6 base pair deletion is detected in RDR1, a 3 base pair deletion is detected in RDR2, a 9 base pair deletion is detected in ORF1A, and an N501Y single nucleotide polymorphism is detected; or
(b) a beta variant when a 9 base pair deletion is detected in RDR3 4, a 9 base pair deletion is detected in ORF1A, and N501Y and E484K single nucleotide polymorphisms are detected; or
(c) a gamma variant when a 4 base pair insertion is detected in ORF8, a 9 base pair deletion is detected in ORF1A, and N501Y and E484K single nucleotide polymorphisms are detected; or
(d) an iota variant when a 9 base pair deletion is detected in ORF1A, and an E484K single nucleotide polymorphism is detected; or
(e) a lambda variant when a 9 base pair deletion is detected in ORF1A, a 21 base pair deletion is detected in RDR3 4 and reduced amplification of a mutant allele at position L452 is observed; or
(f) an omicron variant when a 6 base pair deletion is detected in RDR1, a 9 base pair deletion is detected in RDR2, a 6 base pair insertion is detected in RDR3 4, a 9 base pair deletion is detected in ORF1A, and an N501Y single nucleotide polymorphism is detected; or
(q) a mu variant when a 3 base pair insertion is detected in RDR2, and an N501Y and E484K single nucleotide Polymorphisms are detected: or
(h) a delta variant when a 6 base pair deletion is detected in RDR2⁻, a 6 base pair deletion is detected in ORF8, a L452R single nucleotide Polymorphism is detected, and amplification of a wildtype allele at position E484 is reduced without an increase in amplification of a mutant allele.

27-33. (canceled)

34. The method of claim 7, wherein detecting the amplicon in (b) comprises capillary electrophoresis.

35. The method of claim 7, wherein amplifying the nucleic acid in (a) and/or detecting the amplicon in (b) comprises using real-time PCR (RT-PCR).

36. The method of claim 7, comprising detecting more than one SARS-COV-2 variant in a single sample.

37. The method of claim 7, wherein the sample is selected from a nasopharyngeal swab sample, an oropharyngeal sample, a nasal aspirate sample, a nasal or mid-turbinate swab, a nasal aspirate sample, a nasal wash sample, a throat swab sample, a bronchoalveolar lavage sample, a bronchial aspirate sample, a bronchial wash sample, an endotracheal aspirate, an endotracheal wash sample, a tracheal aspirate, a nasal secretion sample, a mucus sample, a sputum sample, a lung tissue samples, a urine sample, a saliva sample, and a fecal sample.

38. A method of generating a primer for detecting a single nucleotide polymorphism (SNP) in a nucleic acid, the method comprising:
(a) providing a first nucleic acid sequence complementary to a nucleic acid comprising a mutant allele of the SNP, wherein the first nucleic acid sequence comprises a terminal 3′ nucleotide complementary to either the mutant or the wildtype nucleotide at the location of the SNP;
(b) substituting an adenine (A) for a thymine (T) (T>A substitution) one base pair upstream (5′ direction) of the terminal 3′ nucleotide complementary to the mutant or wildtype nucleotide at the location of the SNP to generate a mutant primer sequence or a wildtype primer sequence; and
(c) generating a mutant primer comprising the mutant primer sequence and/or a wildtype primer comprising the wildtype primer sequence;
wherein binding of the mutant primer to a wildtype allele or the binding of the wildtype primer to a mutant allele is destabilized as compared to a mutant primer or wildtype primer without the T>A substitution.

39. A method of generating a primer for detecting a single nucleotide polymorphism (SNP) in a nucleic acid, the method comprising:
(a) providing a first nucleic acid sequence complementary to a nucleic acid comprising a mutant allele of the SNP, wherein the first nucleic acid sequence comprises a terminal 3′ nucleotide complementary to the mutant nucleotide at the location of the SNP;
(b) generating a mutant primer sequence by substituting an adenine (A) for a cytosine (C) (C>A substitution) two base pairs upstream (5′ direction) of the terminal 3′ nucleotide that is complementary to the mutant allele; and
(c) preparing an isolated nucleic acid primer (mutant primer) comprising the mutant primer sequence, wherein the mutant primer binds the mutant allele with a higher specificity than an equivalent primer without the C>A substitution.

40. An isolated nucleic acid primer comprising a nucleotide sequence consisting of any one of SEQ ID NOs: 1 to 50.

41. The isolated nucleic acid primer of claim 40, wherein the nucleotide sequence consists of:
b) any one of SEQ ID NOs: 1, 2, 14, 15, 16, 17, 9, 10, 11, 12, 13, 18, 19, 20, 21, 22, and 23; or
(c) any one of SEQ ID NOs: 10, 11, 24, 25, 26, 27, 28, 29, 30, 31, and 32; or
(d) any one of SEQ ID NOs: 12, 13, 33, 34, 35, 36, 37, and 38; or
(e) any one of SEQ ID NOs: 18, 19, 39, 40, 41, 42, 43, 44, 45, 46, 47, 48, 49, and 50.

42-46. (canceled)


SEQUENCE LISTING (52 sequences)

SEQ ID NO: 1 - DNA, 25 bp, Artificial Sequence
  Synthetic construct - primer (S69/70_F)
cgtggtgtttattaccctgacaaag

SEQ ID NO: 2 - DNA, 25 bp, Artificial Sequence
  Synthetic construct - primer (S69/70_R)
tcagtggaagcaaaataaacaccat

SEQ ID NO: 3 - DNA, 27 bp, Artificial Sequence
  Synthetic construct - primer (S144_F)
acgctactaatgttgttattaaagtct

SEQ ID NO: 4 - DNA, 24 bp, Artificial Sequence
  Synthetic construct - primer (S144_R)
tctgaactcactttccatccaact

SEQ ID NO: 5 - DNA, 20 bp, Artificial Sequence
  Synthetic construct - primer (ORF1A_F)
tgcctgctagttgggtgatg

SEQ ID NO: 6 - DNA, 25 bp, Artificial Sequence
  Synthetic construct - primer (ORF1A_R)
tgctgtcataaggattagtaacact

SEQ ID NO: 7 - DNA, 20 bp, Artificial Sequence
  Synthetic construct - primer (N1_F)
gaccccaaaatcagcgaaat

SEQ ID NO: 8 - DNA, 24 bp, Artificial Sequence
  Synthetic construct - primer (N1_R)
tctggttactgccagttgaatctg

SEQ ID NO: 9 - DNA, 25 bp, Artificial Sequence
  Synthetic construct - primer (Common_230_R)
cagtagtgtcagcaatgtctctgcc

SEQ ID NO: 10 - DNA, 25 bp, Artificial Sequence
  Synthetic construct - primer (N501WT)
caatcatatggtttccaacccacta

SEQ ID NO: 11 - DNA, 25 bp, Artificial Sequence
  Synthetic construct - primer (UTSW_N501Y_3CT)
caatcatatggtttccaacccattt

SEQ ID NO: 12 - DNA, 25 bp, Artificial Sequence
  Synthetic construct - primer (UTSW_E484WT_2′TC]
ggtagcacaccttgtaatggtgtcg

SEQ ID NO: 13 - DNA, 26 bp, Artificial Sequence
  Synthetic construct - primer (UTSW_E484K)
cggtagcacaccttgtaatggtgtta

SEQ ID NO: 14 - DNA, 26 bp, Artificial Sequence
  Synthetic construct - primer (Yale_144del_F (RDR_F))
gaatttgaatgatccatttttgggtg

SEQ ID NO: 15 - DNA, 24 bp, Artificial Sequence
  Synthetic construct - primer (UTSW_RDR2_R)
ggctgagagacatattcaaagtgc

SEQ ID NO: 16 - DNA, 20 bp, Artificial Sequence
  Synthetic construct - primer (UTSW_ORF1A_F+1)
tgcctgctagttgggtgatg

SEQ ID NO: 17 - DNA, 29 bp, Artificial Sequence
  Synthetic construct - primer (UTSW_ORF1A_R+5)
gttcttgctgtcataaggattagtaacac

SEQ ID NO: 18 - DNA, 28 bp, Artificial Sequence
  Synthetic construct - primer (UTSW_L452WT_3′G)
ctaaggttggtggtaattataattagct

SEQ ID NO: 19 - DNA, 28 bp, Artificial Sequence
  Synthetic construct - primer (UTSW_L452R)
ctaaggttggtggtaattataattaccg

SEQ ID NO: 20 - DNA, 21 bp, Artificial Sequence
  Synthetic construct - primer (UTSW_RDR3-4_F)
ggaccttgaaggaaaacaggg

SEQ ID NO: 21 - DNA, 26 bp, Artificial Sequence
  Synthetic construct - primer (UTSW_RDR3-4_R)
tgaagataacccacataataagctgc

SEQ ID NO: 22 - DNA, 23 bp, Artificial Sequence
  Synthetic construct - primer (UTSW_ORF8_F)
gccagaaacctaaattgggtagt

SEQ ID NO: 23 - DNA, 21 bp, Artificial Sequence
  Synthetic construct - primer (UTSW_ORF8_R)
cccactgcgttctccattctg

SEQ ID NO: 24 - DNA, 21 bp, Artificial Sequence
  Synthetic construct - primer (N501_WT2TA)
catatggtttccaacccacaa

SEQ ID NO: 25 - DNA, 21 bp, Artificial Sequence
  Synthetic construct - primer (N501y2TA)
catatggtttccaacccacat

SEQ ID NO: 26 - DNA, 25 bp, Artificial Sequence
  Synthetic construct - primer (N501_Mut)
caatcatatggtttccaacccactt

SEQ ID NO: 27 - DNA, 24 bp, Artificial Sequence
  Synthetic construct - primer (N501_WT2TAnew)
aatcatatggtttccaacccacaa

SEQ ID NO: 28 - DNA, 24 bp, Artificial Sequence
  Synthetic construct - primer (N501_WT2TG)
aatcatatggtttccaacccacgt

SEQ ID NO: 29 - DNA, 25 bp, Artificial Sequence
  Synthetic construct - primer (UTSW_N501Y_2TA)
caatcatatggtttccaacccacat

SEQ ID NO: 30 - DNA, 25 bp, Artificial Sequence
  Synthetic construct - primer (UTSW_N501Y_2TG)
caatcatatggtttccaacccacgt

SEQ ID NO: 31 - DNA, 25 bp, Artificial Sequence
  Synthetic construct - primer (UTSW_N501Y_3CA)
caatcatatggtttccaacccaatt

SEQ ID NO: 32 - DNA, 25 bp, Artificial Sequence
  Synthetic construct - primer (UTSW_N501Y_3CG)
caatcatatggtttccaacccagtt

SEQ ID NO: 33 - DNA, 25 bp, Artificial Sequence
  Synthetic construct - primer (WT_2′TG)
ggtagcacaccttgtaatggtgtgg

SEQ ID NO: 34 - DNA, 25 bp, Artificial Sequence
  Synthetic construct - primer (E484K_2′TG)
ggtagcacaccttgtaatggtgtga

SEQ ID NO: 35 - DNA, 25 bp, Artificial Sequence
  Synthetic construct - primer (E484K_2′TC)
ggtagcacaccttgtaatggtgtca

SEQ ID NO: 36 - DNA, 25 bp, Artificial Sequence
  Synthetic construct - primer (E484K_3′TA)
ggtagcacaccttgtaatggtgata

SEQ ID NO: 37 - DNA, 25 bp, Artificial Sequence
  Synthetic construct - primer (UTSW_E484K_3′TC)
ggtagcacaccttgtaatggtgcta

SEQ ID NO: 38 - DNA, 25 bp, Artificial Sequence
  Synthetic construct - primer (E484K_3′TG)
ggtagcacaccttgtaatggtggta

SEQ ID NO: 39 - DNA, 28 bp, Artificial Sequence
  Synthetic construct - primer (FAM_UTSW_452_WT)
ctaaggttggtggtaattataattacct

SEQ ID NO: 40 - DNA, 28 bp, Artificial Sequence
  Synthetic construct - primer (452_WT2′A)
ctaaggttggtggtaattataattacat

SEQ ID NO: 41 - DNA, 28 bp, Artificial Sequence
  Synthetic construct - primer (452_WT2′T)
ctaaggttggtggtaattataattactt

SEQ ID NO: 42 - DNA, 28 bp, Artificial Sequence
  Synthetic construct - primer (452_WT2′G)
ctaaggttggtggtaattataattacgt

SEQ ID NO: 43 - DNA, 28 bp, Artificial Sequence
  Synthetic construct - primer (452_WT3′A)
ctaaggttggtggtaattataattaact

SEQ ID NO: 44 - DNA, 28 bp, Artificial Sequence
  Synthetic construct - primer (452_WT3′T)
ctaaggttggtggtaattataattatct

SEQ ID NO: 45 - DNA, 28 bp, Artificial Sequence
  Synthetic construct - primer (L452R_2′A)
ctaaggttggtggtaattataattacag

SEQ ID NO: 46 - DNA, 28 bp, Artificial Sequence
  Synthetic construct - primer (L452R_2′G)
ctaaggttggtggtaattataattacgg

SEQ ID NO: 47 - DNA, 28 bp, Artificial Sequence
  Synthetic construct - primer (L452R_2′T)
ctaaggttggtggtaattataattactg

SEQ ID NO: 48 - DNA, 28 bp, Artificial Sequence
  Synthetic construct - primer (L452R_3′A)
ctaaggttggtggtaattataattaacg

SEQ ID NO: 49 - DNA, 28 bp, Artificial Sequence
  Synthetic construct - primer (HEX_UTSW_L452R_3′G)
ctaaggttggtggtaattataattagcg

SEQ ID NO: 50 - DNA, 28 bp, Artificial Sequence
  Synthetic construct - primer (L452R_3′T)
ctaaggttggtggtaattataattatcg

SEQ ID NO: 51 - DNA, 29903 bp, Severe acute respiratory syndrome coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 52 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT